SEQUENCE LISTING (9 sequences)

SEQ ID NO: 1 - DNA, 44096 bp, human
  mRNA (9)..(401): Exon 1
gcgtgtttgcgtacagcgcgagtggccgcgcgggcagagtcggagctcggagctgctgcttctggttctcttgtggccgccgtcgctgtccggctgccttgggctgccgaacagacaaggcgtgggccacagcacctcagaagccgacgcagctcgacgcaggggccggcaggagggtgggcgatcgcgtgtcggagggcgccgcgcgggcaggcgggcgggcgccagagggggaaagaggcgggggcggcgggtcagccgctggccgggccggccggggaatgtcgatgcctgacgcgatgccgctgcccggggtcggggaggagctgaagcaggccaaggagatcgaggacgccgagaagtactccttcatggccaccgtcaccaaggcgcccaagaaggtgcgggggctctgggtggcggcgggggctcgggctccggcctcgcggaagggacgcgagcaagcccgggctctggctagtttgggacttctgggggtgactggggacggcggaggtgatgtagggggtggcggctgtgcggccccaggcctctcccgggctgaaaggccgcctctgacctcgctttcctttgtccctcggcccaagtcctccgggatcgttttgaaacccgacagggccaggccttaaatttgcgactgacttttcctggatgaagggcctcgaagacgagagtgggagggggtgaaccaactcagctgacccttgtgaccagtccgggggcatggggtgtacagaaaatctcccttcaggtgagtgggtaaatctggcagaacttccatcaaatctttgggagcagtggccgctttatccaacaactgcgtgatagctgggaggcaagagaaaagaatgcgtgaagctagctctgatatacaggtgaaagcgatgagggtaacatgaaactgaaaatctatgattttcactttctacagcattccaattacttcgaaatatgtaagaaattgacatttggaggttggtgacagcagacactgtcatggatacaggtggatcctagagtcatgtgaatagatcacttaggcatccctggagaaaggagcttgggtgggttggagtagtgggtggtaagggaagattgctcactcttgactgtacttttgaggcagaacaagtttgatctattgtgctgtaggttgactcaaaggacacccttcgaattctctcataaatggcaacctttggagattttagtctgactagctctccgagtggtggaattgggatccttattgctttctcatcatttactgccaaccacatcttgattttccatacacctggcacccagttagcagtagaccagagaacctttacaaagaggtgaccttcctgaagccagtgagaacccagataaatgtctcccttctttataaagacagagtcttacaaagctgtctctgcctgcatccagtgtttaatgggatgcagggctgaacaaagcaagggctgcaggaatagttaagtccttgaggcttcaactagttaagctgtcttaagtgggagagatggctcttttttttttttttaagtgaattaacctgccactccagaaatcaaatgcaagatcaaaaaagacgagtcgccattgtgtgctttcgctgttttctcacaaaattaagaatttaattcctttaaaagattgtggaagattccttgactctgaggcatctgattaactttagtgctgagagttcagttgactgctttgttgggacaatgaccctttgcaatctgcactattctttggagaaacagtcacagtccttataagcagaatgattgagggtgggaccttagatgagttagaggaaagaatgggaaagccctaatacctgaatttcccagactctgtcttctttgctgttacttgcctttatgttagtaaatctgtattcttgaaagtaccactgttgataagacaaagtctatatcgtagattgtaaaaactcattaaggcagggattcttgttcacctttgtatccagtgtgcctagctttgtacccagcatacagtaggtactcaaatattaattgctcacctggctaactagtaactgagatgtccatgtggccacctaccatgaataactatcccttttcctctcttcttcccaggaggaatattactaatgtgtattattgtcaggccaagtgctgatgattttagacttatattgcattagctaaaagaagcaggctctgccaggaatgtgggaagaccctgttttaagtttccatttaccagtaaatgtgccccaccgtgtttcttattaatctttctcttctaagttttattctttagcatttacgtagtactttgtaataaagtgtttcatgatgactaattaccctttccctagtgatgataaaagacatttattaagttaatggatcagttgaggactagtcaaggtcacatagcaagtcctaggcaatctgtttttcagaactgctgaattgaacattaagacttaagtcacttttgtacctctgccatgtgttgataacgtgaaagcatgggaaaagtaagagggaaaaaagtaacttggcatggtacttaatggtgtgagaaaaagacctcttcaaatggtgcagtccttcacctatgagaattctaccctggctctagaccagtaatccttggattattctgaagccctgaggcccctcaggaattgaggatgttcttgatgccagaggatataggttacttcagggtgaccttgatggtcctcagttagatcagatcccagtgtcactttctggtcagcttttattcattttgtttgctttgtttgagacagagtcagcttttattcattttgtttgctttgtttgagatagagtggatgtccatcaattaaaagcataaatatctagattcttcagctataatcatgacttactcagttttgtatgccaaggtctatcacactgctatgaacaaaataggacttgttggtttgattgaagatttgagatgtctgggtgtcgagaagtcaactaaagggatatgaccttgagcaaattaattagcttttgtacctgcatctcctcatctgccaaataagtgcctattttttttggaacaggtggagataggggcatgttggggagttaagatagcagtgtaattttttaaaagtctattcgtatatttatattctttcttcaatgggcatttgaagtggcttataacacagaacatatatgtgatataaaaggaagtaaaaactatagaaagaatgaaaaaaatattgttaacctcgagggctaagatagttgatgaggttgatcacatgcaacttgaaacttttagcagccagaggatgaaaagaatcatagttacagtctccttagcaggcatgggaaataatacatgtcaattcctcaaggggacgacttttcctgacacagagttctaaaataatttttcacgggaatgtcttaatagctgtagtgtaattcacattgtaaggcttgttcacgactgtcactggtgatttcagatggtatggctaggaaaatttgaggtaagaaaaggaatccttgagttttaagagttaagtgtaccacaactgaagaagatccttgtttaatgttggcagttatcggtcaaaccaagtaagtaatctagttccccataaccactggttatttatccctcttttgctgctactttgtgtaacaggagtggtaactgtccttggacctttggaaattgacactgtgtcaagttgattcttttttcatagtgtaaacccaaatctccaacttgttggagagctcttacaataagcatgtttctccggaaggcctttcagaacatttgctgttctctgaacttctgaacattaaaggcactctccagagtgatccattaaacattttgacctaggatctgtggagtcagacccatcagcaaaatgtggggaaggagaagagcacagaagaaactacttttgtttataggatattctttatttgaatgaagctatgtgccactaattcatcaaaagggtcagctgagacttaggaagcacaccaagtagagccccaatgggtacagcttgggtgttaacccgataggtaaaatccaggtttctccttttgtcatcagagttatcttacggttaatttttggtgcagttgtatagaatttacgccaggacttgtcagaatcactgaaaggagatttcataggttggcatttcctatcttcacatcattagtatctcttgattctccttagagtggctgctgttgtagtagcagtctttaaaacaggagttggggtgtaagaagatcctctttttaagatttctccgcaggtattgattacatctctttttcttgtttggtttagttagttacactctagtttccaaattactttgttccaagtttctttagacatttagcttgtaacagaaatctgttagaagatttctagatcagtagttgtctagatcttttgctgccaaggttacaaatgtataccttttcttttatcaggagtgtgtcatttggatccagagtgttttacccaatataatattcttttttaaaatttatttttattttttatagagatggggaaatccctgtgttgcccaggcttgtcttgaactcctgggctcaagcgatcttcccacctcagcctcccgaagtgctgggattacaggcaagagccaccacgcccaccccaatatagtattcttggaaagttgtctataaaggatattttgatacatggaatcatgcaggtctttagttgcctgtgactcctagcaagtgtactctgccactccgagtagttaatgatctgtaaataagcactttaatgccctcttggggttgttttaaaggaactggcgaataaaggagaatcatttatacgtacctgtttctcaagtttgggaatgtgagtattgcatttttataaagaatgtcaaccctgattatactggatacattttattataatttagaactgaatagtaattcagaacacatgctaatgaggtgcgtcagaagcattcctgtcatgtgaagggcaacctacgtgcatagaccagaattttttctagcctgaaatctgagaatccccagttgaagacaaattcttaccacaaattttccttttgattattgtcaatactatcagtgcctattgtgtagtaagcagttaataaaaattcacacagaattgcagttgcctttccttttctgattccatattttcctttgactcactcaagtagttcttttgtcccactgttgcttggctgttacataagacgcctttaacttcccgtttctgttcctaaaactgtgagtagtagtacaacataaggtaagcactgatttttggagtcagttttacctttagtctctaccatctaatagttgtgggacattcaacaagtgacgtccttttctcatctgtaaaatggggaacaatgatagtgctatctatatacatggttgagagggttagcatttagtaaatagtaattagtggggtttttttgtacctagagtgaccatatatcctggtttgcctggcgcaattctggtttatacctgttatcctagcatgcagtccaaggagtgcctccgtagctctgaaagttaccctggtttatgtggccaccctatttataactctctggatgatgcaacatgatagtgcaaatgagagcctaattttgaaatgttaaagagccaaaacaaagaagtttctattggaatgtcttttcaataagaaaatttttttctgacaaaaatgttattttatatagagcttacagctactgaaccccaaaacattttgttaactacaggggtggggtgtgtagacagagatcttagagcttttgatggagttgagatgtcctctaccttaccattccccattcatcccccgtaatacataatgcactcatagaatgtgggtatttgtcagacactgggccagtctcagtccaggagtttatttccttaaacttcaggtgtttggggttttcccaaactagaatccttgaattacatgaaataattgcaaagacttctgaactgtgactttatcattttaggtatgtactccagcacccccgcccccatccctgcaggtactgttttctcattgctcttaacagcctgcttctaattatgtataactgattttctcctgtagaaatgtgtggcctttgctgcatgccactcatagaactagagtggagcatccatgcctttgttgagagactcagactgctttcatttttgtttctatctctgtttaatttttctaaaggatttagcctggtgccttaatctcagaatgtagataatgatggatactgcttatcagtatacattggggggcttaatcattcactacccaaatggaacacaaagtaaaaagtttaatgaactattggtaaaaggtagtatttggaaatctggggtagagattggaaaagtacttttgagaaccacaggtacaagactctccaagcatgctcctgcttcttaccataaaagccccaacgcctccgctgatgtgcctgtgtacttaacctgtggggcttaatgggcttaacatgttcttctctaccagcccaagccagttcacttgttttaaagtggacagcctgtactcagttgtgtgatggaaatggggcatattaaacaggaggggtctgggactgctctctgttccatgctgttcatcttggacctgcctcaggcttacttcttcacaatttgaggaactgctcagcaagagcacaccctattgttgacatgtttttcttttctctccggaacctgcgagtgtacgatggcattagtggaatccctctgagcctgtgtgccaaggaaaatgctaatgtgctgatccacaatgatgtgtggctcagtgtgaactacgtagtaaatttgtgagtgacagagtaaaggggcaagaggctggaggagatgagtataaccatctttcattcacttagacttcctgaatgaagccgttcatactgagttttgagcaatgagaggattttggaagttctgtttttaataaatggctccaactcttctagtacttactaattgtattaaggtgaacctttgggacataggtttccatatgcttaatgggaaatactttgtcccttttcccccaagactggcttgacctcacgtcaagacctcacgtcaaagcttttgggccatgacctgtttcctttgtggttccagaaatctgggggcggggggaagaaaagagagagacctaccataaaagttaataggacaggctgggcccagtggctcacgcctgtaatcccagcactttgggaggccgaggcaggaggatcacaaggtcaggagatcgagaccatcatggccaacatggtgaaaccccatctctactaaaaacacaaaaaaaattagctgggtgtggcagcacacacctgtagtcccagctacccgggaggctgaggcaggagaatcgcttgaacccaggaggtggaggttgcagtgagccaagatcgcaccactgtactccagcctggcaacagagcaagactccgtctcaaaaaaaaaaaaaaaaaaaaaagttaaaggacagagattctgatctgccctctttgaccagacccaccccttctcctttctcaaagactttaaggagaagacttcaggttctcatgactgttagctttattttggggatgtagaaaatagagattcactttgaattttcaaaatgcacatccccatctttgtttctaattttagaagagaggtcagtaagtagacagatttgtgtagaagatagagaattggaatatgcttctcttttgcctatttgagggaatgctcttgagaatcactggattagagactgtagaacaatggttcttactctttgagtcagaggtccttttaagagtttaatgaaaatcctggttctttcctcaggaaaattcaggagcacaaaaatatgcgtattctgttagaggatttatgggacttctgaaactcatggatcctgggttttaaagtcctgatgtggaaggccaggcctccaagccacagcatctcttttcattacaagtccacaaactgctaagagatttaagtttccagcctgcagtttgtccttatggcttggaacaaagctgaggttgttggcattgtcagctccctctttgggatgctcctcagcttcccaaaagactcattataagtgccttgagagttcagctcaggaacttaacacacattcgaaccggcatttcggccactttgggggaatttttggtattatctcaactctactgctcttcatatgaaatgcagatgcccctgaagctccagagctgattccgtaggagtgtttgaaatagtagctcacatagtagattatcaaaactaaacaaaataatttgcttctgtatgtctctcaaaaataggttggatttcaatgtagtacttctcaaactattatattttaaggactgacctgatatttgaattttactcatcatttgacttttctagatggaatatgttgagggcaaagaccgtatctcacacatatttatatagtacctggcacataatactgtcaataaatgcttattgaataaaataatgtgagataatcattccttggagaaagtaagttggacttatgtggtggctttgactcactttccctttcagttggctggggtgatttttctaacttagagccctctcacataggtataaggaagtaccaagaactgcaggtgagaaattgatgagaatttccaacttaatagatccctttataaagccagaaataggaggtcagggagaagacatctgaatatatgcctccagcttcttagcccagtggcagtgggcatagggatatccttagacaataatggggaaggtaacttgaatttctcaccattgaaacaacttttaaacccttagcattagggagactctgtccaaagttgaaactggtatcctggtttatacatgctcttgatttaaagaacagggaggctttcacatagggtatttgaggtttggggattagcgatgagttgaatagtttgattttggaaagtacattggaactctgaaagtaataaacaacagctccgtccggcagtgatgagcaaacttctggttcttactctgcaaccctggctgccccttctgagtcaaaggctgagtttgatgctcaaaagctatacatgcccttctctctttgaaacacaccttctttagaattacagtcttctgggaactgggccaatgcttatcctagccagaagctcaggctctagttggcactacattttgtgtgttctgtttcacttcaagtgtataacttaggcaagattcaggggaaaaggagaccttcagaattttgctattttgtgttgtttgtttgtttgtttgagatggatttttgctcttgtcgcccaggctggagtgtgatggcacgatctcggctcactgcaatctccacctcccgggttcaaacagttctcctgcctcagcctcccaagtagctgggattacaggcatgtgccaccacacctggctaaattttttttggtgttttttttgtttttgtttttgtttttagtagagatgggatttcatcatgttggttagggtggtctcgaactcctgacctcaggtgatccacccgccatggcctcccaaagtgttgggattacaggcatgagccactgcacccggcaaattttgctattttggaagttgaaataggtgcttcctagagatagcagatgtatccagttgctttggtaactaattcagtttcttatgggtggccaggtggtatcccacttattattcaatacttgagggttcagtgcccagcttctgagcaccagctttcttagcaagcaagacatgaagattcccagtgttcatttatgagaagtagcccagctggcttgtcatgctggactagatgaattgtgaatgttaattatattgataatacagtggccttggtaacagacttgtagctgctgtagaatctgtttacttcaggtttgtaacagaaaagagaccaagctggttgggaacatggtgttctcagtgtaataagatgtattgtaagtccaaggaaaatgcatcttggtggggctgaccttattgcaccccttccccaagatttcccggagagaaacattattctggggatagtttctgttcttgtttgccaatcaataatgaattgaggttggcaaaggctgggtttttagtggaagtggggtaaagcatgtttacaaatcataaagtttagaaggagaacacctgtgttaagcaccttatttctctacctagtttataattaggaggggatggttgatgagattagtttcggcccttgcattcaggaggtggagcccctcctatttggggagcagggggttcataattctgtacattcttttcttactccgcagcgaggggattagctgagcactgggcatgtctggaatgagagcactttgccaatttttactgaatttggaccaaactaataaaaatacaatctcggtcttcctcagacagtctctttgtcatgttctcctaacccctaggatccatctgatggattgtctgttcctaacagatggagttggcattaaaggtagattctctacttcctttgccttattgttttatttctgaacaaggcatatctaatgcatttgttccctgtattggaagttgctttatcaagagcctagtaaactcagtgtacttgtgtttccttttcagatcctgcttagattatttcttagatcagcacttctcattctgtggaaggagaagaaggtatcagtcacatagagggttttttcgcaataactcattacctggctctaaactagagctgtgtaatcagaatgggggaggagctgagagtaagagaacagatggagaatgggttggcattttgaaacagcttcaccagataaacttgaaatctcccagatttagaaccattcttttagattatctagcaagactggccaacattgttcggaaatctaacattgagatggtaatggcccaggcacggtggctcacgcctgtaatcccaacactttgggaggccaaggcaggcagatcacctcaggtcaggagttcgagaccagcctggccaacatctctactaaaaatacaaaaattagccgggtgtggtagcaggtgcctgtagtcccagctacacagaaggctgaggcaggaaaatcgcttgaacccatggggcggagcaggttgcagtgagccgagatggcatgactacactccagcctgggcaacagagcaagactatgtctcaaaaaagaaaaaaagatggtaatgcagctaccaactctcctggatgattttgatcacgaggagaaatcttggtcctccctcatgtgaaaggtcctgaaaatagtctaccgttgctctttttctcaatgtattcagagtaaaccatgaccaggaatcatgtagccccatctgctcagggtgcagcgtcccagggcctgtgctaggcacttagggtgcatcagtgaacaaaacaaagattcatactctgtagagctgacattcttgtgaggttattatttggatcatttaagtccagtgaagtggttcttaaccattgggggtttggaaccctctgagagatgtgtacgtattccagcaaaatgcacacagtcatgtgtcacttaacaagggggatacattctgtaaactgtgcccttaggcaattttgttgttgtgcaaacgtcagagagactttaaatgactggcaacgcagtaggtttgttacaccagcatcaccacaaacatgtgagtaatgcactgcactacgatgtcaccaggcgataggaatttttcagctctatcatattcttatggaaccaccactgtatatgtggtccatcattgaccaaccttcattacacagcacaggaccttgtaagggggaattggcagcctgcaacccatccatggacccagggctaagaacctagaagacttgtctagatttgaggatgtggtgattgggataaagccaggccctgaattgggtgtttggaaccattatctcctcgccagactgggttgtagtttcttgatctgtacagccaaagcagtatgtaaacaactcaaggatatcactagaaaaagagggaatggctaagccttctctaagctaaagagtctatgttttatgtgaaatgctaagaatgattctgactctgttctccctcttaccctagtttctagttggctgggcttctggtgctcaatagattaaaagtcaagtcagaataagaatttccctaatgtgctaatttatgttatgaattcctagactgagagtgatagtaaagttataaagatcatgaggacattttagtgaaattctggcttcaaaaacacagaaagtgacaggagttgaagggctgttgaaatagccagagtagagattagagaaagatgacaggaagaaaaggagggcagaactcagacagtcaactaagctggtgagccagtagccaggagagcagctttctccttaagccaaagtgagcagatctgagactcatgcagaaaaatactcctttttggtcttgaaggggtctgtcactgttgctttgttaccaaatagtacccgggataaaaagcgggacggcagataagggtttgccttaacatttataccaacgttaatatatctctgttgctggaaaagttggtgttaaacttgaaagaatggccaaaccctgcctgagggttagtgcagtgtgtaacgcgtactgcagaaacatgaatgaaatgggacactgggagagagaattatgtagaaagacacagccggatttagaggttggagcacatagagtcctgtgctagttccacctttacctccctgaaatctgatctttagtataaccacaggtcaagacctctttactttaggttgatctttacagggatatctaagggaaactttccatatgcacactggaagatagctgaataagggctgtgttcaggctaaacttgtataaactcatctcattagagaacagagcccacagtcctccccagaggagtgaggaggctctagatggttccagttataactgctctccagcatatggaaaaactgttctgctatgaaaccttccaaccagctactcacataaagacatttcattgtttgctcagtgatgcattaaagctgaggctgcctaagctagagaatataatcacataccaattatctttattttttatttttaaagaagccctttaaaataaccaaaagaatttcacacctcacttcctgggactgggactgttttgcagttgtattcagcttatgaagttatatagttaaaaattgctactaagttttcggaaaaattattcttaaaaattataattaaaaattcttattcttgggccaggtgtggtggctcttgcctgtaatgctagcactttgggaggccaaagcaggaggatcccctgagcccaggagtttgaggtcagcctgggcaacacaaggagaccctgtctctaaaaaataaaaataaaataaataaaaattattatttttttgagacggagtttcactcattcgcccaggctggagtgcagtggtgtgatctcacctcactgcaacctccgtcccctgggttcaagcaatcctcttgcctcagcctcctgagtagctgggattataggtgcctaccaccacacctggctaatttttgtatttttagtacagatgaggtttcgccatgttggccaggctggtctcgaacttctgacctcaggtgatccacccaccttggcctcccaaagtgctaggactacaggtgtgggccaccgagcctggccataaaaattcttaagtggaaaattttctccgttcattaattgagtatttgatatttttttcagctttattaaggtataattgacaagtgaaaactgtatgtatttggaccaggagtgatggctcatgcctttgggaggctgaggtgggaagattgctcaaggcgaagagttcaagactagctagggcaacatagtgaagtccccatcttttacaaaaaagaaaaaaaagattagctgggcacggtggcacatgactgttatcctagccactcaggaggctgaggctggatgattgcttgagcccagaatttggggttgcagtgagctatgatcctgccactttactttagtcttggtgacagtgagactctgtctctttaaaaaaaaaaaaagtattcaaggtgtacagtgtgatatttttatgtatacattgtgaaattattaccacagtcaagctagttaatatatccatcacctcatagttactttgtgtatatgtgtgtgtattgtatgtgatgagaatacttaggatctactcttagcagatttctagtatacaatgttattgttaactgtagttactgtgctattcactaactctccagaacttatttatcttataactccaaactgtaccctttaaccaacatttcctcatttcttccaccttcctgacgcctggtaagaaccttctgctttttgcttccatgagtttggcttttttagattgcacataaaagtgagattatttaattgattactttaaataaatatctaccataactatgaaaccaagattcagtaatcagatatcatgttacttaaatccagaatacttcaataaaaataatagccttacaagaacttttgttagttgtagtagtcagaaaggtggtaccagaaagtagaaatacatcaatggtaaaattttttttttttttaacctgaggtcttcaaatttaatacaacatttattttatttttatttttattttttggagacagggtcttgctctgttgccctggagtacactggcacagtcacagctcactgcagcctcgacctcccaggcttggtttataaatgtgagccactgcacctggccaaatataactctttttctgttggtggaattcagctgtgctagatcttttagacataagacttggaaagggtgctttttgagtggcacaccacagatgagaaaggaacctaatggaatgtttttagtttaaaagaaattatctgattgtgctaagtgttcatgtgtgaaagaaatattcctccagcaccattcagcacaggataaacctcttcaaaaagcacatttaaagtatattctctgttgcatgttttagaatatggtaacttctcatgaaataaccagcgaatttccccagtgaccaaaagaggaaaaagaaaaaatcagtggccaatttgaccatactaaactaaaaattttgcttaaacgcaggtggaaatcatacaaatagggaattctgaattaatttcttttgtcctagtaaagtagtctagattagattgcccatacattctcctccttactccatccccagaagacaatggaagtgtattctagaatggaacattttgttaccacagtgtttgggctctgctattactgggctctcctcaccctttccagatctatggagcagtacttttcaatggcaatttgttggcaacgtaaaaacattacatttctttgcatcattttacttttctgaaaattaataaaggtttttggaacttaaagaattattttcctttaccagtgcttacaaatctttatctggtgttataagctcagaaagcttatagtgcaacagtagaccactccaaggtaaactgaccccttggaaataaaggggaaagctgctgtttccccagcctattccaaaagaaggaaaatagaactgggcttatgggtgttttttgtttttgttcttttttttttttgctttttgctttttaaacctcagtgttggtagcaaattgccttaagcaagtcacttagccccaacccctgttctgttttctcatttgtgaagcatagatgcaacattgaaaggattctagaatgttttgaaaatacaaaccctctgtaaactgtaagccacaggtgttgaggcaaagcacttaaagtcatataaatctaggcattcaggatcttggtgtccaccctactagtaagtgggatttcaaacttcctgtacaatcaactttaagtaaaaatcttcatgtagtgctatcaggtggtgttaccagtgatgcttgttctttgtatgccaagagttcaacaaaaatggtttgtaaaatatttagatcagtgatattctagcagtgatatatattgacaaaatttgccaatattttgctatacaaaattaattttttttttttttgagacggagtctcgctctgttgcccaggcgggagtgcagtggcatgatctcagctcactgcaacctctgcctcccaggttcaagcgattctcctgcctcagcctcctgagtagctgggactacaggcacacgccaccatgcccggctaatttttgtaattttagtagagatgggtttcaccatattggtcaggctggtctcaaactcctgaccttaggtgatccaccctgctcgacctcccaaagtgctgggattacaggcgtgagccaccacacccggccttgacagctttttatttccctgagcctatactagttggttcttataccctatgaaccaatacaaaatgattcccaagtgaaaaaaacaaggtgcagactatattatcatgctaccttttgagtaagggagggaaaataatatgtattcgcttcccacactcctaaaaagaaacataaaaaggataaaccagaaaccagtgaaaatggggagggacacgtagagtatcagggctaggaatagcagtgaaacttctctgagtgtaccttttaaagttttgagacatgtaaatgttttatatgtcaaaatcaaagagaaaaaaggaaagctttaagttcttacaaacacaaaggaatgaacccaactaaaatataaaatcttaacctaatatatcaaattgattactcagtcacacagaaaatagtactttgattatatatctttatgaaatatattctgaggacgagaagaacttcaatgaaaccttgacacgtcactcagtaaattttggtgcgtaataacattagtcttgtcattttgaaacttttgtatgcattatagaataaacaaacattagagacaagacatacaataggaaaattaaactttttttaaaaaaggtaatactaaatctgaagtagaaatatcaatatgaactcataagtaaaaggattgatttctctctctaccaaaagaacctaggagtagtgcatctctaatagcaattatcacacctcttactccaggtattagcttctaaatactactcccttctaaaagaaaggctcctgagagaaatggttgattccaggtttggagaagaggaagaacaaggtgagcctggtatatcttcttttgtcaaaaagagtggaaggactgtaatggaatcatgtcaaaggacagaagagccagcttgaaggaggctcccactggcccaatttgagacaatatgagtattcagtgcaattaatgtataattaagtgtaatctgttaaatacataaaaatctgaatgacaaagggagaagatttttctttacaaaagaatgccagctaatgtggaagcattgatagaaaaatagaagttgcgattctataaccttcccaatataattggttcaggcaaggattattaacagatggtgaagtcattagggaataggatattaacatgatgaaaaaagtattgccctacaaattaacttttacttgcaaaagagaaaatgtacctttcaaagaagagatctgatgttctcactttaaccaggactcaatcttagcattgctaatagtggaacaacccgacatgtgcctcctgataatgatgcaaggtgaaatatactacataatgcaggaaatattcttgccagaattttaacctttttcaaataaaacctcagaactgaacttccagtttataggaaggaaaccctctgaaaaattcaaaacatgagtgattttataagacaattagtctggattcctcaaagaaaagtcagtatcacaagggggtgggagacattggagagactgttctagattagcagaggctaaaggaacctaacaaccaactgcatttaatgaaccttgaatccatgctgggttggcagggttgaggagactgttgggatataaataaatattcttgagaaaattggggaaatttgaatataatctggacagtctggatatatgaatatatagaataattgttaatattaagtgtgatatgtcatcatggttctgtaggagaatattcttattcttagaagatacattctgaacaatttattggtaatgttcatgtttgcaacctttatttttacaggaccatgtgaaagtatgcatgtgtatgtgtatatatatagagagagaaaaaacctattatggaaaacggaaagatttattctaggtggagagtacaggtgtttattatactatgcttttgaatttttttatgtgctgaaaatgattataataaagttggaaaagtaaagtacaacaatggtgctagcgagtggggatggagggtagggggtgggagaggagactttaatgttgtcactaaagtagtgggtatgttccaggaaccaggatttcttcagtcacctgtactgatttttttttttaaacctagaattgatgataaatcaatatcgggagaaacagagatgactggcatgtgtttatataatattggtggtgggactgagaaaagaagtatggacaggtcaggcaactaggtaggtagtagatagctaaataggcattgaaacagtctgggtgtgtgttacattctgttgcaaattgtggtgttcaaaagattgggaagcccttttctgtcctacttactgccttttttctaacttcttatgtttacttctagttggcagttgcaggcctcgattactcattgactggagcactttaggagataatagtgaagtgaagtcagtgaagtgtttagtcttacaggaagagaggggagtagctcacaagggtcaaactactctttggcagagtttttcaagctgtgtgttccttaccttttaagtgactcagcagtttggggcctggtattgcctcacctcttctcgccgcgagcattgacagaagatctagttgagtcctactttgaaaggagtattgcttctgactgtggaattggattctagctgtgtgtgtgttatatcctgttactgttgagacacagaggcgggagtcggcgggggaagatatgtgctgagattagtcagcctgccctccctctcccccaagaaactcaggccccatctggggagagtgagaacttaagaaacttgagacagggaaggagagccgggagcagccaaaacctgctaagtctcagaagactggtttctgcacaagggaatggaagaagtgactggaggtggctgtggattcaactctggcctgagattgattcccttgagataagttgttttctggtgggtgtgtctgggagcattgtgttgggaagggaattcctttctttgggaaacacttttttttttacttgaagtgaaaggtattcagacttgtgtatttcttgtaccaaggagtgggtgtttattgataactgtttctttgggttcatctgaagtgggaatcagactggtaaatatcaagaaaaactgggcatggtggtgtgggcctatagtcctagctactcaggaggtgggaagattgtttgagcccagcagtttgcagccagcctggcaaacatagtgagaccctgtctctttaaaaagagagagaacgtaaggaagtaaaggtgttacacctgttttggctgtctgccatattctcttcctttcctctgaaaaaggagtgggtgagggtagaatctgtagaagaaaaaataaattaaccagaaaatatgtgtgaaaaagggattagaaaagatgtaggaatcttgaaaagtgttctgacacactatgcttagtaatgatgatcttgttaaaatatctagttttttattgttactttttttttgctcagttgctaagaaatatgctgaccataatttctaaacagaaaaatctctgcacacaatatgctaggacagacaatgggacagaatatataaatttaggaatgtcccagagaatcagtagtgtaccaaaagagggatcctagcccttcaaataattctctgcagtaagaaagactgctctgctcacttctacattacactttcttaatgatttgttttcctagtaattgttgtttatctttgaatctagttcttgacacttaggaagaaacctttttgaggaaattaaaagctgttagattggtatttgaaataacgaatttgacaagatgtctaagttaaacctaagttaaacaatatccagataaggctgtgaagactataatatctggcatgttcctcatggagcagtgaaattccatgccaaagccacttacattactatactgtgggtcaaaacatgtctacagattttaagggaccgtgtttccttgcatagaactttcctggagtagcactggatacttagttggtgcctagcaaatgtgatgactaaagaatataatgcatcacagatattatcctgatcaccatttcttggctaaaatgttgtagtggaattctggtggctgttaattctttctcattcaggagatagcaggcaaaattgggtgggccctgggaaggagtgtagctgggctatttaacatctgagaatataggatgtgtgtttatgaaagtaaactcagcttctaatggtaaaaaaaaaaaaaatcaattaggatcaactaaaggaatagcatacaatttttcttctcttgaggtatgtaacttttttggattgtctctgatccccttctcttggtaaggtctgcacaattctttcagctcactttgtcagcagacttcccactaggtagtgcatataccccaaattattcgtttggtagagcttttctttaccattctcagtatcacatggaagaaagtcaaccattgtacctgctaagctatttgaagaggtgctttaaatagcatgttaaactggcctgatatttaattgggtttttaagacacagcaagtagacttctgtatttgggtgtgctatttgaagaatgggtcaatctgaccttctagcctttcataaatatgctcttagtgtaagaatggaacactggtatgcttgttcagttgtgggtaaggctgtttatggggtggtgagttactcaagcataacatcattctttaacataagactttttacttaaagatactttctgcatttcagccactttatttcctttccattccttctcttcccaatcctactttttacttaggtgtagtattccttgtagatgtagatttgaagaaaggtgaaagcacagtaattaagacttaatgtttggaagaaattatgatgataggaattaatagaaacagaacaactttgtagtcttccttttcgtcttacccagattatcatatttgccttaaatgagtgagtccaacccaatcagaaggctgttgttgagatctggagagaaaacttgtgatatccaaaagttgcttttggcagcagtgtttatattttcaacgtgagggtgaacaatgctccaggagatacttcaccaagagaaaacagagctactagctgtgaagataatcagtggagctagcaggtctggaggaaactggcagagcagatgtattccacagcatcagttgtaacacacattctcttcaggctgtaatttggggtggaagaaaagaccaggttttcatgcatctttgtattgctgggacaccttataagtggtgagaatgtcaaacagctgcaagaatacacagtccattcatccttgccatggcttgggttaactacattctacccacttcaaatatggaccatattcattatttgagagatggggaggtaggggcactgcactggttagtaagctgctaagctgcttgcaacacagtccagtatttttagatgggaaagattttgcttaggtttaggctactgatgagctttattaatataaggggaaattatgactattcggtttttaagttgctgaagaaacacatttctctcttgccacagccacctttctatatgaaaaactgcaagtgttattgcagttccctcccccaccctcaccctccatgtacctggtatctgggaacatgcccagaaccctttgaaaaaaggcgaacaggggaaggaatcatagcagaggggagagtagcttccaaatagaataggctgcatatgccagcagcagggaggctggtggaggaagggctgcctctggagagtgttaagaggggttgatggtctcttgactgtgtatttttttccatcatcaaaataacagcatattgagttggaagtcattaccttggtcctggattaatttcttcagcaaaactagctttactgactgtagaaatcagtttccaatttgtttatattagctgcttgtgcctaggctgcccgcttaaacctgattgctgcactgcaatctggtgctcccctagctgaagggtacatgtgaaagaggaggagaagcttttgcagctgtaactaatgggtagtttactgttcctggctttaaagtggctgtaaccatgttgccagtattatacagagaagattttaatctgtagtcatttacatttccagagtatttgctaaagacttagcttaatggattctccaagggaaccttttcagatttagtcagatcgtgaacagaatctcagttcacatactcaaaccatcctgcttatttcaggggtgaagtttattttattttgaagaaagggtttatcagagtttaaacaggagttcgggaggaaggtttaaaccatgtaataaaagtgttacacactttataaatctataaataatatcaattgcaagctatttgctattgattagagccagttttattcttcaaagaattatctagttctggcttatttcatttaaggaagttttttttttgggttttttttttttttttttgagacagagtctcactctgtcacccaggctggagtgcagtggcacgatctcggctcactgcaaccaccacctcccgggttcaagcaattccctgcctcaggctcccaagtagctgggactacaggcacccgccaccaagcccagctaatttttgtatttttagtagagacggggcttcaccgtcttggccaggctggtctccatctcctgacctcgtgatctacccaaagtgttgggattacaggcgtgagccactgtacctggcccatttaaggaagtttttaatacaaagtttaccaagtcattattttctccttccttcagattagtcctaatgagttagaatttactatagtgtatgtgggtttttaattttctttttatcattatattctggtattttaaatttttaataggataggaggttctagagatattacattggctttgtgaagtatagacatatggttgggctctgagtattggcaccctaatgcccatcaccatttctaggcatctcttcataattctttcagaaagcaagaccatttgccaactttaggaatgtgctacaagggaatattctgaaacaaaaaccaaaaaaccccagtagcactatgagattctgttttctgaaagatttgggaagaagggtaaagagtttaatctgtcagggttataagaggggaaaaaaacttgatggaatgtgagaaagatttataaaattgaacatatactaaacgaaattggttcttaaccttagcccctcctcccaaatcctgcagaaacaattgggaagagacagtgattgacgtgttgggagaagtcagagagtagcctcggccttcccccatttctggtgggaaagttgctggcttagtaggaaccatgtgattacatggcttttagggaaagacaatgtatccttaaaaaaaaaaacatttcatggattttccttataagttttcaactttttgtcttccttttgtatagcaaatccagtttgctgatgacatgcaggagttcaccaaattccccaccaaaactggccgaagatctttgtctcgctcgatctcacagtcctccactgacagctacagttcaggtaggtgtgaatgaactccatgtcctctctaatctctcttccctgtttgctaccagtgctcttcacttagcaatctgaaaatacctcatgttatgactgcccacctttgtgctgaaaactgttagtggctgggcagtgggaatgttctgcagccaagctagtagctggggaagcttttgtcgactcgagactttcttccccctggctgtctgtgtgtgtggtctgtggccagtgcccagttggtaggactgggctagttcccattcccacattgatccgcccatttcaaaaggaagaaatcaatgctattttggctgacagaattggacttaaaaaagaagtgcattttgaagcacgatttctttgtgtaaaatataccctgtcacctatctctttctttcttgtctccaagactgcttagattgtaacaataaaattagtattttggcttctcttatttattaggttattctttctgttggaatatcctcccctgttagactctggagcaggaaagattcagactactgtaatgtaactccagctctgtttctttttagttctctcctcctgtcttaactctctgtaaataaagtctttttctgttttgtattgtgtctttcccaactatagagcatgtattagaaaattagcatggaaaagaatatagaatcttggcagatgaatacaggagaaaacattagggacatctaatggggcatccaatacaaccttgtcattgtttaactgaagactcattgttaaaagattaagaggttacagattgctattaatgtcagcagagatatttgtaccaaaagccattcattgtcctttgccagaataccttttgcattgtattatcaacattaaggattaacataaatgaaaagctgttatatatgatcctggagtgacagataaaaagtggtgtgtgacaagcaggctaactgtagccaagaaagaagaaaaattaattgtgaaaaattgagttatgagaggaagaacgataaaacgactgaagaaggcttttaaaaagtcttcaaagatcttgacggccatgttacagaaatgtattagagccttttaaaaagcttagtgatatggttaaaatgtcatagtggggaagataatctttagtagtatttgtgttagctggacaggaggaggacctgtacatagaagtcagtcagtgagagagtccaggtatactagcagaaaccgtgatgaggaaagtactcaattcagagaggaaagaagtgatttgagaggtaaggaatgattgttgtatatcctcttgcatatctgtcagataagtggtatttttagcttttatgtgaaaccactttttaaagttgatcattttgacagactttcccagggctatcagtagaaccagagctgtttgccttttgtgccatttctttctttctctattccttgagcacaacttagtgtaaaatcccttgtaagactatcttatcacttttgtctatatccatggttctcaaagtgtgatcctctgactagcagcatcagcaacacctggtaacttgttggaactactaatgcttaggtgccactccatacctgccagatgggaaactggccacagggtctagcaatatatgttttaacaaaccttccatgtgatcctgatagatgcttgaatttgagaaccagtgatctatagaaatagcattccttatctgccctaaattattcttctgtttttgccaggtctggttccaatccagatgggaccaagtacctctattatctgggattactatagctgcaggtttataatcagtggtcccttgtcataggcactaatagcacagtattgttagggtgggtgaaagagctgcaacatggaagtacccacaacagtttcattcataccacctattagaacctagaggagcagaactgagagcgttgtaaaaggtagctccaattaagggtgttcccttaacttaaatgaaagtcccttggggactgcactctgtagagttggggaccactgaggtttttctatccttgtttttttctttcctggctctagctgcatcctacacagatagctctgatgatgaggtttctccccgagagaagcagcaaaccaactccaagggcagcagcaatttctgtgtgaagaacatcaagcaggcagaatttggacgccgggagattgagattgcagagcaaggtaaagaaagggagtgggttaggggaccagaaacaaccctcttgttggccatcagaatccacaaacaacttaagacttgaaagctgacttgaaagtgtactgggaagaaagacagtattatggactttcggataaacacttctctctctatggaacttaagtcttgggattttgttctctcttccttctgttctcagcatgtggtgattacacacagagtagtttctgttttcctccttgccccaccccaccactgtattcaaagatctacatggcagaattgacaagtcctagatcactacgtggtagttggttatatgggatcttactatagctgccaatggcagagacccagattgcccagaagcagctgatgaggaagggttttaaggtaaggatgtggtgacctagagtcttgtttggtcctgagcaccctctgctggagcagagtaaaagtgactgacagttaaatcttggtttctctaccccagccactcccagtaaggtcctctttactcttgtgttgtttacacattgaaggcatgttctatactgagagagaatagagaaaccaaacagactgacaggacatggtaagacagcatagaaaaacagaagttagtggtttcggctgagggtacagtctttctggtctgatgattatatataggaatctattagccaatgccttaactattttcctctcttcccaaatgaatgatgagactcacccttcattttgatcagttttaaggatttacttcattctctgttcagtcctctgtgtataaagctgaagcagtagaggaagagagaaactccaagtggttgcatgactgctctccactattactgtgccgttgaagctgccagtcgtcatggaaataggcttaaagtttttctgcctcatctcaccatatctcaacagatccagaccagccctggggcaggaactgatgatactgctagtgcagacctagctcaaatcctctgttctttcacagacatgtctgctctgatttcactcaggaaacgtgctcagggggagaagcccttggctggtgctaaaatagtgggctgtacacacatcacagcccagacagcggtgagtttgttggaagaaaagagggacttctgataaggggaagaaagaaatcaattttttttttttcacttttcctttcctaactcgccaacctccaaatgctaactattaataggatctccctgttacacaataatggggttttctgggtttttttttaatatgtaaacccactttcagcaaacttgacctaggaaagtctagatttgcaaacatatgattgttataaaatgttataaagacttttagaaacctatgttttatgcaaagttaaataccagtgcaccacctcgctggagtacacccttttgtaactttccagtgtagcacctgttggtgcagagttgataggtattgctatcttgatgaggcttgctctccattcctgggtggcccttctcttcctcaccctgtcttcctacacttctacacaggtgttgattgagacactctgtgccctgggggctcagtgccgctggtctgcttgtaacatctactcaactcagaatgaagtagctgcagcactggctgaggctggtaagttcggttttttcccaccaactttgccccaaaatagttatccaaacatataacttttttgtatcaaaattgtcattacaatatcatatatgtctagaggctataaaattgtgcatgataaaaacctcaacaggaagaaagtggataataaaattaagatggatatccatggtggaatattatgtagtcattaaaaatggcctttgaaaataatctaatggcatggggtcatgcccagttctagtgttagttgaacgaggaagaatgttaaactatataagtgccatcaattttgtgagagagcacaagtaagaagtacctttcatattgatagtgcattaaataatttggtattcagtatggcattgaaaatatatgagctatttgtgagtttaattaaacgtaaaataattttttattattcaggccaggcctggtggcccacgcctataatcccagcactttgggaggccgaggcgggcagatcacttgaggccaagagttcaagaacggtctggccaacatggtgaaaccccacctctactaaaaatacaaaagttagccgggagtggtggtgtgcgcctgtaatctcagctactcaggaggctgaggtaggagaaccacttgaacctgggaggcagaggctgcagtgagccgagatcatgccactgcactgcagcctgggcgacagagtgaaactccatctcaaaaattaataacaataataataatttttattattcaaatatgtcagaagaaaatcaccgtgaagtttctttgaggacctctcatctttctttctgttttttttgttcttaagatggagtcttactctgtgtgcccaggctggagtgcagtggtgcaatctcggctcactgcaacctccgccttccgggttcaagcgattctcccgcctcagcctcctgagtagctgggattacaggtggacgccaccatgcccagctaatttttctatttttttagtagagacagggtttcaccatattgactaggctggtctcaaactcctgaccttgtgatccatccgccttggcctcccaaagtgctgggattacaggcgtgagacaccacacccagccggacctctcatctttttaagatcctcagaaagcaactttataatacttttgggtttttttgtttattcttttctgataagtgttcactgcaaaaaatttaggaagttccccaaaaccctacaatcccactacatagagtatttgattaatgtttttccttttcctttcttctactttacataaattctaatatgtatacagttttataacctaaatacaatattttcagacgtagttttccatgtcactgaaaactttgacagcatcatttgtaatagtcattcatatggctatgtgatttcacctcactattttcctaatggagaacatttaggttgtttcaagatgtttactaatatcaataatgctccagttaacaacttttattaattcccacttcattttatctccttctaagtataaaattagtccagagaaaggaacactttaaggttccaaactacttttaaagcaaaaattccttaatttcttaacctaattaatttctgaaccaaaagtgattctttgacttttggctctgaattttttcccttctgaaattgtttcttttgcctttttctcttcacatggatctcagaaagtgatttggtacaggacactcctcaaaggaggaatcagctgattcatttctgtctctacaggagttgcagtgttcgcttggaagggcgagtcagaagatgacttctggtggtgtattgaccgctgtgtgaacatggatgggtggcaggccaacatggtaataatgcatatacatcctacactttgacaatagatttatttcctttttttccctcaaagcatggttccctaaatatgttttggatgtttggtcttatgcactgactttgttgctaaagtgtttagcttgatacctattcagaatgggtctgagtactgaatggaaaggtgggggaagggtaagactagctgaacacatggctcaaacacaccagaacaaaatagtggtgcttctttggagtaggattctgctttctttccttcctgccccagttctccttttgcattcaaaggggctatatatttcaagactctagactaaaaatgagtctgtgattagcttaatactcatcactgtagcctaaagcaaacctaggaatccttttaaccattttacaccaacaaatttgactgtatttccactctctaagcctgctctagggagctctgaggagtactcttccaaaacatacagggctctcttactagtacagaaagtgggttcaaagttccccccagccctagcagcctgaatgcccatagaagtcttctggctgttctatagatcctggatgatgggggagacttaacccactgggtttataagaagtatccaaacgtgtttaagaagatccgaggcattgtggaagagagcgtgactggtgttcacaggtaacagattctggagtagctgccccttgtgtccttgcctcagcaaactctcctggtctctgttaggctttaggactgtgtttcagacttataactaaatgggaatatcttcattagataaaattttgagtttttacttttgaaaaatcaagttgatcctagaaggaagttatttcaggttctagttcatacatttttctctcccatttactccattgagtgattctcaactagtgttgaggggcgggtagggaataaatgtatcttgaaaaactgtattcaatattgtattatcattttatatttgaaatatgaaaaacaatcctattgtaaaactacatgttatacacactacaaaaagtaaaactgggcaaaatattcttggcttgttggaatactgaaaagatatctgaaagagtgtttcttgggggcacattgcgcatgcttactcacacacacacggaaccatatcagaatcattgatagtctttgtttatcagaagtaggcatgggtttctattgaaaaatgctgatcaaaggaaataacactccctataaagggaagagaagtacatttcttcaattgaaaggcttttttttttcttccaaaaaaggggtaaagggagaagaaaatgaaaagaagtgtggagggatgacaaggaaataatcatgttttctaatatccttgggcaataaatgagactgggaagggcagcatggaacttgaagagaattaggcagattttgccacaaattatgttgtgccagctaacttggtttttgtttgtttttgtgacctcttctctctttaggctgtatcagctctccaaagctgggaagctctgtgttccggccatgaacgtcaatgattctgttaccaaacagaagtttgataacttgtactgctgccgagaatccattttggatgggtaggttgaatgtatatatattcaaacttctaggggctctggtcttcctttggctttaaaagtttattagagggaccatttcatactgagctcaaccagcttccaattgatattctcatgggcattggcccacacttttaactttgtgaagagggactgagtttgagctattaacgtttgagttgagcccttgtctgtttccacagcctgaagaggaccacagatgtgatgtttggtgggaaacaagtggtggtgtgtggctatggtgaggtaaatagctgggtctcagtgtctctttctttttgtgtacttattagaaatattgggtaaaggaggcttgtgctgtcaatctagagtcactgatacaaggtttaaatttttttagataatgtatctcaaacaatagcaaaagccttttattcagatttatgaatagaaacatacaaaatgaattgagttttcagattttaccattttgccttgtggaatagatactagtatcatcttccccaattataacaataacctttaatgagacttaattcaagtgaatatatagcattttagccccacaagtgacttgcagtagaattaagggtggaatctgaaatggcatatagttgaatatctccaaactggaataggtcacatattttgttgtcttcagcaatgacccaaagaagttcctgagctacgagggtatccttgagtagattcttctcaagtaagagaataggaaattagaggttgcttagagtaagttgcttttgcttagtcttgtttccctgatgaaaactgctatgcaaagccccagtccttcctatgacatgtctgctaaattacctctctaactgaggttcacaacgtaatctgggtctgtttctgttctgcttgggattggagcgtctgtgcagctgctcaccagatgacaggctatttcatgaaggttaattcctgtctcacaaataacctacctaccctaaatatccatgacttaatgaacctaacgacttgacctttcttttgttctgcaggtaggcaagggctgctgtgctgctctcaaagctcttggagcaattgtctacattaccgaaatcgaccccatctgtgctctgcaggcctggtaagaacagagtgataatactattagattcactctaggtgctttatgttattgagagttcataattgagtatattaatcatttttgcagaaatcacctaggggaagagaaggctaggactgctctgttcttctcactctaaccctagggctctctgagagcctcttggcatttttccttcctagagggagtcttggggactctgaagaggccagtgtatatagtttagcagagcttatcactcaccattcctgtgaggtggcactggatccaactagaaggggatcttgtctcatttcatgaccatcttccctcccactgccttcttgctttactcatagtgttcctttcttttcttccagcatggatgggttcagggtggtaaagctaaatgaagtcatccggcaagtcgatgtcgtaataacttgcacaggtaaataacttgcatagagaagtgtttattcagaggctaaatcttgaaagtagtctgagtgactttcatacaaaggaggtgagacctctgttcagtataaaggtctcagaactagaataagctatcagaaccttaacatgttttccagagtaaaaaacttttttccctggtgactttttatactgcttgatctgaagaccagatagcctaaggagcggttatgcatgtcttctctcagaatgtctacatgttctttaatgccagcaagcctctggtctcctatgttctacctgaaagccagccagtacctttgttgcccggcatctctttccttaaccacccatcttttcttttaggaaataagaatgtagtgacacgggagcacttggatcgcatgaaaaacagttgtatcgtatgcaatatgggccactccaacacagaaatcgatgtggtaaggcttctctcatttgttgctaggcatttctctactacctttcattaataaccatagtcaactgtgctttcttcctttcagaccagcctccgcactccggagctgacgtgggagcgagtacgttctcaggtggaccatgtcatctggccagatggcaaacgagttgtcctcctggcagaggtacacacagagaaggaccctggcagagcagcacaagcagagtagttagatctatgaggggggaggggagggaaacaaatattaggcctatgtttccctgaattttatctttggagagagagatatttcacaaattggtccccaaaataaaaccactttaaaaaaatctactttttactaagtaacactatttcctaaccacagctgattctttaacctttcagactcagatggatttgcagaaaataggaaaaagcctagagtcctgatctggaactgtaattcttcctcttccaactgagtctagaggggtgtaaagtcctctcccgctttggcttgacttgtatgccattggaatctgagacagagctcatcccagggctctctcttacctttccagggtcgtctactcaatttgagctgctccacagttcccacctttgttctgtccatcacagccacaacacaggtatgtggcaaaatgcctgcttacactgcactgcaatttgtggaactgggcgtagatcagttccagtgagggtgtactgtactatgttctcatcatcctattcttttttgatgtaataaattgtggaaacaggtattcttaagtatagaaaagagtcactaagtcagtattcgttgaattggctgttttagttgagaatataatgttccagcttagttttatttgatttgttctttcgttaggaactaaacaaaaacaacaaatcctcttaggatgtgtgctgccctttagtataggcaaagtcctaggtactgtgactgtggtagatcactgtagtacttacccaaatgctgtttaacctggttttatttttatgtgcctgtctaagaaatatttttgtgctttctggccttcttttcccaccttaggctttggcactgatagaactctataatgcacccgaggggcgatacaagcaggatgtgtacttgcttcctaagaaaatgggtgagtgaaaaacagtggaaatctatgcagacagctgcatagtttggtaaaatgactgccatgaaaggcattggatttagagtcagagttccaattcctgttcctgtgcttactggttgtttgacctcaggacaagttctaatctctatgatcctcagtttcttgattggtaaaatgaaaataatactacatacattgtagaattgtgtgagaatcacagctaatgagtaacagtgcttaacacaatggtttagggagatgttcttactctaagtagccttatgaagttcagtaaacttcccagtgcagctgagtacatacagccttggaacaaaatgagagaaacaaacacaggttttattgtccattttctgtgaggatatgcaaaccatccaatctcttgtaagcaacccatgctgtgttcatgtgacataatacatcatctctagtcctttgcctttctgcacggtgtacccatctcccatatctccttggctagcccaaatccctgacttcttaatgctaaaatacttcttgctctcctctcctccaatagcttacttcagacttttttttcagtgtataattataaaagggaagcttatctatggaaaagtaaaaattctgtgccgactaccttagaaattcatgatcctaagatccatggatcttatcctaaatgagtgttattgacacagggtctttctagcttactggtgtccactttgattgctaatttgactataatctagttaaggaggttgctgcaggaaggtaggagtagcatgtgacatgtgggccactgcagggaagccccctgctttgccagagaaggatagagaggtaaaagcccactcccttttgttaccctggtaagtggcttttctgaacgcactgtacatgtgctgagagaagcatcctgcccagaaaggcagctttgaggttttttttttaattaagcaaatatttcaagttgtggattctttcagagttattccatcccttgtctgctttcttaaaaaagcttcaaatgaaaatgggaagttataacttgagagacattttgaaaaggagtctgctctcctgccactttgctcttcctagatgaatacgttgccagcttgcatctgccatcatttgatgcccaccttacagagctgacagatgaccaagcaaaatatctgggactcaacaaaaatgggccattcaaacctaattattacaggtaacctggggagaagcaagtgaaaagctctttttcccagtattaaaccaagcaggctagcctgcttctgacataaagatcaaatgttgtacctgattttgaagaatacaagaaatctgttccaggccaggcacggtggctcacgcctgtaatcccagcactttgagaggcctaggcggatggatcacttgaggtcaggagttcaagaccagcctggccaacatggcgaaaccctatctcttagccgggtgtggtggcgggcacctgtcatcccagttactcaggtggctgaggcacgagaatcacttgaacctgggaggcggaggttgcagtgagctgggatcatgccactgcactccagcctgggtgatggagtgagactctgtcttaaaaacaaacaaacaaacaaaaaaactgttccagttaacaaggtagatagagaaattgtgggagatatcagggagttagggtttttttgaaattttggagcctgggcctcaccctaagagattctgatatatttggtctgcgatgggaccaaatttacaaaacattattttgtaaaacctccccagattattgtaatgtttagtcagggttgaaaaaacactgactcattcctgggaacaggcggtaacaccagaaatggcattgtcagagaccctgtggaagaataagggagactggtgccctggggaatgactgcagggatcccacccaggctggggaggggccctggagaaggtgctctgtttccgattgttttttggaattctcatatggaagacataagtgttaaccaatcactctctctctttacagatactaatggaccatactaccaaggaccagtccacctgaaccacacactctaaagaaatattttttaagataacttttattttcttcttactcctttcctcttgatttttttcctataatttcattcttgttttttcatctcattatccaagttctgcagaccacacaggaacttgcttcatggctctttagatgaaatagaagttcagggttcctcactctagtcactaaagaaggattttactctcccagcccagaaaggtgattctttctttaccatttctggggactttagtcttaattaggtaccttattaacaggaaatgctaaggtaccttctctgtggaacaatctgcaatgtctaaatcgccttaaaagagcccatttcttagctgctgaaatcagtgctctttcacttcttcagagaagcagggatggtacctacccggcaggtaggttagatgtgggtggtgcatgttaatttcccttagaagttccaagccctgtttcctgcgtaaaggtggtatgtccagttcagagatgtgtataatgagcatggcttgttaagatcaggaggcccacttggatttatagtatagcccttcctccactcccaccagacttgctcatttttcgagtttttaactagactacactctattgagtttaattttgtcctctaggatttatttctgttgtccaaaaaaaaaaaaaaagaaaagaaaaattaaggagaatttttggtgttaatgctgaggaattgcttgagtggttagttgttaccaatttctcttttgaacctttggagctaaggatgctgagtctagagaaatgctagtctcaagccctgttaagtccctctgtttctagcccgtagttcatagcatcagtgaactggagccacaacagcaaattctatcagctgtgtaccatacagcttgtgctgaaggcgaatttcttgagccattactcagtataaagcactgagttctatctttaggatttatctttaagagcaaatttctggtcagctgtgcttctgcaacctaaaatatttaaagggaggtaggtgtgggcaggaggaggaatgataaattgggccagggcaagaaaaatctagcttcatataatttgtctgggactatacaccctatataatgttagttttacagaagtaatatgacttttgattgctacataccacaaagagtttatgaactgagatcataaagggcaactgatgtgtgaagaaagtagtcagtacatcctggctcatgctctgaaagaatatccagagaggctctctcaaagatcagggagatgtattcccatgccatgcaccctgcttcccagcatttctgcatggtcaagtgagctttatgctcatgagctttaagtatataattatccaggattttaaatcctcaacttgttctagcttgtgatccctcaaagttgggtcatacgttagtgctagatactagaaattttcacttttccactgatcagagagacagacattaaaaacaaaaatagaagaaaggaaagctttcaccctgcagcttcttagcagggaacaattgtcttgccaaaactttgtttccctgtttctgtgcccatgtttgctgtttacccaatcccttcttactgccttgccagtgtgacccatgctttgttgtctgtagatgttaacagttaaggcctattttcctgcgggcacttaaccaaccaatcagaacaccacatgctgttaggggaggtaacctggccaacagtgtatccatcacgttagccctgctggagggaagggacccacattcacctgccctctgacctgccccttgatcccatatctattaccgtgtccataggaataataggtaagggctctgtctctgtcaagccatgtaacaaaggacactgttaaaaaaaaaaaaaagtctggcatcagagggagcatgtggagagcaacttgggaagaacaagttcattttgtattgaatgatttttaatgaatgcaatattaatccttgcagatgagcaataatcattaaaatcgattaaaatgataagaccttattggcagtccgtatgtgtcattctttgttaaacagcaaaacttcattagaacctgttttctagggtgtgacaagttataggtcagtagcatttaatagagatttgttgagggcctaatatatagaggctggatacaggattcttcaaagtaatacctgaagttttgtatgcctgctctgtgccaagcaccgggcctgggtactttcactcctaacttccttcaggaagcagacttgctgcttaagcctcagggttcctcatttgcatagtccttctaagacctaatttctgaagaatgcccccaaagttatgtaagcttcagcctcctcccacacctgtatccatttctgaagtggtctcagtgtcttttttttttttttttctctttatttttttgagacaaggtctcactgtcacccaggttggagtgcagtggcttgatctcagctcactgcaacttctgccttccgggctcaagtgatcctcccacctcaacctcccaagtagctgggaccacagtgtcagctaattgtttgtatttttggtagagatggggtttcgccatgaaatctaggctgatctcaaactcctgggctcaagtgatcctcctgcctctgcctcccaaagtgctgggattactggccagtgtcttctgatgtaagaaagtatttatcttggtcagtttctgctgctccagcaaaatcctgagactgagtaatttataaagaacagaaattgatattctcacagttggaggctgggaagtccaaggtcaagtcgttggcagatttggtgtctgaagagggctgctgcctgcttccaagatggtacctcttatgcatcctcatatagcagaaggtagaagggcaaaaaggacctaactattacctcatatatgccaaagtctgttttagatactggggatacatcagtttaaatcagatgatctcttcctttgtggtgtttacgtaaaattccaacaagatccaaattccttccctttttcctttacccacaatttggccattaggtaagacagagcaagctagggattcatgtggatttgcagcctgtgagtgtgggtggtatccttgtgggaactggggatgtagcttggtacaaggagtcagatcccaagtggcgtggggaatgcatctatgcaggggatgaatggcaatggcagattagttagggataagggatttgatcactgtacgtgaaaatattaagggtagcggaagccagctgtctcattttggaggaaggtgttacacattggaaagggaaaacactagaatgaactctgctgttgggtttgaataggagatgatagtgtgaagtcataaatatcaacctagttaaagaaatacagatgtcaatgtacgtgtgtgtgtgtgtacaaacactccctagctctctctactaagcaagccttggagcagcagttcccctcttcccccagtgcacatacctgacctcgaggtctaggcactacattctccaccgaaagaagccaggactcgttggagagatgactgattccatggcagagcaaaaaaaagtacaagataaaccagaaagcaaggaaggccttaaaaaaggatacataagccagctgtaagggatttacatggggtaaatctgagacaacctgagcatcagcatagtaatgatttgtaatccattgaacaaaatagaaaactatgactctgctaatagaaatgaatgagtaagtaaacagaagtttaatgaggaattgtatatgtatatactttttttctaatttatttatttatttttgagatggagcttcactctgtcgcccaggctggaatgcaatggcatgatcttggctcactgcaaactctgcctcccgggttcaagcgattcctgtgcctcagcctcccgagtagctgggattacaggtttccgccaccacacctggctaatttttgtatttttagtagagatgggctttcaccatgttggccaggctggtcttgagctcctgacctcaggtgatccacctgcctcagcctcccaaagtgctgggattacaggcgtgagccactgtgcctggcctatgtatactttcaaagtagccttccataaagtatttcttaaataaaataatggggaaaagagtagcttcacagtagtgaagtctggctggcagaccaccttaatcaatggaaagaaacgaacttcagtaatgggacaaatctagatcttcccaagatctagacaaatctagataagttgcttgagaagaacacagcatccttctttttttttattacttttttttttttttttgagacagagtctcgctctgtcgcccaggctggagtgcagtggcgcgatctcggctcactgcaagctccgcctcccgggttcacgccattctcctgcctcagcctcccgagtagctgggactacaggcgcccgctaccacgcccggctaattttttgtatttttagtagagacggggtttcaccgtgttagccaggatggtctcgatctcctgacctcgtgatccgcccgcctcggcctcccaaagtgctgggattacaggcgtgagccaccgcgcccggccagcatcccttctatgacaaatccacattgaaggacactttacaaaaggactggcctataatattctaaagtatcaaagtcaaagaatgactgaagaactgcaggagactaatacaacaactaaatgctatcctgaacttgttcattttgctatataaagatacaaaggaccttattgggacagctggtaaaacttgaaagggcttggaggatgagacgatagtaatatctgttaatttccaaatgttaatggctgaactgtggttatagagaatgcttgtttgtaggaaatcaacactaaagtattagaagtgatgggttatcaagtcaacaattcactttcagatgggtccggaaaaaaattatttgtaatatacttgctacttttttgttagtttgattatttcaaagttaaaattttgttcaatgagaatttgaagataaaaaagtcagaccagcttgatcaagaaaattttaaaaatcaattgtgctctatttagggacatcttagaccaaaaataaaggtgaagccttttggattagtaaaggagaataaaaatcaacagaaaaacagtatttcacatgtctatacaaaaaaagtatttcattgacaatacgtcacaactaaagtaatacacagacctgggggttcgacaagaaagtggaggccggtacagtggctttctcctgtaatcccagcatgttgtgaggccagggtgggagggctggttgagctcaggagtttgagaccagcctacacaacattagtgagactcatttaaaaaattagccgggtgtggtgctgcacgcctgtagtcccagctactttgagggctcaggtgggaagattgcttgagcccaggaagtcaaggctgcagtgtaaccctgatcacgccactgcattccagcctgggtggaagagtgagaccctgtctcaaaaaaaaaaaaaaaaaaaaaagtgacgagtggggttgcaagaaactactccagagtgttccttggttatggatactgaacaaagtcttccccaccaaataacttctgaattcaggacatttctagagaaatttgtaaattttgaaagaacgtaaaagaagaattaacgtcggtacttctctgctaactgggtatgaaacactgggttagagcaatggttggcaaaattatagctctcccaccagccaaaccagcctaccgcttgtgtttgtgtagttcacaagctaagaatggtttttacatttttaaacagttggaaaaaaaatcaccaatattgtgtgacatgtgaaaattatataaaattcaaattttagtgtccaaaaatggaacttttatacacagccccactcattgatttatgaaatgtctatggctactttcatgccacagtggtagagttgaataggtgcaaacaaatggcctacaaaacccaaaatgcttactatctggccttttacagaaagtttgctgatcccaggtataaaggttgtgtcaagtatgaagggaaagatgtatcagaatgtgtctgatttgcagtctgttaggtaagaagagggatctctgtgtgagagcagaaacagagaatgaacatattagaaagaaaaggatatcaaagagttttccagtctaggttgcagatgctaaattcacctgcatcctggccaggattcactttaggattaggaaatctcagcaaactgataaagcaactcagatagtgcttgggaaggtaattgttattgcaatttagaatatgtacttgagtaatcaatttaaacttgcaattttaagttgcaaccaattttacatacagtattggaaatttatattatgtattggaaacatataagtacacagtatcagaacttttaagaatagtattttggccaggcatggttgctcatgcctgcaaccccaacagtttaggagaccgaggcaggcggatcacctgaggtcaggagttcgagaccagcctggctaacatggtgaaaccccgattctactaaaaatacaaaaaattagtcgagtgtggcggcatgtgcctgtaatcccagctgcttgggaggctgagtcaggaaaatcgcttgaacccaggaggcggaggttgcagtgagctgagattgcaccattgcactccagcttgggcaacaagagcaaaactctgtctcaaattaaaaaaaaaaaaaagaaaaagaaaaagaaagaaaaaagaatagtattttgtttgcttttattttgtttgtattttgtgccagacaattgaagtaatcccaaaagtgaacttaatatattaaatgtataaacatatttgaaagggtttaaaggagtttaagtttttaagtgggtgattcaaaaccccttagaagtcattaatatttgctgggtttttacaataagatttataagctgaacttttttgtttttctattgagacggagtctctctctgtcatccaggctgagtgcagtgtagatcttggctcactcaacctctgcgatgatgagctggattctatcatca

SEQ ID NO: 2 - protein, 530 aa, human
MSMPDAMPLPGVGEELKQAKEIEDAEKYSFMATVTKAPKKQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPSFDAHLTELTDDQAKYLGLNKNGPFKPNYYRY

SEQ ID NO: 3 - DNA, 2655 bp, Human
  5′UTR (1)..(359)
  CDS (360)..(1949)
  3′UTR (1950)..(2655)
ctttctccccgcctcttccccctcccgggagctgccagtacttgacgtggcgtcaccgccctctaccctcgctttgcgtgcgtgtttgcgtacagcggaggtggcggcgcgggcaggtcggagctcggagctgctgcttctggttctcttgtggccaccgtcgctgtccggctgccttgggctgccgaacagacaaggcgtgggccacagcacctcagaagccgacgcagctcgacgcaggggccggcaggagggtgggcgatcgcgtgtcggagggcgccgcgcgggcaggcgggcgggcgccagagggggaaagaggcgggggcggcgggtcagccgctggccgggccggcgggggaatgtcgatgcctgacgcgatgccgctgcccggggtcggggaggagctgaagcaggccaaggagatcgaggacgccgagaagtactccttcatggccaccgtcaccaaggcgcccaagaagcaaatccagtttgctgatgacatgcaggagttcaccaaattccccaccaaaactggccgaagatctttgtctcgctcgatctcacagtcctccactgacagctacagttcagctgcatcctacacagatagctctgatgatgaggtttctccccgagagaagcagcaaaccaactccaagggcagcagcaatttctgtgtgaagaacatcaagcaggcagaatttggacgccgggagattgagattgcagagcaagacatgtctgctctgatttcactcaggaaacgtgctcagggggagaagcccttggctggtgctaaaatagtgggctgtacacacatcacagcccagacagcggtgttgattgagacactctgtgccctgggggctcagtgccgctggtctgcttgtaacatctactcaactcagaatgaagtagctgcagcactggctgaggctggagttgcagtgttcgcttggaagggcgagtcagaagatgacttctggtggtgtattgaccgctgtgtgaacatggatgggtggcaggccaacatgatcctggatgatgggggagacttaacccactgggtttataagaagtatccaaacgtgtttaagaagatccgaggcattgtggaagagagcgtgactggtgttcacaggctgtatcagctctccaaagctgggaagctctgtgttccggccatgaacgtcaatgattctgttaccaaacagaagtttgataacttgtactgctgccgagaatccattttggatggcctgaagaggaccacagatgtgatgtttggtgggaaacaagtggtggtgtgtggctatggtgaggtaggcaagggctgctgtgctgctctcaaagctcttggagcaattgtctacattaccgaaatcgaccccatctgtgctctgcaggcctgcatggatgggttcagggtggtaaagctaaatgaagtcatccggcaagtcgatgtcgtaataacttgcacaggaaataagaatgtagtgacacgggagcacttggatcgcatgaaaaacagttgtatcgtatgcaatatgggccactccaacacagaaatcgatgtgaccagcctccgcactccggagctgacgtgggagcgagtacgttctcaggtggaccatgtcatctggccagatggcaaacgagttgtcctcctggcagagggtcgtctactcaatttgagctgctccacagttcccacctttgttctgtccatcacagccacaacacaggctttggcactgatagaactctataatgcacccgaggggcgatacaagcaggatgtgtacttgcttcctaagaaaatggatgaatacgttgccagcttgcatctgccatcatttgatgcccaccttacagagctgacagatgaccaagcaaaatatctgggactcaacaaaaatgggccattcaaacctaattattacagatactaatggaccatactaccaaggaccagtccacctgaaccacacactctaaagaaatattttttaagataacttttattttcttcttactcctttcctcttgatttttttcctataatttcattcttgttttttcatctcattatccaagttctgcagaccacacaggaacttgcttcatggctctttagatgaaatagaagttcagggtccctcactctagtcactaaagaaggattttactcccccagcccagaaaggtgattcttctctttaccatttctggggactttagtcttaattaggtaccttattaacaggaaatgctaaggtaccttctctgtggaacaatctgcaatgtctaaatcgccttaaaagagcccatttcttagctgctgaaatcagtgctctttcacttcttcagagaagcagggatggtacctacccggcaggtaggttagatgtgggtggtgcatgttaatttcccttagaagttccaagccctgtttcctgcgtaaaggtggtatgtccagttcagagatgtgtataatgagcatggcttgttaagatcaggaggcccacttggatttatagtatagcccttcctccactcccaccagacttgctcatttttcgagtttttaactagactacactctatttgagtttaattttgtcctctaggatttatttctgttgtccaaaaaaa

SEQ ID NO: 4 - protein, 530 aa, Human; identical to SEQ ID NO: 2

SEQ ID NO: 5 - DNA, 33000 bp, Mouse
  mRNA (777)..(1133): Exon 1
actgcctagagagcatggagcaggagaacccatttggctgggactttttctcaatctgccctgttctctgccctctgaccaacccatcaaccttatcttaatgacctcttcacatcagcactgcagcctgaaggtcttgtaaacagacaaggcccaggaggagatggtgatggaggtcataaagggttgtgtagaaaccagactgctctcagcttgttctgcacccctgtgaagtaaaaaagcaaacactgtcccctggaaatggacttctggcccaaagggcccagggtgtgggccgggagtaggctgtctctaacactgaattttcccaaaaagggccgggtaaaatgtgtccggtgggactctggtttatgctcccaagcatcgctcttgatatgccatcttcccgatccccgatgctctgccacctccccatgctgaggtttcctggggagttggtagtccaaagacccttagtcttgtggcatctttataggagtcacactcggctcaatagggctgatggcaccacctggcggcagacaaggttcaactactcaccctgggacgttcatcaccgaggcgcaggaaaggaggggcgattcctgcggcccggcccagcatgttgacgtcaccagagcgcgccatttgtccccgcctctttccgcccccctctccttggggctgccagtacttgacgtggcgtctccgccctctacccttactttgcgtgcgtgcttgcgtgtggcggaggtggctgcgcgggcaggtcggagctgcgtgctgttgcttctggttctgtggccgccgtcgctgtccggcaggctccggtctcggagccgacgaggcgcgcgagcgcgggtcccagcccttcggaagcccaagcagctcggcgcggggcctggcgggaaggcgggcgagcgcgtggccgagggcgccgcgcggacgggcgggcgcccgtgagggaaagaggcgggggcggcgggttagccgcgggccgggccggccgggggatgtcgatgcctgacgcgatgccgctgcccggtgtcggggaggagctgaaacaggccaaggagatcgaggacgccgagaagtactccttcatggccacggtcaccaaggctcccaagaaggtgcggggctctgggtggcggcgagggccctcgaggaaggggcgcgagcaagccccgggcccggactcgggtagttcgggacttagggctagggtttggggtgctcaggaccattgacacgatgtagtgggtggccggtgagcggcgatgggcctagctggagccaaagaggccgctctgacctcgcttttctttgtccctcggccggagtcctcctggatcttactgaagcccgacagggccaggccttaaattcgcgactgacttttcctggatggagggccttgaagacgagattgggagggggtgagcgactcagctgacccctgtgccctgtccaggggatgaggttgtgcaggaaatttcccctcaggtgagtgggggaaatcaggcagctctttttcaaagcatcttcttgggggagaattggccgttcgaccaacaactgcgtgatcgcttagaggcaagaggaaagaatcgtatgaactggtttatgcagctgacggttctaaaagcaacaagagatggaaaggcaaaatggtaatcgccgtgcagtcttccacctgctaggaagtatgtgagaaaggaaaatttagaggttggtgacagtagacactgtcatggatgcaggttgatcgcagtcatgtgaatagatcacttagacatccctccagaaaggagcttgggtgggttggggaggtgggtgggaagaaatgattgctcattctttactgcacttacaagttcagcctattgtgctgggggttgagctcaaaggacatccttctatggctcttggaaaaggtgatctatggagggctgagtctaactagctctctcagtggtagggtcggggtctccagtgctttctgtcgaacctttgctgacccttcctctaacctggtacccaattagcagtggaacaaggaggtgacctttctgaaatagtttcctcttgtataaagtctcttcacaatcaatcctgcctacagtatttgtagggttcaaggatgatatagattcagctgagcaaggtttgcagagcagttaatccccaaggctcctgtactggaactagttaagctgctctcagtgcctgagagggctttatttattttcctccaggaaactaattgactggtcttggggaaaaaaaaaaaagtcattgctgtgtgctttagctctgttttctcacaaaattgagaatgtcaccaataacacctttgaaaggtttatagaaggtccctccatcctaagggatccagttgacctcatttttagtgctctcattccccaactgctttgttagagaagtgaccttttgcaatctgcactatttgaatacaaagttagagttcttacaagcaaaatgcatacgtgcagtacctcagatgagttaagtggagaattggaaacactgatagactaaccccctgactcttatctcttgtcataagcccaccttcttttgtctaggtactccagagagtcttagtcatttttattgttccttatttttttctttcagtgtaactatattcttgcaagtcaatattgataagccaaaatctgtattatacattgtaaactcctggaggccaggactcttggttaccattgtatctatggagcctagttatggagcctagttttgtcccttgaatgtagtactcaattattaattggcaatctggctaaccagtaactgagatgctgaatgccaacagcagtaaatatcccttttcctctgtctgaaaggaatatcacaaatgagtattattgtgaagccaagtgctaatgactttagagactcatattccattaactaaaataagcaagtcctgccaggcgtgtgggaagaccctgttgtaaatttccatttgctaatgtatgtgtcctcatgttattcatcttgttctttgagctttattgtgtggtatgtacgttgtacttgtattaagatgtattatgatgactaattaccttcccttaatgatgatgataagaagcatttacttagttaatggaccagttaaggactagtcaaggtcatgtagtaaattctgggtactctggttttggttttttattccctctcaggatttcagttcctcgatattctgacttaggtcactgttgagcctcttccatgtggtgataatgtgatgaagtatagaaaaggtgagggtgcaggtggcatggtactgaatagtgtgaggaaaaggtctctttaaaatgatgtagtccgtcatcccaaaggatcccagctcgactgtagaccaggtaatctttggatcattcccattactgagctctcgctggaattgagggcattcctggggccataggtgactttaaagcttctccatcaagtcagcttctagtatgcttgtcttgccagtttttaattattgtttgctttgtttgaggtacagtatattatatgcccattcaagcaaaagcatatcttattctctaactaaaatgatagccagttttatattggatgtctactccagtactatatattacagggcttgttggtttgatttaagacttgaatatttggcagtcagaaagccaactattaattggtatccccctaagtgaataactcttgtgcctcagtttctgcatttgcctaataaatgtctgttgtttggaacaggtgatgccagagatatttggaacttaagatagtaacagtgaattttttaaagtgatattttactccctccttaaaatattttcatatattaatttatggagggaggacatgtgtgcacagaggagtcagtataatttccatgagtctaatccgatcaaactcaggttgtcaggcttggcagcaaatgtctttacctgctgctgagcatcttgctggccctgtcccctcttttcagtgagcatttgttgtgacttgtagagagtcataaatgccaactactgagaagataaaacttaagaaataatgggaaagtattgttaactttgaaggctaagataattagtgatacagattatcataagcaactctgtaaaacatcctaacatccagaagaacatgaaaagaaccatagtcttgtctcttagtagacatgagaaataatggaagacattttttcctgacctagaattataaaggcagttttcatgggtccatattgattgctgtggtatatcctaaccccacccccaccccccggtttttttgagacagggtttctctgtgtagccctagctgtcctggaactcactctgtagaccaggctggtctccaacttagaaatccacctgcctctgcctcccaagtgctgggatcaaaggcgtgcgccaccactgcctggctgtggtataacttacatgataaagcttattcacaacttataagtggtttcagttaggatggtcaggaaaggttgaagtgagggctcttttagtagtaaggagaagtgtacattgtaccacaactgaaggagaaccctgtttaatgttggcagttactcaagacaaatccaatacattttgtggtgtagatagtaactgtctttggaacttcagaaattgacattgtgtcagattgttggttttccataaataaaccccaatctctaatttattgatgaaaactcctttgatggacatgattctctaaatacctctcagaatattacccttctgtaaatttctgacatctaaggaggaaggactcaggatcctgctgagtagcatgttaaacgtttagcctaggttctgtgcagttcttttactgctttgttaccatttaaccaaattgttaatgttatcttgttgtagaagtttattttgaatttagagaatgtaaaccctcatcagtgtttctaagtctagaaaaggtgactacagagtatgtgtagaaggcagagaattagaattggtcttactttggcctgaggaaatgcctttggtagttgctgcattagagactgtgcataatgcttcttaatcttttgagccttaatctttgaagaatttcatgaaatcatagtttctgtccttaggaaaaatcagaagagcacaaaaatacatatttacttgtataggctttgatagggcagcctggaactcattcatagaccctaacccttgctgtaatttgacaggcttctaagcatagtatctcttttctttatagagctagattctgctttaaaagaaaatgtttctagcttgcactttgtctttgaggtttggaataaagctggggctggtggtattgttaacctcctctttgtggctcgttattgccttgagagtataactcagaaacttatacacagaaaccagactttgagcaactgtgggtaagtactttctcagctctatttctctccataggaaactgtatgtcttaaactattcttgatactgaactgataaagtatggattttatcatttgatttctctaggtagagcttattgatgtcgaggactgtttcagacatataaggtctgacacacagtactgttgatgaacgtttattgagtcactactgtgaagtgattgtccccaggagaaagtaaggtccagtcacgtagtgactgacttgctttcctttcttattgccagagctgtttttatggaaagataaggcagtagctagggactgcaggagagaacataatgagagtttagtagctctctttctaatacaggaaatggaagggaagggaagagaaagaatacatactacatggcctcctcattagctgtgtagtatggagacataaagcagtcagctcagagctatccttactgtaatgagggatgcagtttgaaattctcaccgttggagcaaacattttaagcactggggaccctctctctgctcaacgttgaaactggtatcttagatttaagaaactttgtccccaggttgttgcaagtttgaggaataatgaaaagttgggcagcaggatttggaatgtatgttagggctctgacagtgttaacagccttgttagcaatgctgagccgtttcccatctttcatactgtgtgatgctgggtgtccattcagccgaaccaaaagctgaacttgacatccaacagtctgatgtgcacttgtccttgaaacacacctttacagttacagtcttctgggaatgagccagtgctgtcctaaccatgagctcctgttctcgttggcactaaactttgtgatgtttatgtaaaatgtgtgactttggcaagatttgggggaaagggagtctttcaaaattccctgttttggaaagtgaaataagtgctttatagaaatggcagatacatgcagttactctggtaacagattcagtttcctgtccgtgtccaggtggttccaacttctaattcagtacttgagtttttagtgccagcatcagtaccagacaggaagattcccagtgttggcttgttatgtagagagtgacccagctggcttgttgtgctggacttgatgaattgtgaattttaattacactgatcaatacagtggccttggtaacggccttgtagctgctgtagaatctgtttgcttcagggttataacagaaagagactaagttgatcggggacatcatgttctcactgtaataagctgtaggctaagtccaagaaaaaacaaatcctggaggggctgacctcactacaccccttctccagtatttcccaggaggaagcattactctggaggtggtttctgttcttatttgccagctgggagtaaatcaaggttgacaaagacttggttcttagtgtaagtggggtaaaggactcttccaggttctaaagggcacatagaaaacacttgtactgagaaggcccctgtctgctggtctgtaattgggaataggtgattgatgaaactaaccttagccctctcattcaggaattagagctttcctatttggggaatatgagattcataattatgcacattcttttctgattccatagggaggggattagctgaaagctgggcatgattggaatgagagcatattgccagtttttgctaaatttgaactaaaccatgtttaaaaataaataaacaaaaaccattctgtcttgtttagatagactctcatgttctcctaaccctttatggatccatcacatgcatgcctgtactaacaagtatagttggtattaaaagtagcttctccacttcctttcccttgttttaattcggagcaaagcttattaaacccatttatttcctgcatttttttgaggtgctttgtcaaaagcatggcaaactcagtgtaattgtttgctttttcaggtcctccttagattgcttgttaaatcagtacctttcgtgaggttggagtgtgaaagatatttgtcacaaagggagttttttcagactatttattattagggctctaaattagacatacttaattcacagtaggagaggacctgaaggtagggcagctagtggagaatgggttggcattttgaatgaccaaggcgctggaaattattttgaaattagatatggggtaggagtgcagagagagagagaaagaaagagagagagagagagagagagagagagagagagagagaacacacagtaaataaaacaatgtaaatcaaaggaattaacctgaagcctgtcattggacctaagctaatgattgcttatctagaattaaggatatgttagggccatagaaagccctgagctatattctttggactttatctcctaaacagtctttatcaaaataggaaggccatttatgtaagagtactggagggtatctatagaacaaatggggaacaccaggcctctctaagccagtttcatttcttgagtctaaattctttttgaaatgctaagtataattctaacacattatacatacccttacctctagtttctggatggtcaagtcagaatatgagagcttaaacttgttagtttatattataaactcttagactgaaaataatagtattttaaagactttgagaaacattttggtaaaattctgacctcagaagaaaagcacagtttagtatagttaattattgaagagttacaattcaactaaagaatagatcagaaacacatgctagggaggaaaagacagaacttagaaagttgatgaagccgatgagccatgttaccagctaaagccagtgttaatgtggctctggtgtttgatgtgaaagaatggccaggcctccctcaggatgagtgctgcaggagcaaggctgaaactccttcggggtactgaaaaagaactcagtaagtggatggttcagttgaggctggattctaagctgaccttaggatcaccacagcccaaatgttcctttacttgaaggaagttcttagagaattattatttaaggttttgagaactttccatgcccatactgaaagactgccaaataaggagtattataagaatgttccaagctaactttgtatagagactcatgtcccattaacagaacctcatctctccccagaaaagtcaggagactctacctggttccagttctaactgctttcttgtgggaaaacaggaagcctgctgttagcataaggatatttcatggtttgcttagtgatacattaaaactgagaggatatgatcatgtaccagttatctttacttactggctggagtggtggctcagcagttataagcattcattccctgctttttcagaggactggagtctcattcctatcgcctaagtcaggcagcttacaatgccagtaactccagctccaagggatccaaggccaacatctgttcatacactgaaaaataaaaataagcataaatatttttaaatgaccaaagaactttaaacctcacttcctggggcttttaaaatctattttacagttttattcaacctgtaacctaatactccagtttttatttttcttcattaatttttttcagctttttaaagatataatgggaaaaaattaaatgtacttaaagggtacaatgttatactgtgttatttgtacacagtcaagaaatgactatgatcaagccagttaacataaccatttcctcagttctcttttgtgagaagttcagcacagtgctagccattttgttgctgaaagcagctccccagaaagtactccccaaaaggttcagttttcaacctgtgggtccaacaacccttttataggggtcgcccaagaccttcctgcatatcagatatttacattaagattcataatagtagcaaaattacagttatgacgtagcaatgaaaataattttatgattgggtcaccacagcatgaggaaatttattaaagtatcttgagtggactggcccttgaaacagtaggggactgctttccccacctagttccaaaaggagaaatgctactaagcttgtggcttgtttgggatttttgtttagtttttgttcttttacacttcagtgtcaaagttgccttaagcaaatcacgtaaccccagctgctaccccatcctgtttctgtatgcagcccttagataatgctagtttttgaaaataacaaattgttttgtaaaccataagctattaatttgagacagacaatataagtataaaagtcaaggaaatctgggtatttagaatcttattgcttattaaattttatttctttgtatgtatgcgggtaggtgtgcatgtagaagtctaccatatttgtcccaggattgaattttgctctgtcagacttggtagcattcaccattttccactgagccatttcaccagcctgatattaatgattttattgtttaatatgctagtacatgttgcttttaaacttctttcctcatcaactttaaataaaaattctaatgtagtagtccctatgttgtgaggattgttctttgtgtatcaggagttttttaaaaattgaaaattgtttaaattacaaatatcatggtagtattacatgttgagaaaatagctcttgcaaaacaaaatttaatgtttattttggtatgctgtgcaatattagcaagagaacttgatctttgtgctcctgcacacaccactggctagaaaacaatagaaggcagggctcaggaaaacccaaacttgcatatgctggtccccactgtagatgggtggcatattacactcctggaatgataggtaacttttgttttaaaacaactccagagaagccacaactgacagtccttcccggctttcttgcttcttacaagccacataaaatagtagtttcataataagttttttatgtaagcttgaagtatgaaaactaaaatcagctgaagctgatcagtaggggttttactttctcttttcaagttgaaaccatgtaacccagctataggatacattgtgaaaggtcccattggctcctttagaaataattgtggttgctacatagttactagttaacatgggctcctggaagtcaggtttattttgttatgttttgacagcttgagcctatacgcttatgggaagcagctaatggacaacaacttaaaaaaaagtgatataggtcctgtctggttagctcagggctcaaaacaccagttctcattgactttatagccacacattggtagctgcttatagttccaccacttaaacactagttggtagactatttcttcatctccagttagacatagtctagccttcccaaatgctgagctgactcacagcgtgattcacttactagacatatgcatgaatttaaaatgtaattgatttttaaatctcacataagctgtacttcagaccaaagaagaaaaatttgattttaaatatcatcttcctgttgttaggttacccagtggtgccagagaaccaagtgggcatctttttagatctatccactgtttgtgtttgcttccttgttaaattattttcccgtagtcatgtgtgtgaagtgtacaaataggcctgtttgtaagcaattgacaaggctccctctttaaaaaaagagcatctccacctaggttgcagtaggacatgccaatttaacataaaggtgccatttagcatgtcattggcatagagagaaaggtgaatacagtaagctagcttggtctttctcaggctagcctggaatttatgatcctcctgccttggctaggatcacaggtttgtgctgccataactgtcttctgacctgatttttaatacatgctctatgagtctaaacaaagtgacttccaaagagggacagaatgtgcagaagattttagcatgataccttttgaggaagtaagagaaggaaaatgagaatgtaacatgtatttgctggaagaaagccaacacgaaaacatatggaagccaatgaaaattggctgaagggaagtgagcctctggatacaagtgtaaagctttagtcatgtctgttttatatgtcaaggtaagatttaataggcgagggaaaacatgaaatgttaaaaatctttggaaacacaaatgagctcaactagcacaggaaatctaagctaatgtatcacattggttgtgcaggcttccaggaaacactacttggactgtatatccttcctgaaatgtgtaaggacagtaaggacttgaaagaaatctccttacctttgcatttacgtttgtatacacggtggcacagaatgtggaggtcacaggacagacatgggtgtcaggccttgccttcctccttgcttgagacagtgttattcactgcttcatgtgcctgtgagacttttctgccccccacccccaactcacataaaagcaaaaggacaaaaacaaacagtatcaacaaaagccagctttctaatgtggttctctgatacctgttgtgccctctggcatacgcatgttccgcctgcatatgtacatcattcacaatagtaaaataaaatgtttttaaatatgcacgtttaccattcttttgggttttttgagacagagtctcactatataggtctggctcttctggaactcattatgtagaccaagcttgttcccaaacccacagagagtcatctgcctcccaagtgctgggattaaagacatgcacctctacccagcttattagtcttaaatgttataatgctattgagatttggtaggagagtatcttaattcttaattctcagatgaattccaaagaatttattagaatttctatatttgtaccttattatttgcctctattatatgaaagtatgcatccacatatatatgtgagtgtatgtatataccacatgaaaattcaaaggttcattctaaatcaaaagtatatacatatttattatactgttcttttgaagttttcttttgtgttaagaatgataagtctggggctggggagatggctcaatgggtaagagctctgactgctcttctgaaggtcccgagttcaaatcccagcaaccacatggtggctcacaaccacccgtaatgagatctgatgccctcttctggtatatctgaagacaactacagtgtacttataataaatcttaaaaaaaaaaaaaaagaatgataagtctgagccgggtgtggtggcgcacgcctttaatcccagcacttgggaggcagaggcaggcgaatttctgagttcaaagccggctggactacagagtgagttccaggacagccagggctatacagagaaactgtcttgaaaaaaacaaaacaacaacaaaaagaatgttgagtctaaggaaatgaaagctccagcagccgtgctgggagagaagggtggcaagtccttcctaacaccttactaaagtaatgcagatgctttgtggaaccagagtcgacagttactagtgctgagatgccaggagatgcatgttaggaggggtaggctagcatgatctgaggggaaggcagttaggctaagtagaaagtgtaaagttggtctgactgtgctttgcagtatgttgccaattgtggtttttagaagattagtgaggccacctctgtgctgcttcaacaccttcaaaaaaacagaataaaaaaagccatttgctatttttacctttggttggcaactgcttggttgagactgtagaagccagtgaagagttgtctcttgaagaaaaggggtagtggctgatggtaataccacttgtgctttgggaagttttcaagtattgtctttcatagtttcagtgactcagtatatgggcctgattgcacctcattctttctcacactaacctgccttgacactgtatcacattgattctcattttgaaaggcataatttgtctgactgtggaattggattctagtagtgtgttattggtgacatccaaaagggtagagtggaggaaagatatgtgtgttgtgattagtcagcctgcccacccactccctcctggaaactcaggccccatctggggagactagaacttaagaaacttgagacagagggaagatggaatagccaaaacctgctgggttttagaaaatgtttctgtaccaggaaatgaaagaggctgaagggcctactttggctctgtagctcaaaaatgatcctttttcaattcgtcttctctgatgtatgtgtttgagggcaagttattggaaagaaaagaccctcccttttttatttatctatttattttatgtatttgagtgttatgttttcatgaacaccacaaaacagaatcagattccattatagatggttgtgagtcaccatatgggtgctaggaattgaactcaggaattgagccatctctccattcctcctttaggaaactatttttatctaactgaagtaaaagatagtagacttcggtttttcttgtttgacagtattgctatttattggtggtgattttggaggggaggggatacatttaaaataggaatcagactgatctctgctaagacggaaaaaaaagatgctatattggggagggtagctgactgtttgacctcttttctatcctctgaaaggtagtggctggaagtagtcttaaaaggaataattagctggaagatgtatggagaagggatttttaaaagatgtaggaaccttagacagtgttttaatatacatcttagtaattgtcgcttgttaaattttctaggttctgatagtcttttttggctcaatgctggtacctaagaaatatgctacccataatttcttaaacagcagaattctgagcacaatatgcacagaaggcaatgggacagaatatgtatatttttaaatgcctcagaaaaccaaaagaggggtactggggccctcagttaatctcagaggttagaaagacgggtcttcccatgcacacctaaggtttgctttcttaatgatttgttttcctagtgcttgctgtttacctttgtgtctagttcttcacccttaggagaaaaccttcctgaggaaattaaaagctgttagataggtatgtgaaataacaaagttaataagatgcctggcctaacctctaatttttggaggacccacattaaacgatatggcatagacgcaagacatcgtgtctactaaactatttgaaaagctgctttaaaatagcatcttaaactggcctgaaatttaattgggttttaaagatacaacaaatagacttcagtgtttgtgtgtgttatggtagccagcgtgttcctagaggaatggtgaaagttcacagcaaagccatctgttactattacatggagagttgaagtacatacacagattttgagagagctttagttcttgcatcgatctttcataggatgaagtacttagtgctaagtgaatatgttgactaaaagaatataatgcacgttgtatattgtcctgatcattgtcacttatccataggatcatagtagtgttctagtaggttttcattttaatttcctctaattggatagagagaaggtaaatttgaattaggtatgggaagaggggagggcagctgagctatagtgtgtgtagatcatatattttcagaagtaaactcaactttgggtagtaaaaagtattaagatcaattaaacaaataattacagttatttttcttttgaagtttataacgtttttgggttgcctgtgttccctttgtcttggtaagtactgtaccatcttgtagatcactctgtcactataccaaccataatgaacccaaattattaggtcaagcttttcctaatgggcctcagtgttacagagaactcagtccaaccatcataagttgtgctttaaaatagcacactatactgatatgtactatactggccttcaaatgcagcaagtagacattgtttggatgtgctcgatggaaaatggtcgatgtaccctatccttcctcagatatgctgttaatctgagagtggagccgagttgtacttggggtactttgtctatgtttccttctttcttccttgtacttactaccttatgctaaagcaccccagtgaaaacctaacagttggaggaaatcataacaataagaagtaatacaaaccatgcttttgcaattgtctttgggttttcttttatccaaatgaccatatttttccacttaactactgagtttaacccaaacagaaagctgctattagatctggtttcactagaaaccagaagttggttttggcagcagtatccttatttacagcataaaggtgaacagcatgcgaagattctttccaccaagggaggaacagctgctagctggaaagaacctgtagagttagacccctgggggaaacgggcagagccatggcattcagaagcagctgctgtgagacgagactctgcagcttgtagttcagggtagcagagaagacaaggatatcatgcatctgtgtgctgctggaccaccttacagacagctatccactatgccagtacttgagttaacactgcctacttcaaatgtggacaagatctattatttgagagatggggtgttaggagcactgcacatagtagtaagctacttgccatagtctactatttttagatgggaaagattttgctaagctttagaccgctgataagctttattaatacaaggagagattatgtctattaatctccggttgctgaagaaatatttctaacttgctacaactgcttctctatgttttttatctaaaaatctgcaagtgttactgaagtgccttcccccacctcactgcaacgtaggtggtatctggaatcatgcccagaaccttttgaaaaaaaaagtagaagaagaaggggaggaggaggagagggaatctaacagccaggagaagggagcacttccgaacaggctgcccatgccagcagcagggaaccctgtggaggaagtgctgcctctggagctgttacagccactaaggaactcttgactatatttttttcactgccaaagtaactgcatattaggttggaattcgttgctttggtcatctctccttgatttacatcttgcctattgaagaagtgaaagtgtaattggaaatcctatttagaaggatttagtttttgtttgtttttctttttgaatgcagctatgcatttgaagtacttaaaattaaatttttaacatttaaagtaaaatatcttgggctttatgatgatatttgttaatcatattctgaaatgatatatgtggctcagcttgggttgtaattgtctttgaagcttgacttaatattgtacgagaaaagagttttaaaggtttacttattctgcagatgtttattaaataccttcctgcatgtgtgaggccctgacctgctctgggggtatgtagttcttctttatgtcttcagtgtctgttttcagacttaataaaacaggctctccctttctgtgtcagagttgcctgaagaagaacttccacagttagatggtgaaccgagatgagttgtaaacacgcaggtttctgtctctgatctatttgattctgtcctctgcgtttgtgcttagtgggaaccatgctgaataggaagttcaggacaagccagtgtccccttgtttgctactttatgactgtagagtcctaccagttctgtttttcatttgttcattctaactgccgattgattccagagctacaaaccaaagcttggttcttaggctgcagtgtcaccccagcagagactgtatgtgttgggagaggaggattgtacagctgtagctaatgtagagtctactttttaaaatgtgtcattttgtgtatatggatgatttgcctgtctgcatgtttgtgcacagcatgagttcaataccttaagagaagggagtcgggtcatcttaggctcaagttatagacagttgtgagctgccatattggggctggggatagaactcaggtcctctgggaaagcatccagtgcttttaaccactgaactattaccccagtttctggtttcctcttttctagcttgaaagtgttgatgactgggtcatttgtattatgcagaaaaacttttcacctatgatagtatacagtttcattgatactatttgctaacaacataatttaatagtttctccataggaaccttttcagagctctgagtctgaatccaggttctcaatttatactctttttcaggaatgtgggttttatttcaaattttgtagaaagaggtcatcagaattcatgtgagagttcagtgattctcaacctgtgcattgagacccatttgccaaacttctgtctccaaaattacttacataacggtataattttataattttatgcttgggggtcaccacaaatgaggaactatattatagggtctaaacagtaggaaggctcagaaccactggtttaagccatataattccatatttttacatattactttgtttataaataatactgattacaaactattttcaatttattaaagccagtggtccaaatattctaccatgtagatatggtgacacatacatatagccctaacactcaagttctaatgggctgatacaggagaattgtgagttctgagctagcctaggtcttttttttttttttttaagatttatttatttattatatgtaagtacactgtagctgtcctcagatactccagaagagggcatcagatttcgttacggatggttgtgagccaccatgtggttgctgggatttgaactccggactttcagaagagcagtcagtgctcttaaccactgagccatctcaccagcccctagcctaagtcttttaaaagatcttgtcattaaaaaaatatatattctgcaaatataaaactgcaaataactagttctacctaaaaaataatttattctacctacaaagacctttaaaacttgtcaaattttcttcttttagattcatcctgattagttaggatgtaattaatggagaatttggtgttcgttttaaaacttctctttcatccgtacactctgatttttaaattttttactggtgctacagaactctagggatactacattggttttatgaattagagacgacatagctggctgtaatgtgtttagcgattactagacataccattcaaaagcaaagccatggccagattcagaggaagaatatgctacaggagaaccagatgtgagattatatctgctttctgatagaattgggtgaaaggataaagagtttagtgttagtgtgtaaaagttagataaaagggaagggagaaacttaatgagatgaagaaaatttagtaaaattggatagttactaaaataaatggcttttcagaattgtgttctacctccttctctcccccccacccccagcctagatcttcctaaaataactggaaagagacagtagggttggttttttttttttttaaagatttattttatttatatgagtacactgtctctgtcttcagacataccagaaggacatatcagattccattatagatggttgtgagctaccatgtggttactgggaattgaactcaggacctctgggagatcagtgagtgctcttaactgctgagctagcccaagacaatagttgtaacagagttcagaaagcagcctcttccccgcccttcagatgagagttactgccacagtaagaaccacatggtgacgccgaattttaggaaagttagtgtatggatttggaaaaggaaaagttttcgtacagcaagatggttccaagggtgaagatgcctgcctccaaggctgacaactaagttctatctccaggactcacgtggtgaagagagagcagactcccacagtttgccctctgacttcttgtgtgcctcgggtacctgcacatgataaataagatccatacaattcccgtgtactaagttttcagcttcttaccctttctcttgtagcaaatccagtttgctgatgacatgcaagagttcaccaaattccctactaagactggccggagatctttgtctcgttccatctcacaatcctccacagacagctacagttcaggtaggtatgactttgatgtcccctctgtcccgtcttattcacgggtgctcttcactaatacttagaatgtgagaatcattcccagcaagacttgcagctgaggatgcttttgttaagatgttctttcccaggctgttttctgtgtggtctgtgatcattgcctagttagtggcactgggaagttcctatatccacatttgcaagggggggagaatcagtggtatttttgctttcagaactggcatgtaaaaagaaatttacttttaaacataagttatttgcataaaacatacccttccacccatgtttttctttttgttgctaaaacagcttgaattatgacaataaaatttatatttttgtctccttttatttataagattaccctttatgttaatatgcctttccctgttaatttctggaatgggaagatataaactattgtaatgtgactccagctgtttccctaccctcctgtcttagccatctgtagtaagtcaagtctttgttctggttaaattgtgcctatctcagccacagagaaggtattaagaaataactagaaaggtgtttagaggctgagatgactcagtaaagtacttactctgcaggcataaggacctgagttcgagccacttaaaaagctgggtgggcaaccaaagaatacacatggagggactcatggcactggccgcatatgtggcagaggatggccttgttggacatcagtgggaggagaggcccttgggcctgagggtgttcaatgccccagtgtaggggagtgccagggtgggaagacaggagtggatgggtgggtagggggggatgggatagggtgtttccgaaggggagacctggaaaaacatttgaaatgtaaataaagaagatagccaattaaataataatgataaaaagaaaaataaaagctaggtggggagggggatacattcttgcaatccccagtactggtaaaacaagcagatttctagattaactggctgaccagctgcctgtcctagcctaatcagtgagctccgggtctggggatgacctgtggcacctgagatgaactgaggttggcctctctgactttcctgtgtgcagacacagtcacagaagcttatgagtcttagaaagggaactgaggaaaacatttagagaggactgagacaacgtcatcattcaacagaagagatagttaaagaggaaaggctttaatgttaatgaggattggtatgacaagcccttcactggttcttaccagtagtatttgcattgttttattaaatttcaagggctggttgtgaggtttaaggagtgtaaaaaaaattatcaaagaaaattgtatctcttctctgtgacactgataatccaaccaataaaggcaggataggcaaacaagaacaaatgaaggtaagcagggtaaactgaaatacacggttatatagattctgtggagctgaagcatgcaagaaacaatcccaaataggatggctaacaaaaatgaaaaggtatctgaagcgacaaactaaattagagtggggtgagcagagcacttagagtaaacagagaggggactttcatgtgggaagattgagctttggaaggagaacaggtggaggaagtgaagttctcaaaggtcttcatggtcatcttacagacacccatgagaacgtgtaaagcctgcactggagctgacttgttagagtggagaagatgctctttcctagtgtgtgttggctgtacttctgtcctttgggaggacagtcaataagagagccttagtgcagtgaccagggacatgtccagttcagagaggaaagaagagactggaggggtaatgcatgactgtagagcagactgcatattcatcggaagaatggatttaaattttacgtgctttttatctcagtttatcttcttgacagattttcccccatagctgtgagaaagatctggttgttttctgttcttttctgtattccttgagcacaacctggtataaaatcccttagggattgcttttacctgtatttattttacttataatagtggttctcagaagcagtacttgggaacctgttggaaatgccaccttgaatacctcccaagacctgccaggtgctaactttgtccaggaacctagcaatctgtacttcaacaagcagatcattctgatagatgtgtaagctgagaaccagtggtctatagaaatggcatccttatcactgttactccattcttatagagtctgggtacagtccagaggagccaagtaactgtaatggagcatcaccacaagtggtacaggattaggctccatacttctctgtcacacacactgctggcaaagtatcctagggtgagtgagtgaaagagctgcagtgctaagcatacagtttcattcacaccagctcttagagcctggaggaacagaactgatgacgtaaaagggacctgcaattaaaagtgttcgcttcacttaaataacagtcccctggggactgtactcagtggagtttgggccattgaggtttttctttccctgtttctttgctgtctctagctgcatcctatacagatagctctgatgatgaggtttcccctcgagagaagcagcaaaccaactcgaagggcagcagcaatttctgtgtgaagaacatcaagcaggcagagtttggacgccgggagattgagattgcagagcaaggtaaaggaggaaatgagcagagggccagaagcacctttgcactgcttatcaggacctggacaatttaagtgtcctggaaaagagtttctcacactttggataaacacttctctctatatggaaactgagccttaggattcttgggtgtgttttgttttgttttcttttttttctgttcccagtgtatggtaattacagagtggtttctgttctcctcatcatgacaccatactcacaggtctacagtgcatagtcagcaagtgttggatcataacatagtggttatttgtgtgggatatttttctagctgccaatacagagacccggatttcccagaagcaggtgatatgataggtttctcacctaaggttgtggtagcctggggtctcattaagtcctagactccctcagctggaacggaatacaggtgactgacagtcaactgcatcttaatttctctcttcctctacacttctccaagggtctctgctcttctgtgttttgggttttttcatgttgaagttgtgtttcttactccgatcttgacaagctcaggtaagataatagagaaaaatagaaatgtcttccactgagttcacagtcatccctcctggactgatgaagacatatggaagtctgtttattttggccagtttggaaagtttaattctttctctgctcaggcctttgtatgtataaagcagaagcaagacccgccaattgtacttacatcataacctccagttactccattggtgaaacatctagtcatgagggaaatatgtttgatattttctaccccatttcgccatgggtcagcagacctaacactgctcgtggctggacaggagcgaatggtgttggtagtgtggacctggctcagatcctctgttcttccacagacatgtctgctctgatttcactcaggaaacgtgctcagggagagaagcctttggctggtgctaaaatagtgggctgtacgcacatcacggcccagacagcggtgagtttgttggaagaaaatagagggactctgatatacggggaagaaaactcttctttattagaaataaacatgccaccccaggcagtggtggcaaagaagcagacgaatctctgtgagtcggagggcagcctgatctacagagtgagtttgagtccagccaggactacacagagaaactgtcttaaaaaccgtagacaggcagacagatgaatgagcatgcctttcctcctacctagccgactcagggggatgtaaaatgattgtaataaaaatgtttaaagacttttaggaacatgtctttcaggcctagataagtaactgtgccacacagctgacttgtacctttgtgtacttctctagtgccgggtcagctctgtgatgttacagagactgtcctcttgctgacatttgtcccctgctcctgtgtagccccactttcctcactcatctttccgtacatccataggtattaattgagaccctttgtgccctgggagctcagtgccgctggtctgcctgcaacatctattcaactcagaatgaagtagctgcagcactggctgaggctggtgagttccattttttttttctaccaactttgcctcaagtaattaaatacaacacagcagaattgtcattataccttgtatatctggggtctataaatttgggcagtcttaaaacaacagagcctggatataaagcttaatggtggagtagttccctggcatgtagtgcaacagtgaattcgatccccattatcatgacaggggtgaagagaacaggaagaaatgtatactaaaaccgaggtatactttaaaacaaaaaccacaaacacctaaggaaatactcaattctaatgttaaatgaatgaaggcagaatagtaaatgtgagctcagctggtctcttctccccaaaagtcttaatcaattttgagagaacactgtaggtagtgtcttttatattgatagttaatggattatttgcaatttggagtatactaaaatatatgtgctataagttttaattaaaaataaaataatttttattgtcaaaatgttcaaaagcaccgggcggtggtggcgcacgcctttaatcccagcacttgggaggcagaggcaggtggatttctgagttcaaggccagcctggtctacaaagtgagttccaggacagccagggctatacagagaaaccctgtctccaaaaaaaaaaaaagttcaaaagcacatccccttacaccttctaagatcttgagaaagttacagaatagactttgtttaattcatttctaatagctgtttattgtagaagatttaagaaatttaaaaaaaaaacaaagatttgacaagcttctctttatatattggtaaaccaaattttagtatacacacatttttttataaccgaaccattccattttgagaagtaatttatcgtgtcactgaaaagtgaccacctcatctcattcatatggccacatgattttacttcttcattttcctaaaatcgcatgtttaaattattctgagacatctgttggtatctagagtgtctagtattggtgttcctaagagtgctctaggaactttgtttttacatacttcattttgcctccttaagatgcagtataaagtataaaattgacctaaaagacagcaatattttatgactccaagatattttttaatatagaaatctcttaatttcttaactgaagtaatttctgaactaaaaagtactattttgacttttattctttaagttttccctcttcggaattgtttcttttccctttcattcttcatatggatctcagaagttggtacatcagaagcaacattaattgatgtatttctgtctccgtaggagtcgcggtgtttgcttggaagggcgagtcagaagatgatttctggtggtgcattgaccgctgtgtcaacatggatgggtggcaggctaacatggtaatcattcatgtctactccatagttttactatggatttatttccttttctcccttaaatcatggttccctaaatatatttctttgtgtttggtcttattactggctatattgctaaagtgttcataacacaatacctactcggtatggatctaagatgcacaaggccctggattcaatatccagcaccaataagaaagaaaggaaggatggaaagaaaagaaaagaaatgaaagcaaggtctgagaagtgaataggaaggtgggggaagggtaagctgagatggacacatgactcaaacacagcagaataaacagaaggaaatgatgtggtccctctttggagtaggagtcttatttcttttcttcccactccacttctccttttgcattcaaaggagccatatatttcaactagactgaaaatgagcccacgacttgtaatacttatttactgtagctgggcatgaccaaatgcctataagcccagcatttgagaggctgaaacaggaggagtccttgagctcaggaattttagactagcctgagcaacatagtaagttccagtctcaaaaaagagctttccactgtagcctagaactcatttaggatttcttttaaccattttacaccaacagatttaactatatttttactgtctaagcctattctgcagagcgttaagaagtgctctttcaaaatatccagggctctctcactagtgtagaaaatgggctcaaagttccccctagctctagcagcctgaatgcccatagaagtcttctggctgttctgtagatcctggatgatgggggagacttaacccactgggtttataagaagtatccaaacgtgtttaagaagatccgaggcattgtggaagagagcgtgactggtgttcacaggtaacagattctggagtagctgccccttgtgtccttgcctcatcaaattcttctggtctctcctagactttagggctgtgtttcagacttaaaataaccaagtgggagtgtcttcattagataaaaccgagtttttacttttgcaaaatcaaagtgaccataaaaggaattttgaaggaatttttttaaaattctaattgacatgtttttaatctctcccatttgctccactgagtaactaattaacagtatttgggggtaagggtgtagagaggaatgtgtcttggaaaacttgtgctcaatgttgtattataatttttatattttaaatatggagggaaaagtctattgaaaaactacaggtgatgcaacagaaagtaaaactggagatagactcctggcttggaggaagtaactaaatggtatctaggactattgctctgcgggcacagtgtgcgtggttgctctcacagacacagtaaaccacatcagaaccggtgctgtctttgtttacctgaagtaaacatgagtatttaaagtcagaacagtgctgccctaaggttacaatgctccctttgcagaagcagcagtaaattgcttcaactgaaaagttttttatttttcgtttaaaattaaaggataaaatagaaggaaatgagttgaggacaagagactagtcacgtttccaaagaacttctgggcataaataagactaaggagggcagcatggagcttgaagagacagaagcagacttgctatttattgtttataacctaactagttgtgtgtttgttttgtggcttcttctcccttttaggctgtatcagctctccaaagctgggaagctctgtgttccagccatgaatgtcaatgattctgttaccaaacagaagtttgataacctgtactgctgccgagaatccattttggatgggtatgttgaaatgtataccttcagatttgcagggactagtagtgggatttgtttgacgtatcagtttggtacctatagcttgcccttgtagtctccctcctttacatgttattaaaggagcccacttcacagtgatctcagccagctttccattgaatttctcctgggcactgccctacacttgaatttggtacaaagggactgagttgtaaatgatgtttgagatgagcccttgtctgtttccacagcctgaagaggaccacggatgtgatgtttggtgggaaacaggtggtggtgtgtggctatggtgaggtaaatagttgggcccatatctctgtcttattagaaggctttagagaaacatgggatggagacagtctaggaacactgatgaatgtttttccatgctgtgcttgaaaacagtgggaaagtccttttcatatttgtgagttaaaggcagagtgtactgaattttcatattttagcagtttatcttattgattgatgtcagcattactgcctcccacttaaagccttttatgaaataaaagttaagtgaataagtaacattttatccccatcttatggcttgcatcgtagaaactgaagtgacattatagtcgagtatctttaaactggactaggtcctagggagtgtcattgtcttcagcagtggtccactgaagtccttgggctgctaggtattcagtagacacattggaagtgagttttgccaggtttttatggaataaatcatttgttcttgtttgaacttgttttcataatggtaacaattacgcagagcctccctctccccacctcttgttgtgacttgtctgctaaattatctctaattgaagtttacagcctagtttgggtttctttctgctccagctgggactggagtgttcactggatgatgctgatttaaagaggctaattcctatctcctcaaaagcttgtctcccccaatgatcgtatcttcttagatctggcaacttgggctttcctatattctgcaggtaggaaagggctgctgtgctgctctcaaggcccttggagcaattgtctacataacagaaattgaccccatctgtgctctgcaggcctggtaaggacagagtgctggtaccactcttaggtttgaggtccacgccttgtatttctgagagccggtggctgatgactcactttactgtttgtggagaagtctcccggggaagagtaaggtgctttgttcccttcactcgaagccatttatggggcctcttggcattttctcttgctgtgtagactcgtggggcccggcagaagccagtctgtagttcactatagctcatcatttactatttctgggatggagcattagcttttacttcagaaggggattttgttgtctagctttgtcaacgtgagccacacagcacactttgctttgcttatagtgttgctttcttttcttccagcatggatgggttcagggtggtgaagctgaatgaagtcatccggcaggtggacgttgtaattacttgcacaggtgagtatcagtgctttgctgcatggagaacagtgctaactctcaggttgaatctcagaaggtaagacctgtgctcagtgttagccttagagtgcgttctcagaacttttagcattttccaaaagagctttttaccatggtgatttttcatattagtcaatcttaaggccaaataattgagctcaagcatttctgtacacatctttctacagtgtgtgcgagctctttaatggttagcaaacacctctatgttcccgctggaaacagtggctgttcttgttgcccagtgtctctttctccaaccactctcctttacttttaggaaataagaatgtagtgactcgggagcacttggaccgaatgaaaaatagttgtattgtgtgcaatatgggccattccaacacggagatcgacgtggtaaggctgctctccactacagccaggccgtcctcagcatttatttcactagagccacaaccagctgtctgttcttccttccagaccagcctccgcactccagaactaacatgggagcgtgtacgttctcaggtggaccatgtcatctggcctgatggcaaacgggtcgtccttctagcagaggtaacactcgccgccaccgactgcaggcgtgtgctagatgcacagagtaaggaaggggaagcagtgcagatactagttctgtgttctggtaaattctgtctttggataaagatgtgttttcccaattgtctatcccctaaaagagcaggacaccaagaaaacctccgttttgctaagtgcatgcactgtttccagtccttagccagtttcagccttccactgatttgtaaagaggatgaagcctacagtcctgatctgagtggagtgcagagagctataaagtctttcctgtctgcttgacgttggtgccattagaaccggggaaaggcagtggccaacctgtcttttctcttcccagggccgtttacttaatctgagctgctccacagtccctacctttgttctttccatcacggctacaacacaggtatgtggcaaggtgcctggttttcctccactgagaaggaaatgtggcacacacagatacgggctctgatgagtgtactgcctgttcttactgacctattaccttttctacggcctaagttactgaagttcacattgttcctttgaagtggtatttgtgattgtgtctctgtgcagcaatctgcacagctaaaattatagtgttccagcttagttatatttagtttgttcttttgttagaaactaaacaaaaatatcactttctcttatatcatctagcctttatctcagggcagagccctgggtgcactcagatgaccgatcattgtcatgcttatcctaaagtacttttaaatctagttttattttatgtccttgactaagaaatgcttggttctcctgctctgtgtcccaccttaggctttggcactgatagagctttacaacgccccggagggacgctacaaacaggatgtgtacttgcttcctaagaagatgggtgagtgaaaactgaagcctgcactgaccctagttggctgcatgaccctagttggctgcatgcttttataaaatgattttcctaaagcactagctgtagagtcaggcttctgattattcttctagtgccgggcctcagagacattctattaacaccatgagccttcattcttgtgagaagtaatttaagaatagtactatgtcgtttccagaattgtagtaagaatccaagctaagtaaggaagatgttcttattctcaagtcccttccagagctcaccagacttcccagtgcagctaagtacagcttagaccaaagtgagagaaacagaaacaagttgtattatccatctaatgtcttgattggcaaatcacctccaacccaacctctgtgcctcatgggaccacttacactttgctcatgtggcataaaccctgtcttgtcctttgcctttctacatactggcactcaccccatacttaagccagtccacatgcctggctttacactaaaaaaacagcttgcatattggagatgttggtagttaagagcacttgttacttttgcagaggacccagcccaggctcaggtcccagcagccatttagtgtctcacaaccattttgtactgggtaagggactcagtgcctttttctaggctcaatgaccacgaggcacagatgtggtccaaatatatacatacaggaaaacatgcatacacatgaatttttcttaacttattttatattgttttgttgttgttgtgtattttttatgtgtatgggtgttttgcctacatgtgtatgtgcccacaggcatgcctgttgcctgtcaaagtcagaagagggtatcaaaatgcctggaactgcatttatagatgggtgctgccacatgggtgctggaaatcaaacctgagtcttctaaaagaacagacagtgctttaactgctgagtcatgtcaatactgtgtagtgttttgttgttgtaagaaattatattcataaaagttagggaacaatattaaatatgtgccttagaaatccatcagtcaaatcatgaaccataaaaatgaatccaataggcttaaaagagcctcctgtcattgacatgtatttaaggtagggttcctaaagccacatatcttatatgtgatttgacgacagtttaatatagctaaagaggttgccacaggaatctgggtgtagtatatgacatgattacccatgtaactactcaagcgaagccaaaatctgaggggaaagtacaagacacccattcgccaacctggtacatggccctctgtttgcctagtatatatgcttaaaagaaataatttgccagaaatacagctttgaaagctttctcaattaagcagatatttcaagttgtgacttctttcaggattgttccattcctcttctgttcctgagtagttttaaggggtgggcggctgtaccctaaaggcatgtctgacaagcctactgcccttaccactttcctcttcccagatgaatatgttgccagcttgcacttaccatcatttgatgcccacctgacagaactgacagatgaccaagcaaagtatctgggactcaacaaaaatgggccattcaaacctaattattacaggtaccttgaaagaaacgcaatgaaaagttcttctctcaatgttagaccaaccagtttagccaggtctcttcagtaaagataagtgtcatgtctgatatttatgagtataaggtcaccacagcaggttagcagggcaatgactaggctgctcaatattgaatgcttgcctagtatagtgaggttcagtccccagctctacagaaaaccaaattaacaaggtagtcagagaaaatgtgtatctaccaataaatgtaaagaactaagtatcttaaggttttagagaaggtgagcctcacctcaggagaaacccatgaacgaatccccagtctgggctgggggaagcttgggaagatgctgttggtgatctcagaaggcatgtttacatgtctgtctttctctttcagatactaatggacatagtacagtgaccagtccacatgaaccacgcaactctaatagagtattttttaagataacttttattttcttcttattactttccttttgattttttttttctatcatttcattgttgttttctcatctcatcatttgagttttgcagaccacacaggaacttgctccatagctctttaggtgaaactgaggtcaaaggtttctcacccaagtcactaaaaggggttaactctgctgcccagaaagttgattctttaaccatttctgggaactttgatcgtatttagttaccttattaacagaaaatgctaaggcatcttctatgtggaacaatctacagtgtctaaattgccttaaaagagcctgttcctagctgctggaactagtgctctttcacttcttcagaggagccaggatggtacttcccagccaggtaggttagatgtaggtggtgcatgtcagcttcccatagacactctaagccctgtttcctgtgtaaggtgggcatgtctggcagagatgcgttgcttgttcaactcagtaggttcacttgggtttgtagtccagccttccaccagtctctctcattgttctagttttaactaaactgcattctagcaagttgaattttgtcccttagcatttatttctgttttatttcttttaattaagaaaaaaaattattgttaaccctgaggaacagctcaggtggttagttaccaacatctcgtttgaacctttgaagcaaagagtgaagagttgaggctagaaaaatactagtctcagactctgatgtttcc

SEQ ID NO: 6 - protein, 530 aa, Mouse; identical to SEQ ID NO: 2

SEQ ID NO: 7 - DNA, 2431 bp, Mouse
  5′UTR (1)..(239)
  CDS (238)..(1827)
  3′UTR (1828)..(2431)
ctgcgtgctgttgcttctggttctgtggccgccgtcgctgtccggcaggctccggtctcggagccgacgaggcgcgcgagcgcgggtcccagcccttcggaagcccaagcagctcggcgcggggcctggcgggaaggcgggcgagcgcgtggccgagggcgccgcgcggacgggcgggcgcccgtgagggaaagaggcgggggcggcgggttagccgcgggccgggccggccgggggatgtcgatgcctgacgcgatgccgctgcccggtgtcggggaggagctgaaacaggccaaggagatcgaggacgccgagaagtactccttcatggccacggtcaccaaggctcccaagaagcaaatccagtttgctgatgacatgcaagagttcaccaaattccctactaagactggccggagatctttgtctcgttccatctcacaatcctccacagacagctacagttcagctgcatcctatacagatagctctgatgatgaggtttcccctcgagagaagcagcaaaccaactcgaagggcagcagcaatttctgtgtgaagaacatcaagcaggcagagtttggacgccgggagattgagattgcagagcaagacatgtctgctctgatttcactcaggaaacgtgctcagggagagaagcctttggctggtgctaaaatagtgggctgtacgcacatcacggcccagacagcggtattaattgagaccctttgtgccctgggagctcagtgccgctggtctgcctgcaacatctattcaactcagaatgaagtagctgcagcactggctgaggctggagtcgcggtgtttgcttggaagggcgagtcagaagatgatttctggtggtgcattgaccgctgtgtcaacatggatgggtggcaggctaacatgatcctggatgatgggggagacttaacccactgggtttataagaagtatccaaacgtgtttaagaagatccgaggcattgtggaagagagcgtgactggtgttcacaggctgtatcagctctccaaagctgggaagctctgtgttccagccatgaatgtcaatgattctgttaccaaacagaagtttgataacctgtactgctgccgagaatccattttggatggcctgaagaggaccacggatgtgatgtttggtgggaaacaggtggtggtgtgtggctatggtgaggtaggaaagggctgctgtgctgctctcaaggcccttggagcaattgtctacataacagaaattgaccccatctgtgctctgcaggcctgcatggatgggttcagggtggtgaagctgaatgaagtcatccggcaggtggacgttgtaattacttgcacaggaaataagaatgtagtgactcgggagcacttggaccgaatgaaaaatagttgtattgtgtgcaatatgggccattccaacacggagatcgacgtgaccagcctccgcactccagaactaacatgggagcgtgtacgttctcaggtggaccatgtcatctggcctgatggcaaacgggtcgtccttctagcagagggccgtttacttaatctgagctgctccacagtccctacctttgttctttccatcacggctacaacacaggctttggcactgatagagctttacaacgccccggagggacgctacaaacaggatgtgtacttgcttcctaagaagatggatgaatatgttgccagcttgcacttacccacatttgatgcccacctgacagaactgacagatgaccaagcaaagtatctgggactcaacaaaaatgggccattcaaacctaattattacagatactaatggacatagtacagtgaccagtccacatgaaccacgcaactctaatagagtattttttaagataacttttattttcttcttattactttccttttgattttttttttctatcatttcattgttgttttctcatctcatcatttgagttttgcagaccacacaggaacttgctccatagctctttaggtgaaactgaggtcaaaggtttctcacccaagtcactaaaaggggttaactctgctgcccagaaagttgattctttaaccatttctgggaactttgatcgtatttagttaccttattaacagaaaatgctaaggcatcttctatgtggaacaatctacagtgtctaaattgccttaaaagagcctgttcctagctgctggaactagtgctctttcacttcttcagaggagccaggatggtacttcccagccaggtaggttagatgtaggtggtgcatgtcagcttcccatagacactctaagccctgtttcctgtgtaaggtgggcatgtctggcagagatgcgttgcttgttcaactcagtaggttcacttgggtttgtagtccagccttccaccagtctctctcattgttcta

SEQ ID NO: 8 - protein, 530 aa, Mouse
MSMPDAMPLPGVGEELKQAKEIEDAEKYSFMATVTKAPKKQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPTFDAHLTELTDDQAKYLGLNKNGPFKPNYYRY

SEQ ID NO: 9 - DNA, 13462 bp, Artificial Sequence
  DCAL targeting construct
gatatcaagcttggcgcgccgacgtcgcatgcgtatacccatgggtatacgcggccgcccgcgggaattcgatacagaaggcaatgggacagaatatgtatatttttaaatgcctcagaaaaccaaaagaggggtactggggccctcagttaatctcagaggttagaaagacgggtcttcccatgcacacctaaggtttgctttcttaatgatttgttttcctagtgcttgctgtttacctttgtgtctagttcttcacccttaggagaaaaccttcctgaggaaattaaaagctgttagataggtatgtgaaataacaaagttaataagatgcctggcctaacctctaatttttggaggacccacattaaacgatatggcatagacgcaagacatcgtgtctactaaactatttgaaaagctgctttaaaatagcatcttaaactggcctgaaatttaattgggttttaaagatacaacaaatagacttcagtgtttgtgtgtgttatggtagccagcgtgttcctagaggaatggtgaaagttcacagcaaagccatctgttactattacatggagagttgaagtacatacacagattttgagagagctttagttcttgcatcgatctttcataggatgaagtacttagtgctaagtgaatatgttgactaaaagaatataatgcacgttgtatattgtcctgatcattgtcacttatccataggatcatagtagtgttctagtaggttttcattttaatttcctctaattggatagagagaaggtaaatttgaattaggtatgggaagaggggagggcagctgagctatagtgtgtgtagatcatatattttcagaagtaaactcaactttgggtagtaaaaagtattaagatcaattaaacaaataattacagttatttttcttttgaagtttataacgtttttgggttgcctgtgttccctttgtcttggtaagtactgtaccatcttgtagatcactctgtcactataccaaccataatgaacccaaattattaggtcaagcttttcctaatgggcctcagtgttacagagaactcagtccaaccatcataagttgtgctttaaaatagcacactatactgatatgtactatactggccttcaaatgcagcaagtagacattgtttggatgtgctcgatggaaaatggtcgatgtaccctatccttcctcagatatgctgttaatctgagagtggagccgagttgtacttggggtactttgtctatgtttccttctttcttccttgtacttactaccttatgctaaagcaccccagtgaaaacctaacagttggaggaaatcataacaataagaagtaatacaaaccatgcttttgcaattgtctttgggttttcttttatccaaatgaccatatttttccacttaactactgagtttaacccaaacagaaagctgctattagatctggtttcactagaaaccagaagttggttttggcagcagtatccttatttacagcataaaggtgaacagcatgcgaagattctttccaccaagggaggaacagctgctagctggaaagaacctgtagagttagacccctgggggaaacgggcagagccatggcattcagaagcagctgctgtgagacgagactctgcagcttgtagttcagggtagcagagaagacaaggatatcatgcatctgtgtgctgctggaccaccttacagacagctatccactatgccagtacttgagttaacactgcctacttcaaatgtggacaagatctattatttgagagatggggtgttaggagcactgcacatagtagtaagctacttgccatagtctactatttttagatgggaaagattttgctaagctttagaccgctgataagctttattaatacaaggagagattatgtctattaatctccggttgctgaagaaatatttctaacttgctacaactgcttctctatgttttttatctaaaaatctgcaagtgttactgaagtgccttcccccacctcactgcaacgtaggtggtatctggaatcatgcccagaaccttttgaaaaaaaaagtagaagaagaaggggaggaggaggagagggaatctaacagccaggagaagggagcacttccgaacaggctgcccatgccagcagcagggaaccctgtggaggaagtgctgcctctggagctgttacagccactaaggaactcttgactatatttttttcactgccaaagtaactgcatattaggttggaattcgttgctttggtcatctctccttgatttacatcttgcctattgaagaagtgaaagtgtaattggaaatcctatttagaaggatttagtttttgtttgtttttctttttgaatgcagctatgcatttgaagtacttaaaattaaatttttaacatttaaagtaaaatatcttgggctttatgatgatatttgttaatcatattctgaaatgatatatgtggctcagcttgggttgtaattgtctttgaagcttgacttaatattgtacgagaaaagagttttaaaggtttacttattctgcagatgtttattaaataccttcctgcatgtgtgaggccctgacctgctctgggggtatgtagttcttctttatgtcttcagtgtctgttttcagacttaataaaacaggctctccctttctgtgtcagagttgcctgaagaagaacttccacagttagatggtgaaccgagatgagttgtaaacacgcaggtttctgtctctgatctatttgattctgtcctctgcgtttgtgcttagtgggaaccatgctgaataggaagttcaggacaagccagtgtccccttgtttgctactttatgactgtagagtcctaccagttctgtttttcatttgttcattctaactgccgattgattccagagctacaaaccaaagcttggttcttaggctgcagtgtcaccccagcagagactgtatgtgttgggagaggaggattgtacagctgtagctaatgtagagtctactttttaaaatgtgtcattttgtgtatatggatgatttgcctgtctgcatgtttgtgcacagcatgagttcaataccttaagagaagggagtcgggtcatcttaggctcaagttatagacagttgtgagctgccatattggggctggggatagaactcaggtcctctgggaaagcatccagtgcttttaaccactgaactattaccccagtttctggtttcctcttttctagcttgaaagtgttgatgactgggtcatttgtattatgcagaaaaacttttcacctatgatagtatacagtttcattgatactatttgctaacaacataatttaatagtttctccataggaaccttttcagagctctgagtctgaatccaggttctcaatttatactctttttcaggaatgtgggttttatttcaaattttgtagaaagaggtcatcagaattcatgtgagagttcagtgattctcaacctgtgcattgagacccatttgccaaacttctgtctccaaaattacttacataacggtataattttataattttatgcttgggggtcaccacaaatgaggaactatattatagggtctaaacagtaggaaggctcagaaccactggtttaagccatataattccatatttttacatattactttgtttataaataatactgattacaaactattttcaatttattaaagccagtggtccaaatattctaccatgtagatatggtgacacatacatatagccctaacactcaagttctaatgggctgatacaggagaattgtgagttctgagctagcctaggtcttttttttttttttttaagatttatttatttattatatgtaagtacactgtagctgtcctcagatactccagaagagggcatcagatttcgttacggatggttgtgagccaccatgtggttgctgggatttgaactccggactttcagaagagcagtcagtgctcttaaccactgagccatctcaccagcccctagcctaagtcttttaaaagatcttgtcattaaaaaaatatatattctgcaaatataaaactgcaaataactagttctacctaaaaaataatttattctacctacaaagacctttaaaacttgtcaaattttcttcttttagattcatcctgattagttaggatgtaattaatggagaatttggtgttcgttttaaaacttctctttcatccgtacactctgatttttaaattttttactggtgctacagaactctagggatactacattggttttatgaattagagacgacatagctggctgtaatgtgtttagcgattactagacataccattcaaaagcaaagccatggccagattcagaggaagaatatgctacaggagaaccagatgtgagattatatctgctttctgatagaattgggtgaaaggataaagagtttagtgttagtgtgtaaaagttagataaaagggaagggagaaacttaatgagatgaagaaaatttagtaaaattggatagttactaaaataaatggcttttcagaattgtgttctacctccttctctcccccccacccccagcctagatcttcctaaaataactggaaagagacagtagggttggttttttttttttttaaagatttattttatttatatgagtacactgtctctgtcttcagacataccagaaggacatatcagattccattatagatggttgtgagctaccatgtggttactgggaattgaactcaggacctctgggagatcagtgagtgctcttaactgctgagctagcccaagacaatagttgtaacagagttcagaaagcagcctcttccccgcccttcagatgagagttactgccacagtaagaaccacatggtgacgccgaattttaggaaagttagtgtatggatttggaaaaggaaaagttttcgtacagcaagatggttccaagggtgaagatgcctgcctccaaggctgacaactaagttctatctccaggactcacgtggtgaagagagagcagactcccacagtttgccctctgacttcttgtgtgccaattccgatcatattcaataacccttaatataacttcgtataatgtatgctatacgaagttattaggtctgaagaggagtttacgtccagccaagctagcttggctgcaggtcgtcgacggtatcgataagcttgatatcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctcgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagagcttgcggaacccttaatataacttcgtataatgtatgctatacgaagttattaggtccctcgagagaagcagcaaaccaactcgaagggcagcagcaatttctgtgtgaagaacatcaagcaggcagagtttggacgccgggagattgagattgcagagcaaggtaaaggaggaaatgagcagagggccagaagcacctttgcactgcttatcaggacctggacaatttaagtgtcctggaaaagagtttctcacactttggataaacacttctctctatatggaaactgagccttaggattcttgggtgtgttttgttttgttttcttttttttctgttcccagtgtatggtaattacagagtggtttctgttctcctcatcatgacaccatactcacaggtctacagtgcatagtcagcaagtgttggatcataacatagtggttatttgtgtgggatatttttctagctgccaatacagagacccggatttcccagaagcaggtgatatgataggtttctcacctaaggttgtggtagcctggggtctcattaagtcctagactccctcagctggaacggaatacaggtgactgacagtcaactgcatcttaatttctctcttcctctacacttctccaagggtctctgctcttctgtgttttggtttttttcatgttgaagttgtgtttcttactccgatcttgacaagctcaggtaagataatagagaaaaatagaaatgtcttccactgagttcacagtcatccctcctggactgatgaagacatatggaagtctgtttattttggccagtttggaaagtttaattctttctctgctcaggcctttgtatgtataaagcagaagcaagacccgccaattgtacttacatcataacctccagttactccattggtgaaacatctagtcatgagggaaatatgtttgatattttctaccccatttcgccatgggtcagcagacctaacactgctcgtggctggacaggagcgaatggtgttggtagtgtggacctggctcagatcctctgttcttccacagacatgtctgctctgatttcactcaggaaacgtgctcagggagagaagcctttggctggtgctaaaatagtgggctgtacgcacatcacggcccagacagcggtgagtttgttggaagaaaatagagggactctgatatacggggaagaaaactcttctttattagaaataaacatgccaccccaggcagtggtggcaaagaagcagacgaatctctgtgagtcggagggcagcctgatctacagagtgagtttgagtccagccaggactacacagagaaactgtcttaaaaaccgtagacaggcagacagatgaatgagcatgcctttcctcctacctagccgactcagggggatgtaaaatgattgtaataaaaatgtttaaagacttttaggaacatgtctttcaggcctagataagtaactgtgccacacagctgacttgtacctttgtgtacttctctagtgccgggtcagctctgtgatgttacagagactgtcctcttgctgacatttgtcccctgctcctgtgtagccccactttcctcactcatctttccgtacatccataggtattaattgagaccctttgtgccctgatcactagtgaattcgcggccgcctgcaggtcgactctagaggatccccgggtaccgagctcgaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacctgcagcgacccgcttaacagcgtcaacagcgtgccgcagatcttggtggcgtgaaactcccgcacctcttcggccagcgccttgtagaagcgcgtatggcttcgtacccctgccatcaacacgcgtctgcgttcgaccaggctgcgcgttctcgcggccataacaaccgacgtacggcgttgcgccctcgccggcaacaaaaagccacggaagtccgcctggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgttgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttatgggctgcttgccaatacggtgcggtatctgcagggcggcgggtcgtggcgggaggattggggacagctttcgggggcggccgtgccgccccagggtgccgagccccagagcaacgcgggcccacgaccccatatcggggacacgttatttaccctgtttcgggcccccgagttgctggcccccaacggcgacctgtataacgtgtttgcctgggctttggacgtcttggccaaacgcctccgtcccatgcatgtctttatcctggattacgaccaatcgcccgccggctgccgggacgccctgctgcaacttacctccgggatggtccagacccacgtcaccaccccaggctccataccgacgatctgcgacctggcgcgcacgtttgcccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccacgggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgccgccggacgaactaaacctgactacggcatctctgccccttcttcgctggtacgaggagcgcttttgttttgtattggtcaccacggccgagtttccgcgggaccccggccaggacctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgggggtaccgagctcgacgaattctctagatatcgctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagtttacgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatctcacggatggcatgacagtaagagaattatgcagtgctgccataaccatgagtgataacactgcggccaacttacttctggcaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggatcacttctgcgctcggccctcccggctggctggtttattgctgataaatctggagccggtgagcgtgggtctcgcggtatcattgcagcactggggccagatggtaagccctcccgcatcgtagttatctacacgacggggagtcaggcaactatggatgaacgaaatagacagatcgctgagataggtgcctcactgattaagcattggtaatgagggcccaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaccaccgctggtagcggtggtttttttgtttgcaagcagcagattacgcgcagaaaaaaaggatctcaagaagatcctttgattttctaccgaagaaaggcccacccgtgaaggtgagccagtgagttgattgcagtccagttacgctggagtctgaggctcgtcctgaat

(19) United States
(12) Patent Application Publication
(10) Pub. No.: US 2005/0044582 A1
(43) Pub. Date: Feb. 24, 2005

(54) Novel polynucleotides and uses therefor

(75) Inventors: Masato Kato, Queensland (AU); Nicola Zoe Angel, Queensland (AU); Benjamine Jay Cooper, Queensland (AU); Derek Nigel John Hart, Queensland (AU)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: The Corporation of the Trustees of the Order of the Sisters of Mercy in Queensland

(21) Appl. No.: 10/876,285

(22) Filed: Jun. 24, 2004

Related U.S. Application Data
(63) Continuation-in-part of application No. PCT/AU02/01761, filed on Dec. 24, 2002.

(30) Foreign Application Priority Data
Dec. 24, 2001 (AU) PR9741/01

(51) Int. Cl. A01K 67/027; C07H 21/04; C07K 14/705

(52) U.S. Cl. 800/14; 536/23.5; 530/350; 435/69.1; 435/320.1; 435/325

(57) ABSTRACT

The present invention discloses the genomic structure and sequence of DCAL. It also provides methods for producing a genetically modified non-human animal model having altered DCAL gene function as well as methods for using this model in the study of the immune system, and particularly DC function, and in screening for biologically active agents that modulate DCAL function. The present invention also discloses the use of such modulatory agents in methods for modulating immune responses, and in compositions for treating and/or preventing DCAL-related conditions. Also disclosed is the use of the genetically modified non-human animal model of the invention in the study of brain physiology and more particularly neuronal cell function.

### RELATED APPLICATIONS

This application is a continuation-in-part of International Application No PCT/AU02/01761, filed Dec. 24, 2002, designating the United States and published in English, and which claims priority to Australian Provisional Application No. PR 9741, filed Dec. 24, 2001.

### FIELD OF THE INVENTION

This invention relates generally to sequences expressed by dendritic cells. More particularly, the present invention relates to genomic sequences encoding dendritic cell expressed S-adenosyl homocysteine hydrolase-like molecule (DCAL), and to their use in the production of a genetically modified non-human animal model having altered DCAL gene function. The genetically modified animal may be either homozygous or heterozygous for a disruption in the endogenous DCAL gene.

Bibliographic details of various publications referred to in this specification are collected at the end of the description.

### BACKGROUND OF THE INVENTION

Dendritic cells (DC) are highly effective antigen presenting cells that mediate vital interactions between the host environment and the cells of the immune system, thereby initiating and directing the primary immune response and maintaining tolerance (Hart 1997; Steinman 1991). DC develop from one or more bone marrow precursors (Egner et al. 1993; Szabolcs et al. 1996), and functionally immature DC migrate via the bloodstream to tissues where they may reside for periods ranging from days to months depending on the type of DC and tissue. At this tissue surveillance stage, immature DC are proficient at taking up antigen, but are comparatively poor at co-stimulating T lymphocyte responses (Romani et al. 1989). Following exposure to “danger signals”, the presence of foreign antigen, or perhaps through normal differentiation and trafficking processes, DC leave the tissues, cross the lymphatic endothelium and migrate along cytokine and chemokine gradients to the T lymphocyte areas of lymph nodes (Cumberbatch and Kimber 1992; Sozzani et al. 1995). Within the lymph node, DC recruit and stimulate naive T lymphocytes in a highly regulated process, controlled in part by reciprocal signals derived from specifically activated T lymphocytes (McLellan et al. 1999; McLellan et al. 1996). There is also evidence that DC interact directly with B cells and contribute to the humoral response (Fayette et al. 1997). These “mature” DC lack the capacity to take up antigen but have strong costimulatory activity. Subsequent to their interaction with T lymphocytes, DC disappear from the lymph node, possibly through an apoptotic process (Ingulli et al. 1997). It is clear that DC mediate different functions during their differentiation or maturation and that the molecular control of these events must be strictly regulated in order to optimise the immune response to foreign antigens, yet maintain tolerance to self antigens.

The different stages of DC ontogeny are characterized by the expression of particular molecules. DC express cytokines, chemokine receptors and costimulatory molecules in a time- or differentiation state-dependent manner. The appearance of other cell surface molecules, such as CMRF-44 (Hock et al. 1994), CMRF-56 (Hock et al. 1999) and CD83 (Zhou and Tedder 1995), define differentiated/activated DC populations. The expression of the transcription factor relB coincides with human blood or tissue DC activation (Clark et al. 1999) and inactivation of relB in mice preserves LC, but blocks subsequent DC immune function (Burkly et al. 1995). Blood DC acquire effective antigen uptake and processing capabilities only after in vitro culture (Mannering et al. 1998) and is characterized by the induction of several molecular pathways, including the lysosomal processing pathway associated with DC-LAMP expression (de Saint-Vis et al. 1998). Thus, it is clear that DC selectively express different molecules appropriate to their stage of differentiation and function. However, little is known of the complex regulatory pathways required to control the expression of these molecules at an appropriate time.

In work leading up to the present invention and described in International publication No. WO 98/14562, a differential display technique, based on the RNA arbitrarily primed polymerase chain reaction (RAP-PCR) method (Welsh et al. 1992), was used to identify genes with restricted expression patterns in DC populations. Low cell numbers, cell purity and unknown heterogeneity are all factors which can confound differential display techniques. To overcome these problems, the inventors applied the RAP-PCR method to the cell lines L428 (a Hodgkin's disease-derived cell line with many phenotypic similarities to differentiated/activated peripheral blood DC (McKenzie et al. 1992) and U937 (a monocytoid cell line). Using this technique, a cDNA was identified that was predicted to encode a protein containing a domain similar to the methylation pathway enzyme S-adenosyl-L-homocysteine hydrolase (AHCY) (Chiang et al. 1996; Coulter-Karis and Hershfield 1989; Ueland 1982). The mRNA for this molecule is expressed abundantly in differentiated/activated DC, but at very low or undetectable levels in other normal leukocyte populations. The molecule, which was designated DC-expressed AHCY-like molecule (DCAL), appears to have peak mRNA expression during DC antigen uptake and migration from the peripheral tissues. The inventors have also determined from recent experiments that DCAL is expressed by fresh and cultured peripheral blood DC and freshly isolated skin Langerhans cells (LC), but is poorly or otherwise not expressed in other peripheral blood mononuclear cells (PBMC) including monocytes, T- and B-lymphocytes, NK cells and granulocytes. It has also been determined that DCAL mRNA expression is associated with the antigen uptake and lymphatic migration stages of DC function, but not with antigen presentation to T lymphocytes, or post-presentation events (e.g., apoptosis). In view of its restricted expression in normal leukocytes and its expression pattern in DC at different stages of function/differentiation, the inventors consider that DCAL has an important regulatory role in DC function.

Surprisingly, the inventors have also determined that DCAL is expressed at relatively high levels in adult brain tissue but at relatively low levels in fetal brain tissue. Accordingly, it is predicted that DCAL expression is likely to be related to the function of neuronal cells rather than to the differentiation of this cell type and that DCAL is likely to have an important role in neuronal cell function.

### SUMMARY OF THE INVENTION

The present invention is predicated in part on the determination of the genomic structure and sequence of DCAL, which make possible a range of products and methods, including the production of a genetically modified non-human animal model having altered DCAL gene function. Such a model is useful inter alia for studying the immune system, and particularly DC function, and in screening for biologically active agents that modulate DCAL function and that might be used, therefore, in methods for modulating immune responses, and in compositions for treating and/or preventing DCAL-related conditions. The genetically modified non-human animal model of the invention may also be useful in the study of brain physiology and more particularly neuronal cell function.

Accordingly, in one aspect of the present invention, there is provided an isolated polynucleotide comprising a nucleotide sequence which corresponds or is complementary to at least a portion of the sequence set forth in SEQ ID NO: 1 or 5.

Suitably, the portion is at least 18 nucleotides, preferably at least 25 nucleotides, more preferably at least 50 nucleotides, even more preferably at least 100 nucleotides, even more preferably at least 150 nucleotides, even more preferably at least 200 nucleotides, even more preferably at least 300 nucleotides, even more preferably at least 400 nucleotides, and still even more preferably at least 500 nucleotides in length. Typically, the portion includes at least one intron and/or at least one exon, or a segment thereof.

Preferably the nucleotide sequence is a variant having at least 60%, suitably at least 65%, preferably at least 70%, more preferably at least 75%, even more preferably at least 80%, even more preferably at least 85%, even more preferably at least 90% and still even more preferably at least 91%, 92%, 93%, 94%, 95%, 96%, 97%, 98% or 99% sequence identity to at least a portion of the sequence set forth in SEQ ID NO: 1 or 3. In another embodiment, the variant is capable of hybridizing to at least a portion of the sequence set forth in SEQ ID NO: 1 or 5 under at least low stringency conditions, preferably under at least medium stringency conditions, and more preferably under high stringency conditions.

In another aspect, the invention contemplates a vector comprising the polynucleotide as broadly described above.

In a preferred embodiment, the vector is a DNA targeting vector.

In yet another aspect, the invention envisions a host cell containing a vector as broadly described above.

In still yet another aspect, the invention encompasses a non-human genetically modified animal having altered DCAL function. In one embodiment, the genetically modified animal has a partial or complete loss of function in one or both alleles of the endogenous DCAL gene. In this embodiment, the genetically modified animal preferably comprises a disruption in at least one allele of the endogenous DCAL gene. Suitably, the disruption has been introduced into its genome by homologous recombination with a DNA targeting construct in an embryonic stem cell such that the targeting construct is stably integrated in the genome of the animal, wherein the disruption of the DCAL gene results in an inability of the animal to produce a functional DCAL or detectable levels of DCAL.

The present invention further provides a method for disrupting a DCAL gene in a cell, comprising:
providing a first polynucleotide having a sequence comprising: i) at least a portion of a non-human DCAL gene; and ii) a second polynucleotide capable of disrupting the non-human DCAL gene; and
introducing the first polynucleotide into a non-human cell under conditions such that the first polynucleotide is homologously recombined into at least one of the naturally occurring alleles of the DCAL gene in the genome of the cell to produce a cell containing at least one disrupted DCAL allele.

In a preferred embodiment, the method for disrupting a DCAL gene in a cell is a method for producing a non-human genetically modified animal containing at least one disrupted DCAL allele.

In another preferred embodiment, the non-human genetically modified animal containing the homologously recombined polynucleotide is further characterized by expressing reduced or undetectable levels of DCAL.

In an alternate preferred embodiment, the non-human genetically modified animal lacks the ability to produce functional DCAL.

The genetically modified animal and cells derived therefrom are useful for screening biologically active agents that modulate DCAL function. The screening methods are of particular use for determining the specificity and action of drugs that may interact with DCAL. The genetically modified animal is useful as a model to investigate the role of DCAL in dendritic cell function and modulation of immune responses. It may also be useful as a model to investigate the role of DCAL in brain physiology and more particularly in neuronal cell function.

Thus, in another aspect, the invention features a process for screening a candidate agent for the ability to specifically modulate DCAL function, comprising:
a. administering a candidate agent to a genetically modified non-human animal as broadly described above and to a corresponding wild-type non-human animal; and
b. comparing the immune response of the genetically modified animal to the immune response of the wild-type animal,

wherein a substantial absence in modulation of immune response in the genetically modified animal and a substantial modulation of immune response in the wild-type animal indicates that the candidate agent specifically modulates DCAL function.

In another aspect, the invention extends to a process for screening a candidate agent for the ability to specifically modulate DCAL function, comprising:
a. exposing first DC from a genetically modified non-human animal as broadly described above and second DC from a corresponding wild-type non-human animal to a candidate agent; and
b. comparing the individual immune responses of the first DC and of the second DC,

wherein a substantial absence in modulation in the immune response of the first DC and a substantial modulation in the immune response of the second DC indicates that the candidate agent specifically modulates DCAL function.

In a further aspect, the invention provides a modulatory agent which modulates an immune response, wherein the agent is identified by a process as broadly described above.

In another aspect, the invention contemplates a method for modulating an immune response, the method comprising administering to a patient in need of such treatment a modulatory agent as broadly described above for a time and under conditions sufficient to modulate the immune response.

In a preferred embodiment, the agent reduces or suppresses the immune response.

In another aspect, the invention resides in a composition for treatment and/or prophylaxis of a condition associated with expression or activation of DCAL, the composition comprising an agent as broadly described above, which reduces the level and/or functional activity of DCAL, together with a pharmaceutically acceptable carrier.

According to another aspect of the invention, there is provided a method for treatment and/or prophylaxis of a condition associated with expression or activation of DCAL, the method comprising administering to a patient in need of such treatment a therapeutically effective amount of an agent as broadly described above for a time and under conditions sufficient to reduce the level and/or functional activity of DCAL.

Suitably, the condition is selected from an allergy, autoimmune disease, or transplant rejection.

In still yet another aspect, the invention encompasses the use of the genetically modified animals and modulatory agents as broadly described above in the study of immunity and more particularly DC function.

The invention also encompasses the use of the genetically modified animals and modulatory agents as broadly described above in the study of brain physiology and more particularly neuronal cell function.

### BRIEF DESCRIPTION OF THE DRAWINGS

FIG. 1A is a schematic representation of the full length DCAL cDNA clone 211(1)B with the region corresponding to DCAL domains (see keys below). The positions of the RAP-PCR product DD4b5.3 (DD4b5.3 PCR frag) is indicated by bars. Positions of the primers used for analysis are indicated by arrows.

FIG. 1B is a diagrammatic representation showing the DNA sequence of DCAL cDNA. The putative start codon is boxed. The area corresponding to AHCY-like domain is shaded. The in-frame stop codon is indicated by an asterisk.

FIG. 2A is a photographic representation showing agarose gel electrophoresis of the first and second PCR products using RNA ligase mediated (RLM) 5′-RACE of RNA from L428 cells.

FIG. 2B is a photographic representation showing the identification of DCAL 5′-RACE clones. The 5′-RACE product cloned into pGEM-T easy vector was PCR amplified using SP6 and T3 primers, and the PCR products fractionated with agarose gel. The positions for DNA standards are shown on the left.

FIG. 2C is a diagrammatic representation showing the DCAL transcription start sites, which are indicated on the DCAL cDNA (clone 211(1)B, capital letters) and the genomic DNA sequence (small letters) corresponding to 5′-proximal region of the cDNA. The numbers indicate the relative positions of transcription start sites to the 5′-end of the DCAL cDNA.

FIG. 3A is a photographic representation showing the expression of DCAL in a panel of normal leukocyte populations. Total RNA from FACS-purified leukocytes was subjected to RT-PCR for DCAL (upper panel) and beta 2 microglobulin (b2m) (lower panel). DCAL RT-PCR was subsequently probed with a DIG-labeled DCAL-specific internal primer.

FIG. 3B is a photographic representation showing the expression of DCAL in a panel of DC lineage populations. Total RNA from FACS-purified DC was subjected to RT-PCR for DCAL (upper panel) and b2m (lower panel).

FIG. 4 is a photographic representation illustrating the expression of DCAL in monocyte differentiation to Mo-DC. Monocytes were cultured in the presence of GM-CSF and IL-4 in the presence or absence of a CpG oligonucleotide or LPS. Total RNA from various stage of Mo-DC differentiation was subjected to RT-PCR for DCAL (upper panel) and GAPDH (lower panel).

FIG. 5A is a diagrammatic representation showing the amino acid sequence comparison between respective hydrophilic domains of DCAL (524 aa), KIAA0828 (619 aa), Drosophila bithorax-complex derived 58K putative protein (Dros. 58K, 504 aa) and human AHCY (432 aa). Identical amino acids to those of DCAL are indicated by dashes (-). The conservatively replaced amino acids are shaded. The conserved cysteines are indicated by asterisks (*). In the hydrophilic domain, conserved serines and threonines are indicated by closed diamonds (▾).

FIG. 5B is a diagrammatic representation showing the amino acid sequence comparison between respective AHCY-like domains of DCAL (524 aa), KIAA0828 (619 aa), Drosophila bithorax-complex derived 58K putative protein (Dros. 58K, 504 aa) and human AHCY (432 aa). Identical amino acids to those of DCAL are indicated by dashes (-). The conservatively replaced amino acids are shaded. The conserved cysteines are indicated by asterisks (*). The conserved (O) or conservatively replaced (●) amino acids required for the binding of ribose ring of adenosine homocysteine are also indicated. The conserved NAD⁺ binding motif is indicated by a bracket. The conserved amino acid required for binding of NAD⁺ are indicated by open diamonds (⋄). An amino acid deletion found in DCAL, KIAA0828 and Drosophila. 58K, but not in AHCY is indicated by an arrow.

FIG. 6A is a schematic representation depicting the gene structure of DCAL (partial), KIAA0828 (partial) and AHCY gene. The filled boxes indicate exons. Exons encoding the AHCY-like domain of DCAL and KIAA0828 are indicated by brackets.

FIG. 6B is a schematic representation showing exon-intron junctions in DCAL (partial), KIAA0828 (partial) and AHCY on their cDNA coding regions. The AHCY-like (for DCAL and KIAA0828) or ACHY (for AHCY) domains are indicated by shaded boxes. The positions of exon-intron junctions are indicated by arrows. The numbers under the arrows indicate the phases of exon-intron junctions.

FIG. 7A-G is a diagrammatic representation showing the construction of one embodiment of a targeting vector for generating a DCAL knockout mouse.

FIG. 8 is a diagrammatic representation illustrating a Southern blot analysis, which distinguishes between transgene and wild-type genomic DNA.

FIGS. 9A-I depicts an annotated sequence of the DCAL targeting vector whose construction is shown in FIG. 7.

FIG. 10 is a diagrammatic representation showing the construction of one embodiment of a conditional knockout vector for generating a conditional DCAL knockout mouse.

### BRIEF DESCRIPTION OF THE SEQUENCES

TABLE 1

| SEQUENCE ID | | |
|---|---|---|
| NUMBER | SEQUENCE | LENGTH |
| SEQ ID NO: 1 | Human DCAL genomic DNA | 44096 nts |
| SEQ ID NO: 2 | Human DCAL polypeptide encoded by | 530 aa |
| | SEQ ID NO: 1 | |
| SEQ ID NO: 3 | Polynucleotide corresponding to human | 2655 nts |
| | DCAL cDNA | |
| SEQ ID NO: 4 | Human DCAL polypeptide encoded by | 530 aa |
| | SEQ ID NO: 3 | |
| SEQ ID NO: 5 | Mouse DCAL genomic DNA | 33000 nts |
| SEQ ID NO: 6 | Mouse DCAL polypeptide encoded by | 530 aa |
| | SEQ ID NO: 5 | |
| SEQ ID NO: 7 | Polynucleotide corresponding to mouse | 2431 nts |
| | DCAL cDNA | |
| SEQ ID NO: 8 | Mouse DCAL polypeptide encoded by | 530 aa |
| | SEQ ID NO: 7 | |
| SEQ ID NO: 9 | DCAL knockout construct | 13462 nts |

### DETAILED DESCRIPTION OF THE PREFERRED EMBODIMENT

A. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “agent” is meant a naturally occurring or synthetically produced molecule which interacts either directly or indirectly with a target molecule, the level and/or functional activity of which is to be modulated.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

The term “allogeneic” as used herein refers to cells, tissues, organisms etc that are of different genetic constitution.

By “autologous” is meant something (e.g., cells, tissues etc) derived from the same organism.

The term “complementary” refers to the topological capability or matching together of interacting surfaces of a test polynucleotide and its target oligonucleotide, which may be part of a larger polynucleotide. Thus, the test and target polynucleotides can be described as complementary, and furthermore, the contact surface characteristics are complementary to each other. Complementary includes base complementarity such as A is complementary to T or U, and C is complementary to G in the genetic code. However, this invention also encompasses situations in which there is non-traditional base-pairing such as Hoogsteen base pairing which has been identified in certain transfer RNA molecules and postulated to exist in a triple helix. In the context of the definition of the term “complementary”, the terms “match” and “mismatch” as used herein refer to the hybridisation potential of paired nucleotides in complementary nucleic acid strands. Matched nucleotides hybridise efficiently, such as the classical A-T and G-C base pair mentioned above. Mismatches are other combinations of nucleotides that hybridise less efficiently.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules. Accordingly, the term derivative encompasses molecules that will modulate DC function and/or an immune response.

By “effective amount”, in the context of modulating an activity or of treating or preventing a condition associated with the expression of DCAL is meant the administration of that amount of active to an individual in need of such modulation, treatment or prophylaxis, either in a single dose or as part of a series, that is effective for modulating that activity or for treating or preventing that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The term “foreign polynucleotide” or “exogenous polynucleotide” or “heterologous polynucleotide” refers to any nucleic acid (e.g., a gene sequence) which is introduced into the genome of an animal by experimental manipulations and may include gene sequences found in that animal so long as the introduced gene contains some modification (e.g., a point mutation, the presence of a selectable marker gene, the presence of a loxP site, etc.) relative to the naturally-occurring gene.

The terms “fragment,” “portion” and “segment” are use interchangeably herein to refer to a part, or component of a polynucleotide or polypeptide, i.e., less than the whole polynucleotide or whole polypeptide.

The term “gene” as used herein refers to any and all discrete coding regions of the cell's genome, as well as associated non-coding and regulatory regions. Thus, the term “DCAL gene” is used generically herein to designate DCAL genes, e.g. variants from rat, human, mouse, guinea pig, etc., and their alternate forms. The gene is also intended to mean the open reading frame encoding specific polypeptides, introns, and adjacent 5′ and 3′ non-coding nucleotide sequences involved in the regulation of expression, up to about 1 kb beyond the coding region, but possibly further in either direction. In this regard, the gene may further comprise endogenous (i.e., naturally associated with a given gene) or heterologous control signals such as promoters, enhancers, termination and/or polyadenylation signals. The DNA sequences encoding DCAL may be cDNA or genomic DNA or a segment thereof. The gene may be introduced into an appropriate vector for extrachromosomal maintenance or for integration into the host.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state.

The term “knock-in” generally refers to a heterologous or foreign gene or part thereof that has been inserted into a genome through homologous recombination. The knock-in gene or gene part may be a mutant form of a gene or gene part that replaces the endogenous, wild-type gene or gene part. Such mutations include insertions of heterologous sequences, deletions, point mutations, frameshift mutations and any other mutations that may prevent, disrupt or alter normal gene expression. Thus, a “knock-in” animal, as used herein, refers to a genetically modified animal in which a specific gene or part thereof is replaced by a foreign gene or DNA sequence. A “conditional knock-in” refers to a heterologous or foreign gene or part thereof that has been inserted into a genome through homologous recombination and that is expressed at a designated developmental stage or under particular environmental conditions. A “conditional knock-in vector” is a vector including a heterologous or foreign gene or part thereof that can be inserted into a genome through homologous recombination and that can be expressed at a designated developmental stage or under particular environmental conditions.

By “knock-out” is meant the inactivation or loss-of-function of a gene, which decreases, abrogates or otherwise inhibits the level or functional activity of an expression product of that gene. A “knock-out” animal refers to a genetically modified animal in which a gene is inactivated or loses function. A “conditional knock-out” refers to a gene that is inactivated or loses function under specific conditions, such as a gene that is inactivated or loses function in a tissue-specific or a temporal-specific pattern. A “conditional knock-out vector” is a vector including a gene that can be inactivated or whose function can be lost under specific conditions.

The term “loss-of-function,” is art recognized and, with respect to a gene or gene product, refers to mutations in a gene which ultimately decrease or otherwise inhibit the level or functional activity of an expression product of that gene. For example, a loss-of-function mutation to a gene of interest may be a point mutation, deletion or insertion of sequences in the coding sequence, intron sequence or 5′ or 3′ flanking sequences of the gene so as to, for example, (i) alter (e.g., decrease) the level gene expression, (ii) alter exon-splicing patterns, (iii) alter the activity of the encoded protein, or (iv) alter (decrease) the stability of the encoded protein. For example, the term “loss-of-function,” as it relates to an alteration of the DCAL gene, refers to a diminishment or abrogation in the functional activity of the DCAL gene expression product when compared to its functional activity in the absence of the alteration.

By “modulating” is meant increasing or decreasing, either directly or indirectly, the level and/or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

The term “operably linked”, with reference to a polypeptide-encoding polynucleotide, means that transcriptional and translational regulatory elements are positioned relative to the polypeptide-encoding polynucleotide in such a manner that the polynucleotide is transcribed and the encoded polypeptide is translated. Alternatively, with reference to a non-polypeptide encoding polynucleotide (e.g., polynucleotides encoding antisense molecules or ribozymes) the term “operably linked” refers to transcriptional regulatory elements being positioned relative to the non-polypeptide-encoding polynucleotide in such a manner that the polynucleotide is transcribed.

The term “patient” refers to patients of human or other animal and includes any individual it is desired to examine or treat using the methods of the invention. However, it will be understood that “patient” does not imply that symptoms are present. Suitable animals that fall within the scope of the invention include, but are not restricted to, primates, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., kangaroo, koala, foxes, deer, dingoes), aves (e.g., chicken, geese, duck, emu, ostrich), reptile or fish.

By “pharmaceutically-acceptable carrier” is meant a solid or liquid filler, diluent or encapsulating substance that may be safely used in topical or systemic administration.

The term “polynucleotide” or “nucleic acid” as used herein designates mRNA, RNA, cRNA, cDNA or DNA. The term typically refers to oligonucleotides greater than 30 nucleotides in length. Polynucleotide sequences are understood to encompass complementary strands as well as alternative backbones described herein.

The terms “polynucleotide variant” and “variant” refer to polynucleotides displaying substantial sequence identity with a reference polynucleotide sequence or polynucleotides that hybridize with a reference sequence under stringent conditions that are defined hereinafter. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions) as described hereinafter. Accordingly, polypeptide variants as used herein encompass polypeptides that will modulate DC function and/or an immune response.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but may alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotides, although it may contain fewer nucleotides. Primers can be large polynucleotides, such as from about 200 nucleotides to several kilobases or more. Primers may be selected to be “substantially complementary” to the sequence on the template to which it is designed to hybridize and serve as a site for the initiation of synthesis. By “substantially complementary”, it is meant that the primer is sufficiently complementary to hybridize with a target nucleotide sequence. Preferably, the primer contains no mismatches with the template to which it is designed to hybridize but this is not essential. For example, non-complementary nucleotides may be attached to the 5′ end of the primer, with the remainder of the primer sequence being complementary to the template. Alternatively, non-complementary nucleotides or a stretch of non-complementary nucleotides can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another nucleic acid, often called the “target nucleic acid”, through complementary base pairing. Probes may bind target nucleic acids lacking complete sequence complementarity with the probe, depending on the stringency of the hybridization conditions. Probes can be labeled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing.

“Stringent conditions” refers to temperature and ionic conditions under which only nucleotide sequences having a high frequency of complementary bases will hybridize. The stringency required is nucleotide sequence dependent and depends upon the various components present during hybridization and subsequent washes, and the time allowed for these processes. Generally, in order to maximize the hybridization rate, non-stringent hybridization conditions are selected; about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately stringent washing conditions are selected to be about 15 to 30° C. lower than the Tₘ. Highly permissive (low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences. Other examples of stringency conditions are described in section 3.3.

By “substantially complementary” it is meant that a test polynucleotide is sufficiently complementary to hybridize with a target sequence. Accordingly, the nucleotide sequence of the test polynucleotide need not reflect the exact complementary sequence of the target sequence.

By “substantial modulation of the immune response” is meant at least a 5%, more suitably at least a 10%, preferably at least a 20%, more preferably at least a 30%, even more preferably at least a 40%, even more preferably at least a 50%, even more preferably at least a 60%, even more preferably at least a 70%, even more preferably at least a 80%, even more preferably at least a 90%, and still even more preferably at least a 100% change in the strength of a reference immune response.

The term “transgene” is used herein to describe genetic material that has been or is about to be artificially inserted into the genome of a cell, particularly a mammalian cell of a living animal. The transgene is used to transform a cell, meaning that a permanent or transient genetic change, preferably a permanent genetic change, is induced in a cell following incorporation of exogenous DNA. A permanent genetic change is generally achieved by introduction of the DNA into the genome of the cell. Vectors for stable integration include plasmids, retroviruses and other animal viruses, YACs, and the like. Of interest are non-human animals include vertebrates, preferably mammals such as rodents, non-human primates, ovines, bovines, ruminants, lagomorphs, porcines, caprines, equines, canines, felines, aves, etc.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

The term “wild-type” refers to a gene or gene product which has the characteristics of that gene or gene product when isolated from a naturally occurring source. A wild-type gene is that which is most frequently observed in a population and is thus arbitrarily designed the “normal” or “wild-type” form of the gene. In contrast, the term “modified” “variant” or “mutant” refers to a gene or gene product which displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, underscoring or italicizing the name of a gene shall indicate the gene, in contrast to its protein product, which is indicated by the name of the gene in the absence of any underscoring or italicizing. For example, “DCAL” shall mean the DCAL gene, whereas “DCAL” shall indicate the protein product of the “DCAL” gene.

B. Polynucleotides of the Invention

1. DCAL Polynucleotides

The present invention provides an isolated polynucleotide comprising a DCAL gene or portion thereof. In one embodiment, the polynucleotide comprises the entire sequence of nucleotides set forth in SEQ ID NO: 1. SEQ ID NO: 1 corresponds to a 44096-bp human genomic sequence for DCAL. This sequence defines: (1) a first exon comprising a 5′ untranslated region from nucleotide 9 through nucleotide 281 and a portion of the DCAL open reading frame (ORF) from nucleotide 282 through nucleotide 401; (2) a first intron from nucleotide 402 through nucleotide 24262; (3) a second exon, comprising a portion of the DCAL ORF, from nucleotide 24623 through nucleotide 24374; (4) a second intron from nucleotide 24375 through nucleotide 26442; (5) a third exon, comprising a portion of the DCAL ORF, from nucleotide 26443 through nucleotide 26586; (6) a third intron from nucleotide 26587 through nucleotide 27590; (7) a fourth exon, comprising a portion of the DCAL ORF, from nucleotide 27591 through nucleotide 27691; (8) a fourth intron from nucleotide 27692 through nucleotide 28125; (9) a fifth exon, comprising a portion of the DCAL ORF, from nucleotide 28126 through nucleotide 28228; (10) a fifth intron from nucleotide 28229 through nucleotide 29993; (11) a sixth exon, comprising a portion of the DCAL ORF, from nucleotide 29994 through nucleotide 30088; (12) a sixth intron from nucleotide 30089 through nucleotide 30655; (13) a seventh exon, comprising a portion of the DCAL ORF, from nucleotide 30656 through nucleotide 30762 (14) a seventh intron from nucleotide 30763 through nucleotide 31574; (15) an eighth exon, comprising a portion of the DCAL ORF, from nucleotide 31575 through nucleotide 31691; (16) an eighth intron from nucleotide 31692 through nucleotide 31897; (17) a ninth exon, comprising a portion of the DCAL ORF, from nucleotide 31898 through nucleotide 31961; (18) a ninth intron from nucleotide 31962 through nucleotide 32725; (19) a tenth exon, comprising a portion of the DCAL ORF, from nucleotide 32726 through nucleotide 32814 (20) a tenth intron from nucleotide 32815 through nucleotide 33176; (21) an eleventh exon, comprising a portion of the DCAL ORF, from nucleotide 33177 through nucleotide 33247; (22) an eleventh intron from nucleotide 33248 through nucleotide 33603; (23) a twelfth exon, comprising a portion of the DCAL ORF, from nucleotide 33604 through nucleotide 33698; (24) a twelfth intron from nucleotide 33699 through nucleotide 33782; (25) a thirteenth exon, comprising a portion of the DCAL ORF, from nucleotide 33783 through nucleotide 33881; (26) a thirteenth intron from nucleotide 33882 through nucleotide 34281; (27) a fourteenth exon, comprising a portion of the DCAL ORF, from nucleotide 34282 through nucleotide 34350; (28) a fourteenth intron from nucleotide 34351 through nucleotide 34778; (29) a fifteenth exon, comprising a portion of the DCAL ORF, from nucleotide 34779 through nucleotide 34857; (30) a fifteenth intron from nucleotide 34858 through nucleotide 35961; (31) a sixteenth exon, comprising a portion of the DCAL ORF, from nucleotide 35962 through nucleotide 36082; (32) a sixteenth intron from nucleotide 36083 through nucleotide 36904; (33) a seventeenth exon, comprising an end portion of the DCAL ORF, from nucleotide 36905 through nucleotide 36908 and a 3′ untranslated region from nucleotide 36909 to nucleotide 37620.

In another embodiment, the polynucleotide comprises the entire sequence of nucleotides set forth in SEQ ID NO: 5, which corresponds to a 33000-bp mouse genomic sequence for DCAL. This sequence defines: (1) a first exon from nucleotide 777 through nucleotide 1133, which exon comprises a 5′ untranslated region from nucleotide 777 through nucleotide 1013 and a portion of the DCAL open reading frame (ORF) from nucleotide 1014 through nucleotide 1133; (2) a first intron from nucleotide 1134 through nucleotide 19464; (3) a second exon, comprising a portion of the DCAL ORF, from nucleotide 19465 through nucleotide 19576; (4) a second intron from nucleotide 19577 through nucleotide 22142; (5) a third exon, comprising a portion of the DCAL ORF, from nucleotide 22143 through nucleotide 22286; (6) a third intron from nucleotide 22287 through nucleotide 23188; (7) a fourth exon, comprising a portion of the DCAL ORF, from nucleotide 23189 through nucleotide 23289; (8) a fourth intron from nucleotide 23290 through nucleotide 23758; (9) a fifth exon, comprising a portion of the DCAL ORF, from nucleotide 23759 through nucleotide 23861; (10) a fifth intron from nucleotide 23862 through nucleotide 25161; (11) a sixth exon, comprising a portion of the DCAL ORF, from nucleotide 25162 through nucleotide 26256; (12) a sixth intron from nucleotide 26257 through nucleotide 26053; (13) a seventh exon, comprising a portion of the DCAL ORF, from nucleotide 26054 through nucleotide 26160 (14) a seventh intron from nucleotide 26161 through nucleotide 26978; (15) an eighth exon, comprising a portion of the DCAL ORF, from nucleotide 26979 through nucleotide 27095; (16) an eighth intron from nucleotide 27096 through nucleotide 27343; (17) a ninth exon, comprising a portion of the DCAL ORF, from nucleotide 27344 through nucleotide 27407; (18) a ninth intron from nucleotide 27408 through nucleotide 28117; (19) a tenth exon, comprising a portion of the DCAL ORF, from nucleotide 28118 through nucleotide 28206 (20) a tenth intron from nucleotide 28207 through nucleotide 28561; (21) an eleventh exon, comprising a portion of the DCAL ORF, from nucleotide 28562 through nucleotide 28632; (22) an eleventh intron from nucleotide 28633 through nucleotide 28954; (23) a twelfth exon, comprising a portion of the DCAL ORF, from nucleotide 28955 through nucleotide 29049; (24) a twelfth intron from nucleotide 29050 through nucleotide 29133; (25) a thirteenth exon, comprising a portion of the DCAL ORF, from nucleotide 29134 through nucleotide 29232; (26) a thirteenth intron from nucleotide 29233 through nucleotide 29590; (27) a fourteenth exon, comprising a portion of the DCAL ORF, from nucleotide 29591 through nucleotide 29659; (28) a fourteenth intron from nucleotide 29660 through nucleotide 30078; (29) a fifteenth exon, comprising a portion of the DCAL ORF, from nucleotide 30079 through nucleotide 30157; (30) a fifteenth intron from nucleotide 30158 through nucleotide 31662; (31) a sixteenth exon, comprising a portion of the DCAL ORF, from nucleotide 31663 through nucleotide 31783; (32) a sixteenth intron from nucleotide 31784 through nucleotide 32187; (33) a seventeenth exon from nucleotide 32188 through nucleotide 32795, comprising an end portion of the DCAL ORF, from nucleotide 32188 through nucleotide 32191 and a 3′ untranslated region from nucleotide 32192 to nucleotide 32795.

Both the human and mouse DCAL genes have 17 exons with identical exon-intron junction organization. The ORFs of the human and murine DCAL genes each encode a polypeptide comprising a hydrophilic domain (encoded by nucleotides 300-401, 24623-24374 and 26443-26528 of SEQ ID NO: 1 or by nucleotides 1014-1133, 19465-19576 and 22143-22228 of SEQ ID NO: 5) and an AHCY-like domain (encoded by nucleotides 26529-26586, 27591-27691, 28126-28228, 29994-30088, 30656-30762, 31575-61691, 31898-31961, 32726-32814, 33177-33247, 33604-33698, 33783,-33881, 34282-34350, 34779-34857, 35962-36082 and 36905-36908 of SEQ ID NO: 1, or by nucleotides 26529-22286, 23189-23289, 23759-23861, 25162-26256, 26054-26160, 26979-27095, 27344-27407, 28228-28206, 28562-28632, 28955-29049, 29134-29232, 29591-29659, 30079-30157, 31663-31783 and 32188-32191 of SEQ ID NO: 5). The ORF also comprises a NAD⁺ binding motif (GXGXXG) at amino acid positions 318 to 323 (encoded by nucleotides 31950-31961 and 32726-32731 of SEQ ID NO: 1 and by nucleotides 27396-27407 and 28118-28123 of SEQ ID NO: 5) and the acidic amino acid 18 positions downstream of the motif (Glu³⁴¹, encoded by nucleotides 32783-32785 of SEQ ID NO: 1 or by nucleotides 28175-28177 of SEQ ID NO: 5) as well as Lys⁵²⁴ (encoded by nucleotides 36066-36068 of SEQ ID NO: 1 or by nucleotides 31767-31769 of SEQ ID NO: 5) and Tyr⁵²⁸ (encoded by nucleotides 36078-36080 of SEQ ID NO: 1 or by nucleotides 31779-31781 of SEQ ID NO: 5). Residues of DCAL predicted to bind a nucleotide or nucleotide-containing molecule include, but are not restricted to, His¹⁵³, Cys¹⁶⁶, Ser¹⁸¹, Asp²²⁹, Glu²⁵⁴, Ser²⁵⁵, Asn²⁷⁹, Lys²⁸⁴, Asp²⁸⁸, Asn²⁸⁹, His³⁸⁴, His³⁹⁹ (encoded respectively by nucleotides 27671-27673, 28144-28146, 28189-28191, 30666-30668, 30740-30742, 30743-30745, 31627-31629, 31642-31644, 31654-31656, 31657-31659, 33630-33632 and 33675-33677 of SEQ ID NO: 1 or encoded respectively by nucleotides 23269-23271, 23777-23779, 23822-23824, 26063-26065, 26138-26140, 26141-26143, 27025-27027, 27046-27048, 27058-27060, 27061-27063, 28981-28983 and 29026-29028 of SEQ ID NO: 5).

The invention also provides DCAL polynucleotides corresponding to full-length DCAL cDNA. Thus, in one embodiment, the polynucleotide comprises the sequence set forth in SEQ ID NO: 3, which corresponds to the full-length human DCAL cDNA. In an alternate embodiment, the polynucleotide comprises the sequence set forth in SEQ ID NO: 7, which corresponds to the full-length mouse DCAL cDNA.

A BLAST 2 comparison, with standard defaults, of human and mouse cDNA sequences (SEQ ID NO: 3 and SEQ ID NO: 7) reveals that the human DCAL cDNA sequence displays 89% sequence identity over its entire length to the murine DCAL cDNA. The conservation is even more pronounced at the amino acid level with the human DCAL polypeptide displaying greater than 99% sequence identity over its entire length to the murine counterpart. In view of this high interspecies conservation, it is predicted that other mammalian DCAL genomic sequences and cDNA molecules will have a very similar structure and sequence relative to the human and murine DCAL genes and cDNA molecules of the invention. Thus, the present specification provides sufficient information for a skilled person in the art to isolate or otherwise produce polynucleotide sequences, including genomic and cDNA sequences from other mammals, which could be used to produce genetically modified non-human animals, as described infra.

The invention encompasses isolated or substantially purified nucleic acid compositions. An “isolated” or “purified” nucleic acid molecule, or biologically active portion thereof, is substantially or essentially free from components that normally accompany or interact with the nucleic acid molecule as found in its naturally occurring environment. Thus, an isolated or purified polynucleotide is substantially free of other cellular material, or culture medium when produced by recombinant techniques, or substantially free of chemical precursors or other chemicals when chemically synthesized. Suitably, an “isolated” polynucleotide is free of sequences (especially protein encoding sequences) that naturally flank the polynucleotide (i.e., sequences located at the 5′ and 3′ ends of the polynucleotide) in the genomic DNA of the organism from which the polynucleotide was derived. For example, in various embodiments, the isolated polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide was derived.

The present invention also encompasses portions of the disclosed nucleotide sequences. Portions of a nucleotide sequence may encode polypeptide portions or segments that retain the biological activity of the native polypeptide. Alternatively, portions of a nucleotide sequence that are useful as hybridization probes generally do not encode amino acid sequences retaining such biological activity. Thus, portions of a nucleotide sequence may range from at least about 15, 16, 17, 18, 19, 20, 21, 22, 23, 24, 25, 26, 27, 28, 29, 30, 40, 50, 60, 80, 90, 100 nucleotides, or almost up to the full-length nucleotide sequence encoding the polypeptides of the invention.

A portion of a DCAL nucleotide sequence that encodes a biologically active portion of a DCAL polypeptide of the invention will encode at least about 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 300, 400 or 500 contiguous amino acid residues, or almost up to the total number of amino acids present in a full-length DCAL polypeptide of the invention (for example, 500 amino acid residues for SEQ ID NO: 4 or 6). Portions of a DCAL nucleotide sequence that are useful as hybridization probes or PCR primers generally need not encode a biologically active portion of a DCAL polypeptide.

Thus, a portion of a DCAL nucleotide sequence may encode a biologically active portion of a DCAL polypeptide, or it may be a fragment or segment that can be used as a hybridization probe or PCR primer using standard methods known in the art. Nucleic acid molecules that are portions of a DCAL nucleotide sequence comprise at least about 15, 16, 17, 18, 19, 20, 25, 30, 50, 75, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 200, 2500, 3000, 3500, 4000, 4500, 5000, 6000, 7000, 8000, 9000, 10000, 15000, 20000, 25000, 30000, 35000 or 40000 nucleotides, or almost up to the number of nucleotides present in a full-length DCAL nucleotide sequence disclosed herein (for example, 44000 or 32990 nucleotides for SEQ ID NO: 1 or 5, respectively).

2. DCAL Polynucleotides Variants

In general, polynucleotide variants according to the invention comprise regions that show at least 60%, more suitably at least 70%, preferably at least 75%, more preferably at least 80%, more preferably at least 85%, even more preferably at least 90% and still even more preferably at least 91%, 92%, 93%, 94%, 95%, 96%, 97%, 98% or 99% sequence identity over a reference polynucleotide sequence of identical size (“comparison window”) or when compared to an aligned sequence in which the alignment is performed by a computer homology program known in the art.

The DCAL gene sequence, including flanking promoter regions and coding regions, may be modified or mutated in various ways known in the art to generate targeted changes in promoter strength, sequence of the encoded protein, etc. The sequence changes may be substitutions, insertions or deletions. Deletions may include large changes, such as deletions of a domain or exon. Other modifications of interest include epitope tagging, e.g. with the FLAG system, HA, etc. For studies of subcellular localization, fusion proteins with green fluorescent proteins (GFP) may be used. Such mutated genes may be used to study structure-function relationships of DCAL polypeptides, or to alter properties of the proteins that affect their function or regulation.

For example, a polynucleotide according to any one of SEQ ID NO: 1, 3, 5 or 7 can be mutated using random mutagenesis (e.g., transposon mutagenesis), oligonucleotide-mediated (or site-directed) mutagenesis, PCR mutagenesis and cassette mutagenesis of an earlier prepared variant or non-variant version of an isolated natural promoter according to the invention.

Oligonucleotide-mediated mutagenesis is a preferred method for preparing nucleotide substitution variants of a polynucleotide of the invention. This technique is well known in the art as, for example, described by Adelman et al. (1983, DNA 2:183). Briefly, a polynucleotide according to any one of SEQ ID NO: 1, 3, 5 or 7 is altered by hybridizing an oligonucleotide encoding the desired mutation to a template DNA, wherein the template is the single-stranded form of a plasmid or bacteriophage containing the unaltered or parent DNA sequence. After hybridization, a DNA polymerase is used to synthesize an entire second complementary strand of the template that will thus incorporate the oligonucleotide primer, and will code for the selected alteration in the parent DNA sequence.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule.

The DNA template can be generated by those vectors that are either derived from bacteriophage M13 vectors, or those vectors that contain a single-stranded phage origin of replication as described by Viera et al. (1987, Methods Enzymol. 153:3). Thus, the DNA that is to be mutated may be inserted into one of the vectors to generate single-stranded template. Production of single-stranded template is described, for example, in Sections 4.21-4.41 of Sambrook et al. (1989, supra).

Alternatively, the single-stranded template may be generated by denaturing double-stranded plasmid (or other DNA) using standard techniques.

For alteration of the native DNA sequence, the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of the polypeptide or fragment under test, and the other strand (the original template) encodes the native unaltered sequence of the polypeptide or fragment under test. This heteroduplex molecule is then transformed into a suitable host cell, usually a prokaryote such as E. coli. After the cells are grown, they are plated onto agarose plates and screened using the oligonucleotide primer having a detectable label to identify the bacterial colonies having the mutated DNA. The resultant mutated DNA fragments are then cloned into suitable expression hosts such as E. coli using conventional technology and clones that retain the desired antigenic activity are detected. Where the clones have been derived using random mutagenesis techniques, positive clones would have to be sequenced in order to detect the mutation.

Alternatively, linker-scanning mutagenesis of DNA may be used to introduce clusters of point mutations throughout a sequence of interest that has been cloned into a plasmid vector. For example, reference may be made to Ausubel et al., supra, (in particular, Chapter 8.4) which describes a first protocol that uses complementary oligonucleotides and requires a unique restriction site adjacent to the region that is to be mutagenised. A nested series of deletion mutations is first generated in the region. A pair of complementary oligonucleotides is synthesized to fill in the gap in the sequence of interest between the linker at the deletion endpoint and the nearby restriction site. The linker sequence actually provides the desired clusters of point mutations as it is moved or “scanned” across the region by its position at the varied endpoints of the deletion mutation series. An alternate protocol is also described by Ausubel et al., supra, which makes use of site directed mutagenesis procedures to introduce small clusters of point mutations throughout the target region. Briefly, mutations are introduced into a sequence by annealing a synthetic oligonucleotide containing one or more mismatches to the sequence of interest cloned into a single-stranded M13 vector. This template is grown in an E. coli dut⁻ ung⁻ strain, which allows the incorporation of uracil into the template strand. The oligonucleotide is annealed to the template and extended with T4 DNA polymerase to create a double-stranded heteroduplex. Finally, the heteroduplex is introduced into a wild-type E. coli strain, which will prevent replication of the template strand due to the presence of apurinic sites (generated where uracil is incorporated), thereby resulting in plaques containing only mutated DNA.

Region-specific mutagenesis and directed mutagenesis using PCR may also be employed to construct polynucleotide variants according to the invention. In this regard, reference may be made, for example, to Ausubel et al., supra, in particular Chapters 8.2A and 8.5.

Alternatively, suitable polynucleotide sequence variants of the invention may be prepared according to the following procedure:
(a) creating primers which are optionally degenerate wherein each comprises a portion of a reference polynucleotide including, but not restricted to, a DCAL gene sequence as for example set forth in SEQ ID NO: 1 or 5, or a sequence encoding a reference polypeptide or fragment of the invention, or a complement of the sequence, the polypeptide preferably encoding the sequence set forth in any one of SEQ ID NO: 2 or 8;
(b) obtaining a nucleic acid extract from an organism, which is preferably an animal, and more preferably a mammal, including, but not restricted to primates, rodents, canines, felines, bovines, ovines, equines, and the like; and
(c) using the primers to amplify, via nucleic acid amplification techniques, at least one amplification product from the nucleic acid extract, wherein the amplification product corresponds to a polynucleotide variant.

Suitable nucleic acid amplification techniques are well known to the skilled addressee, and include polymerase chain reaction (PCR) as for example described in Ausubel et al. (supra); strand displacement amplification (SDA) as for example described in U.S. Pat. No 5,422,252; rolling circle replication (RCR) as for example described in Liu et al., (1996, J. Am. Chem. Soc. 118:1587-1594 and International application WO 92/01813) and Lizardi et al., (International Application WO 97/19193); nucleic acid sequence-based amplification (NASBA) as for example described by Sooknanan et al., (1994, Biotechniques 17:1077-1080); and Q-β replicase amplification as for example described by Tyagi et al., (1996, Proc. Natl. Acad. Sci. USA 93: 5395-5400).

Preferably, the variant is obtained from an animal and more preferably from a mammal.

Typically, polynucleotide variants that are substantially complementary to a reference polynucleotide are identified by blotting techniques that include a step whereby nucleic acids are immobilized on a matrix (preferably a synthetic membrane such as nitrocellulose), followed by a hybridization step, and a detection step. Southern blotting is used to identify a complementary DNA sequence; northern blotting is used to identify a complementary RNA sequence. Dot blotting and slot blotting can be used to identify complementary DNA/DNA, DNA/RNA or RNA/RNA polynucleotide sequences. Such techniques are well known by those skilled in the art, and have been described in Ausubel et al. (1994-1998, supra) at pages 2.9.1 through 2.9.20.

According to such methods, Southern blotting involves separating DNA molecules according to size by gel electrophoresis, transferring the size-separated DNA to a synthetic membrane, and hybridizing the membrane-bound DNA to a complementary nucleotide sequence labeled radioactively, enzymatically or fluorochromatically. In dot blotting and slot blotting, DNA samples are directly applied to a synthetic membrane prior to hybridization as above.

An alternative blotting step is used when identifying complementary polynucleotides in a cDNA or genomic DNA library, such as through the process of plaque or colony hybridization. A typical example of this procedure is described in Sambrook et al. (“Molecular Cloning. A Laboratory Manual”, Cold Spring Harbor Press, 1989) Chapters 8-12.

Typically, the following general procedure can be used to determine hybridization conditions. Polynucleotides are blotted/transferred to a synthetic membrane, as described above. A reference polynucleotide such as a polynucleotide of the invention is labeled as described above, and the ability of this labeled polynucleotide to hybridize with an immobilized polynucleotide is analyzed.

A skilled artisan will recognize that a number of factors influence hybridization. The specific activity of radioactively labeled polynucleotide sequence should typically be greater than or equal to about 10⁸ dpm/mg to provide a detectable signal. A radiolabelled nucleotide sequence of specific activity 10⁸ to 10⁹ dpm/mg can detect approximately 0.5 pg of DNA. It is well known in the art that sufficient DNA must be immobilized on the membrane to permit detection. It is desirable to have excess immobilized DNA, usually 10 μg. Adding an inert polymer such as 10% (w/v) dextran sulfate (MW 500,000) or polyethylene glycol 6000 during hybridization can also increase the sensitivity of hybridization (see Ausubel supra at 2.10.10).

To achieve meaningful results from hybridization between a polynucleotide immobilized on a membrane and a labeled polynucleotide, a sufficient amount of the labeled polynucleotide must be hybridized to the immobilized polynucleotide following washing. Washing ensures that the labeled polynucleotide is hybridized only to the immobilized polynucleotide with a desired degree of complementarity to the labeled polynucleotide.

It will be understood that polynucleotide variants according to the invention will hybridize to a reference polynucleotide under at least low stringency conditions. Reference herein to low stringency conditions includes and encompasses from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Suitably, the polynucleotide variants hybridize to a reference polynucleotide under at least medium stringency conditions. Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C.

Preferably, the polynucleotide variants hybridize to a reference polynucleotide under high stringency conditions. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C.

Other stringent conditions are well known in the art. A skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula:
Tₘ=81.5+16.6 (log₁₀ M)+0.41 (% G+C)−0.63 (% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex.

The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In a preferred hybridization procedure, a membrane (e.g., a nitrocellulose membrane or a nylon membrane) containing immobilized DNA is hybridized overnight at 42° C. in a hybridization buffer (50% deionised formamide, 5×SSC, 5×Denhardt's solution (0.1% ficoll, 0.1% polyvinylpyrollidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.).

Methods for detecting a labeled polynucleotide hybridized to an immobilized polynucleotide are well known to practitioners in the art. Such methods include autoradiography, phosphorimaging, and chemiluminescent, fluorescent and colorimetric detection.

C. Genetically Modified Animals of the Invention

The invention also provides genetically modified, non-human animals having an altered DCAL gene. Alterations to the DCAL gene include, but are not restricted to, deletions or other loss of function mutations, introduction of an exogenous gene having a nucleotide sequence with targeted or random mutations, introduction of an exogenous gene from another species, or a combination thereof. The genetically modified animal may be either homozygous or heterozygous for the alteration.

Useful sequences for producing the genetically modified animals of the invention include, but are not restricted to, open reading frames encoding specific polypeptides or domains, introns, and adjacent 5′ and 3′ non-coding nucleotide sequences involved in the regulation of expression, up to about 1 kb beyond the coding region, but possibly further in either direction. The DNA sequences encoding DCAL may be cDNA (e.g., SEQ ID NO: 3 or 7) or genomic DNA (e.g., SEQ ID NO: 1 or 5) or a portion thereof. A genomic sequence of interest comprises the nucleic acid present between the initiation codon and the stop codon, as defined in the listed sequences, including all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kb or smaller; and substantially free of flanking chromosomal sequence. The sequence of this 5′ region, and further 5′ upstream sequences and 3′ downstream sequences, may be utilized for promoter elements, including enhancer binding sites, that provide for expression in cells where DCAL is expressed. The cell specific expression is useful for determining the pattern of expression, and for providing promoters that mimic the native pattern of expression. Naturally occurring polymorphisms in the promoter region are useful for determining natural variations in expression, particularly those that may be associated with disease. Alternatively, mutations may be introduced into the promoter region to determine the effect of altering expression in experimentally defined systems. Methods for the identification of specific DNA motifs involved in the binding of transcriptional factors are known in the art, e.g. sequence similarity to known binding motifs, gel retardation studies, etc. For examples, reference may be made to Blackwell et al. (1995, Mol Med 1: 194-205), Mortlock et al. (1996, Genome Res. 6: 327-33), and Joulin and Richard-Foy (1995, Eur J Biochem 232: 620-626). Further, there is recent evidence that expression of certain mRNA species can be regulated at the translational level so that protein expression is restricted to particular cells types. The key features of these mRNA species are multiple translational initiation sites in the 5′ region of the coding sequence and a long 3′ untranslated region that controls mRNA translation in part. Recent Northern blotting data suggest that the full-length DCAL mRNA is approximately 4.4 kb and that its 3′ untranslated region forms a large portion of the message. Nucleotide sequence analysis has also revealed the presence of multiple putative translation initiation sites in the 5′ region of the DCAL mRNA. Accordingly, it is proposed that DCAL has all the features required for such post transcriptional regulation and that DCAL function can be altered by disrupting or inactivating the 3′ and/or 5′ untranslated regions found in the DCAL mRNA.

The regulatory sequences may be used to identify cis acting sequences required for transcriptional or translational regulation of DCAL expression, especially in different cells or stages of development or differentiation, and to identify cis acting sequences and trans acting factors that regulate or mediate expression. Such transcription or translational control regions may be operably linked to a DCAL gene in order to promote expression of wild type or altered DCAL or other proteins of interest in cultured cells, or in embryonic, fetal or adult tissues, and for gene therapy.

The polynucleotides used in the subject invention may encode all or a part of the DCAL polypeptides or domains thereof as appropriate. Fragments of the DNA sequence may be obtained by chemically synthesizing oligonucleotides in accordance with conventional methods, by restriction enzyme digestion, by PCR amplification, etc. For the most part, DNA fragments will be of at least 15 nucleotides, usually at least 18 nucleotides, more usually at least about 50 nucleotides. Such small DNA fragments are useful as primers for PCR, hybridization screening, etc. Larger DNA fragments, i.e. greater than 100 nucleotides are useful for production of the encoded polypeptide. For use in amplification reactions, such as PCR, a pair of primers will be used.

The genetically modified animals of the present invention typically, but not exclusively, comprise a foreign or exogenous polynucleotide sequence or transgene present as an extrachromosomal element or stably integrated in all or a portion of its cells, especially in germ cells. Unless otherwise indicated, it will be assumed that a genetically modified animal comprises stable changes to the germline sequence. During the initial construction of the animal, “chimeras” or “chimeric animals” are generated, in which only a subset of cells have the altered genome. Chimeras are primarily used for breeding purposes in order to generate the desired genetically modified animal. Animals having a heterozygous alteration are generated by breeding of chimeras. Male and female heterozygotes are typically bred to generate homozygous animals.

Genetically modified animals fall into two groups, colloquially termed “knockouts” and “knockins”. In the present invention, knockouts have a partial or complete loss of function in one or both alleles of the endogenous DCAL gene. Knockins have an introduced transgene (i.e., foreign gene) with altered genetic sequence and function from the endogenous gene. Increased (including ectopic) or decreased expression may be achieved by introduction of an additional copy of the target gene, or by operatively inserting a regulatory sequence that provides for enhanced expression of an endogenous copy of the target gene. These changes may be constitutive or conditional, i.e. dependent on the presence of an activator or repressor. The foreign gene is usually either from a different species than the animal host, or is otherwise altered in its coding or non-coding sequence. The introduced gene may be a wild-type gene, naturally occurring polymorphism, or a genetically manipulated sequence, for example having deletions, substitutions or insertions in the coding or non-coding regions. The introduced sequence may encode a DCAL polypeptide, or may utilise the DCAL promoter operably linked to a reporter gene. Where the introduced gene is a coding sequence, it is usually operably linked to a promoter, which may be constitutive or inducible, and other regulatory sequences required for expression in the host animal. A knockin and a knockout may be combined, such that the naturally occurring gene is disabled, and an altered form introduced.

Preferably, a genetically modified animal of the invention has a partial or complete loss of function in one or both alleles of the endogenous DCAL gene and thus falls into the “knockout” group of genetically modified animals. A knockout may be achieved by a variety of mechanisms, including introduction of a disruption of the coding sequence, e.g. insertion of one or more stop codons, insertion of a DNA fragment, etc., deletion of coding sequence, substitution of stop codons for coding sequence, etc. In some cases the foreign transgene sequences are ultimately deleted from the genome, leaving a net change to the native sequence. Different approaches may be used to achieve the “knockout”. A chromosomal deletion of all or part of the native DCAL may be induced, including deletions of the non-coding regions, particularly the promoter region, 3′ regulatory sequences, enhancers, or deletion of a gene that activates expression of DCAL. A functional knockout may also be achieved by the introduction of an anti-sense construct that blocks expression of the native DCAL genes (for example, see Li and Cohen, 1996, Cell 85: 319-329). “Knockouts” also include conditional knock-outs in which DCAL is inactivated or loses function in a tissue-specific or a temporal-specific pattern, for example where alteration of the DCAL gene occurs upon exposure of the animal to a substance that promotes DCAL alteration, introduction of an enzyme that promotes recombination at the DCAL gene site (e.g. Cre in the Cre-lox system), or other method for directing the DCAL alteration postnatally. In illustrative examples of this type, a modulator gene is supplied to the genetically modified animal, which comprises a nucleic acid sequence that encodes a site-specific recombinase protein that typically recognizes target sites located within or adjacent to the DCAL gene, whereby excision of a nucleotide sequence between the target sites by the recombinase results in a reduction in the level or functional activity of a DCAL gene expression product. Illustrative site-specific recombinases include, but are not limited to, Cre, FLP-wild type (wt), FLP-L or FLPe. Recombination may be effected by any art-known method, e.g., the method of Doetschman et al. (1987, Nature 330:576-578); the method of Thomas et al. (1986, Cell 44:419-428); the Cre-loxP recombination system (Sternberg and Hamilton, 1981, J. Mol. Biol. 150:467-486; Lakso et al., 1992, Proc. Natl. Acad. Sci. USA 89:6232-6236); the FLP recombinase system of Saccharomyces cerevisiae (O'Gorman et al., 1991, Science 251:1351-1355; Lyznik et al., 1996, Nucleic Acids Res. 24(19):3784-3789); the Cre-loxP-tetracycline control switch (Gossen and Bujard, 1992, Proc. Natl. Acad. Sci. USA 89:5547-51); and ligand-regulated recombinase system (Kellendonk et al., 1999, J. Mol. Biol. 285:175-82). Desirably, the recombinase is highly active, e.g., the Cre-loxP or the FLPe system, and has enhanced thermostability (Rodrguez et al., 2000, Nature Genetics 25:139-40). In specific embodiments, the modulator gene encodes a Cre recombinase or an FLP recombinase and at least a portion of the DCAL gene (optionally including its regulatory sequences) is flanked by either loxP target sites, which are specifically recognized by the Cre recombinase, or FRT target sites, which are specifically recognized by the FLP recombinase.

Several other recombination systems are also suitable for use in the present invention. These include, for example, the Gin recombinase of phage Mu (Crisona et al., 1994, J. Mol. Biol. 243(3):437-457), the Pin recombinase of E. coli (see, e.g., Kutsukake et al., 1985, Gene 34(2-3):343-350), the PinB, PinD and PinF from Shigella (Tominaga et al., 1991, J. Bacteriol. 173(13):4079-4087), the R/RS system of the pSR1 plasmid (Araki et al., 1992, J. Mol. Biol. 225(1):25-37) and the cin, hin and β-recombinases. Other recombination systems relevant to this invention described herein are those from Kluyveromyces species, phages, and integrating viruses (e.g., the SSV1-encoded integrase).

In certain embodiments, the recombinase system can be linked to a second inducible or repressible transcriptional regulation system. For example, a cell-specific Cre-loxP mediated recombination system (Gossen and Bujard, 1992, Proc. Natl. Acad. Sci. USA 89:5547-51) can be linked to a cell-specific tetracycline-dependent time switch (see, e.g., Ewald et al., 1996, Science 273:1384-1386; Furth et al., 1994, Proc. Natl. Acad. Sci. U.S.A. 91:9302-9306; St-Onge et al., 1996, Nucleic Acids Res. 24(19): 3875-7387). In an illustrative example, an altered cre gene with enhanced expression in mammalian cells is used (Gorski and Jones, 1999, Nucleic Acids Res. 27(9): 2059-2061).

In another illustrative example, the ligand-regulated recombinase system of Kellendonk et al. (1999, J. Mol. Biol. 285: 175-182) can be used. In this system, the ligand-binding domain (LBD) of a receptor, is fused to the Cre recombinase to increase specificity of the recombinase. In this way, the activity of the recombinase is controlled by the presence of the ligand in the cell for the nuclear receptor. The LBD suitably comprises a derivative of part or all of a nuclear receptor, where the part includes the ligand-binding portion of a nuclear receptor. The nuclear receptor may be endogenous to the host or may be derived from another species. The nuclear receptor derivative thereof may be selected from steroid-hormone dependent receptors, which include estrogen, androgen, adrenal glucocorticoid, aldosterone and progesterone receptors; nuclear hormone receptors, which include vitamin D, retinoid, thyroid hormone receptors; and orphan nuclear receptors, which include peroxisome proliferator activated receptors and lipid receptors such as, but not limited to, COUP-TFI/II and SF-1. Suitably, the ligand-binding portion of the nuclear receptor is a portion or derivative of a steroid-hormone dependent receptor and is desirably a derivative of the estrogen receptor LBD. Advantageously, the estrogen receptor LBD derivative exhibits reduced or absent affinity for endogenous estrogen and estrogen-related hormones, with reference to a normal, reference range of binding affinity. In certain embodiments of this type, the LBD of the estrogen receptor derivative exhibits affinity for non-endogenous estrogen hormone analogues such as Tamoxifen and analogues thereof. The ligand-binding domain may be fused to the N′ or C′ terminus of the recombinase protein. In certain examples, the estrogen-receptor binding domain is fused to the C-terminus of the Cre recombinase protein.

In some embodiments, the DCAL gene is conditionally expressed in a tissue-specific manner, e.g., in hematopoietic cells such as but not limited to mast cells, basophils, eosinophils, neutrophils, lymphocytes (e.g., T cells and B cells), macrophages, dendritic cells and natural killer cells. In specific embodiments, the hematopoietic cells are dendritic cells.

In specific embodiments, the partial or complete loss of function in one or both alleles of the DCAL gene is effected by disruption of that gene. Accordingly, the genetically modified animal preferably comprises a disruption in at least one allele of the endogenous DCAL gene or a genetically modified DCAL gene that is capable of disruption. In accordance with the present invention, disruption of the DCAL gene suitably results in an inability of the animal to produce a corresponding functional expression product or detectable levels of the expression product. Accordingly, a disruption in the endogenous DCAL gene may result in a reduced level and/or functional activity of DCAL or in an inability of the animal to produce a functional DCAL or detectable levels of DCAL relative to a corresponding animal without the disruption.

A disruption typically comprises an insertion of a nucleic acid sequence into one region of the native genomic sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a selectable marker gene which is inserted into a targeting nucleic acid sequence that is complementary to a genomic sequence (promoter and/or coding region) to be disrupted. Useful genomic sequences to be disrupted include, but are not restricted to, DCAL open reading frames encoding polypeptides or domains, introns, and adjacent 5′ and 3′ non-coding nucleotide sequences involved in regulation of gene expression. Accordingly, a targeting sequence may comprise some or part of the nucleic acid present between the initiation codon and the stop codon, as defined in the listed sequences, including some or all of the introns that are normally present in a native chromosome. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. When the nucleic acid construct is then transfected into a cell, the construct will integrate into the genomic DNA. Thus, many progeny of the cell will no longer express the gene at least in some cells, or will express it at a decreased level, as the genomic sequence is now disrupted by the selection marker.

The disruption in the endogenous DCAL gene preferably comprises a deletion of a DCAL nucleotide sequence encoding at least a portion of a DCAL polypeptide. In one embodiment, the disruption comprises a deletion of DCAL gene nucleotide sequences encoding a region of the AHCY-like domain of the animal DCAL. In another embodiment, the disruption comprises a deletion of DCAL gene nucleotide sequences encoding a region of the hydrophilic domain of the animal DCAL. In a preferred embodiment, the disruption comprises a deletion of at least a portion of exon 2 and/or exon 3 of the DCAL gene. In still other embodiments, the disruption comprises a deletion of exon 3 to 6 of the DCAL gene.

In some embodiments, an individual disruption reduces, abrogates or otherwise impairs the expression of a DCAL gene and in this regard, the disruption may reside in the deletion of at least a portion of the transcriptional and/or translational regulatory sequences associated with the DCAL gene.

Specific examples of the genetically modified animals of the present invention include those containing:
(a) a substantially complete loss of function in a single allele of the endogenous DCAL gene (i.e., DCAL^{+/-});
(b) a substantially complete loss of function in both alleles of the endogenous DCAL gene (i.e., DCAL^{-/-}); or
(c) genetic or functional equivalents of (a) or (b).

Suitable genetic or functional equivalent animals include those containing anti-sense constructs comprising a sequence complementary to at least a portion of an endogenous DCAL gene which will block expression of a corresponding expression product to a level analogous to that in (a) or (b) above. It should be understood that any and all such equivalents are contemplated to fall within the scope of the present invention.

Non-human animals for genetic modification include, but are not restricted to, vertebrates, preferably mammals such as rodents, non-human primates, ovines, bovines, ruminants, lagomorphs, porcines, caprines, equines, canines, felines, aves, etc. In a preferred embodiment, the non-human animal is selected from the order Rodentia, which includes rodents i.e., placental mammals (class Euthria) which include the family Muridae (rats and mice). In a particularly preferred embodiment, the non-human animal is a mouse.

D. Targeting Constructs

The invention provides a targeting vector for producing a genetically modified non-human animal model for DCAL function, comprising a polynucleotide of the invention or biologically active fragment thereof or variant or derivative of these. Specific constructs or vectors of interest include, but are not limited to, anti-sense DCAL constructs comprising a sequence complementary to a polynucleotide, fragment, variant or derivative as herein described, which will block native DCAL expression, expression of dominant negative DCAL mutations, and over-expression of a DCAL gene.

A detectable marker, such as lacZ, or a selection marker, such as neo, may be introduced into the locus, where upregulation of expression will result in an easily detected change in phenotype. Vectors utilizing the DCAL promoter region, in combination with a reporter gene or with the coding region are also of interest.

A series of small deletions and/or substitutions may be made in the DCAL gene to determine the role of different exons in DNA binding, transcriptional regulation, etc. By providing expression of DCAL protein in cells in which it is otherwise not normally produced, one can induce changes in cell behavior.

In a preferred embodiment, the vector is used to disrupt the DCAL gene. A gene disruption resulting in partial or complete loss of function in one or both alleles of DCAL is suitably effected using a targeting construct or vector. Any polynucleotide sequence capable of disrupting an endogenous gene of interest (e.g., by introducing a premature stop codon, causing a frameshift mutation, disrupting proper splicing, etc.) may be employed in this regard. In a preferred embodiment, the vector, or an ancillary vector, comprises a positive selectable marker gene (e.g., hyg or neo). The disruption may reduce or prevent the expression of DCAL or may render the resulting DCAL polypeptide completely non-functional. Reduced levels of DCAL refer to a level of DCAL which is lower than that found in a wild-type animal. The level of DCAL produced in an animal of interest may be determined by a variety of methods including Western blot analysis of protein extracted from suitable cell types including, but not restricted to, kidney cells, lymphocytes or melanocytes. A lack of ability to produce functional DCAL includes within its scope the production of undetectable levels of functional DCAL (e.g., by Western blot analysis). In contrast, a functional DCAL is a molecule which retains the biological activity of the wild-type DCAL and which preferably is of the same molecular weight as the wild-type molecule.

Targeting vectors for homologous recombination will comprise at least a portion of the DCAL gene with the desired genetic modification, and will include regions of homology to the target locus. Those regions may be non-isogenic, but are preferably isogenic, to the target locus. Conveniently, markers for positive and negative selection are included. Methods for generating cells having targeted gene modifications through homologous recombination are known in the art. Various techniques for transfecting animal and particularly mammalian cells are described for example by Keown et al. (1990, Methods in Enzymology 185: 527-537).

In a preferred embodiment, the targeting vector includes polynucleotide sequences comprising a selectable marker gene flanked on either side by DCAL gene sequences. The targeting vector will generally contain gene sequences sufficient to permit the homologous recombination of the targeting vector into at least one allele of the endogenous gene resident in the chromosomes of the target or recipient cell (e.g., ES cells). In a preferred embodiment, the cell employed is an ES cell from a mammal within the order Rodentia and most preferably a mouse ES cell. Typically, the targeting vector will contain approximately 1 to 15 kb of DNA homologous to the endogenous DCAL gene (more than 15 kb or less than 5 kb of the endogenous DCAL gene sequences may be employed so long as the amount employed is sufficient to permit homologous recombination into the endogenous gene); this 1 to 15 kb of DNA is preferably divided on each side of the selectable marker gene.

The targeting construct may contain more than one selectable marker gene. The selectable marker is preferably a polynucleotide which encodes an enzymatic activity that confers resistance to an antibiotic or drug upon the cell in which the selectable marker is expressed. Selectable markers may be “positive”; positive selectable markers typically are dominant selectable markers, i.e., genes which encode an enzymatic activity which can be detected in any animal, preferably mammalian, cell or cell line (including ES cells). Examples of dominant selectable markers include the bacterial aminoglycoside 3′ phosphotransferase gene (also referred to as the neo gene) which confers resistance to the drug G418 in animal cells, the bacterial hygromycin G phosphotransferase (hyg) gene which confers resistance to the antibiotic hygromycin and the bacterial xanthine-guanine phosphoribosyl transferase gene (also referred to as the gpt gene) which confers the ability to grow in the presence of mycophenolic acid. Selectable markers may be ‘negative’; negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

When more than one selectable marker gene is employed, the targeting vector preferably contains a positive selectable marker (e.g., the neo gene) and a negative selectable marker (e.g., the Herpes simplex virus tk (HSV-tk) gene). The presence of the positive selectable marker permits the selection of recipient cells containing an integrated copy of the targeting vector whether this integration occurred at the target site or at a random site. The presence of the negative selectable marker permits the identification of recipient cells containing the targeting vector at the targeted site (i.e., which has integrated by virtue of homologous recombination into the target site); cells which survive when grown in medium which selects against the expression of the negative selectable marker do not contain a copy of the negative selectable marker.

Preferred targeting vectors of the present invention are of the “replacement-type”, wherein integration of a replacement-type vector results in the insertion of a selectable marker into the target gene. Replacement-type targeting vectors may be employed to disrupt a gene resulting in the generation of a null allele (i.e., an allele incapable of expressing a functional protein; null alleles may be generated by deleting a portion of the coding region, deleting the entire gene, introducing an insertion and/or a frameshift mutation, etc.) or may be used to introduce a modification (e.g., one or more point mutations) into a gene.

E. Methods of Producing the Genetically Modified Animals of the Invention

The genetically modified animals of the present invention are preferably generated by introduction of the targeting vectors into embryonic stem (ES) cells. ES cells are obtained by culturing pre-implantation embryos in vitro under appropriate conditions (Evans, et al., 1981, Nature 292: 154-156; Bradley, et al., 1984, Nature 309: 255-258; Gossler, et al., 1986, Proc. Natl. Acad. Sci. USA 83: 9065-9069; and Robertson, et al., 1986, Nature 322: 445-448). Transgenes can be efficiently introduced into the ES cells by DNA transfection using a variety of methods known to the art including electroporation, calcium phosphate co-precipitation, protoplast or spheroplast fusion, lipofection and DEAE-dextran-mediated transfection. Transgenes may also be introduced into ES cells by retrovirus-mediated transduction or by micro-injection. Cells are subsequently plated onto a feeder layer in an appropriate medium and those containing the transgene may be detected by employing a selective medium. Alternatively, PCR may be used to screen for ES cells which have integrated the transgene. After sufficient time for colonies to grow, they are picked and analyzed for the occurrence of homologous recombination or integration of the vector. This PCR technique obviates the need for growth of the transfected ES cells under appropriate selective conditions prior to transfer into the blastocoele of a non-human animal. Those colonies that are positive may then be used for embryo manipulation and blastocyst injection. Blastocysts are obtained from 4 to 6 week old superovulated females. The ES cells are trypsinized, and the modified cells are injected into the blastocoele of the blastocyst. After injection, the blastocysts are returned to each uterine horn of pseudopregnant females. Females are then allowed to go to term and the resulting litters screened for mutant cells having the vector. By providing for a different phenotype of the blastocyst and the ES cells, chimeric progeny can be readily detected. For a review, see Jaenisch (1988, Science 240: 1468-1474). The chimeric progeny are screened for the presence of the transgene and males and females having the transgene are mated to produce homozygous progeny. If the gene alterations cause lethality at some point in development, tissues or organs can be maintained as allogeneic or congenic grafts or transplants, or in in vitro culture.

Alternative methods for the generation of genetically modified animals are known to those skilled in the art. For example, embryonic cells at various developmental stages can be used to introduce transgenes for the production of genetically modified animals. Different methods are used depending on the stage of development of the embryonic cell. The zygote, particularly at the pronucleal stage (i.e., prior to fusion of the male and female pronuclei), is a preferred target for micro-injection. The use of zygotes as a target for gene transfer has a major advantage in that in most cases the injected DNA will be incorporated into the host genome before the first cleavage (Brinster, et al., 1985, Proc. Natl. Acad. Sci. USA 82: 4438-4442). As a consequence, all cells of the genetically modified non-human animal will carry the incorporated transgene. This will in general also be reflected in the efficient transmission of the transgene to offspring of the founder since 50% of the germ cells will harbour the transgene. U.S. Pat. No. 4,873,191 describes a method for the micro-injection of zygotes.

Retroviral infection can also be used to introduce transgenes into a non-human animal. The developing non-human embryo can be cultured in vitro to the blastocyst stage. During this time, the blastomeres can be targets for retroviral infection (Janenich, 1976, Proc. Natl. Acad. Sci. USA 73: 1260-1264). Retroviral infection is easily and efficiently obtained by culturing the blastomeres on a monolayer of virus-producing cells (Van der Putten, supra; Stewart, et al., 1987, EMBO J. 6: 383-388). Alternatively, infection can be performed at a later stage. Virus or virus-producing cells can be injected into the blastocoele (Jahner, D. et al., 1982, Nature 298: 623-628). It is also possible to introduce transgenes into the germline, albeit with low efficiency, by intrauterine retroviral infection of the midgestation embryo (Jahner, D. et al., 1982, supra). An additional means of using retroviruses or retroviral vectors to create genetically modified animals known to the art involves the micro-injection of retroviral particles or mitomycin C-treated cells producing retrovirus into the perivitelline space of fertilized eggs or early embryos (PCT International Application Publication No. WO 90/08832) and Haskell and Bowen, 1995, Mol. Reprod. Dev. 40: 386).

In selecting lines of an animal species to work the present invention, they may be selected for criteria such as embryo yield, pronuclear visibility in the embryos, reproductive fitness, color selection of genetically modified offspring or availability of ES cell clones. For example, if genetically modified mice are to be produced, lines such as C57BL/6 may be preferred.

The age of the animals that are used to obtain embryos and to serve as surrogate hosts is a function of the species used. When mice are used, for example, pre-puberal females are preferred as they yield more embryos and respond better to hormone injections. In this regard, administration of hormones or other chemical compounds may be necessary to prepare the female for egg production, mating and/or implantation of embryos.

Genetically modified offspring of a surrogate host may be screened for the presence of the transgene by any suitable method. Screening may be accomplished by Southern or northern analysis using a probe that is complementary to at least a portion of the transgene or by PCR using primers complementary to portions of the transgene. Western blot analysis using an antibody against the protein encoded by the transgene may be employed as an alternative or additional method for screening. Alternative or additional methods for evaluating the presence of the transgene include without limitation suitable biochemical assays such as enzyme and/or immunological assays, histological stains for particular markers or enzyme activities and the like.

Progeny of the genetically modified mammals may be obtained by mating the genetically modified animal with a suitable partner or by in vitro fertilization using eggs and/or sperm obtained from the genetically modified animal. Where in vitro fertilization is used, the fertilized embryo is implanted into a surrogate host or incubated in vitro or both. Where mating is used to produce genetically modified progeny, the genetically modified animal may be back-crossed to a parental line, otherwise inbred or cross-bred with animals possessing other desirable genetic characteristics. In a preferred embodiment, such other characteristics may include the partial or complete loss of function in at least one allele of a gene affecting an element of the immune system (e.g., nude, scid, nod). The progeny may be evaluated for the presence of the transgene using methods described above, or other appropriate methods.

Genetically modified animals comprising genetic alterations resulting in partial or complete loss of function in one or both alleles of DCAL find a number of uses. As discussed in more detail below, DCAL knockout mice provide a means for screening test compounds beneficial for modulating DCAL function. In addition, these animals provide a means for screening compounds for the treatment or prevention in patients of conditions associated with dendritic cell function or with neuronal function. In a particular preferred embodiment, these animals provide a means for screening compounds for therapeutic use in patients, which are useful inter alia in modulating an immune response such as but not limited preventing or reducing allergic responses, autoimmune diseases, graft versus host diseases or host versus graft diseases.

F. Drug Screening Assays

Through use of the subject genetically modified animals or cells derived therefrom, one can identify ligands or substrates that bind to, modulate, antagonize or agonize DCAL. Screening to determine drugs that lack effect on DCAL is also of interest. Areas of investigation are the development of immunotherapies, e.g., immunosuppressive treatments. Of particular interest are screening assays for agents that have a low toxicity for human cells. Thus, the invention contemplates a process for screening a candidate agent for the ability to specifically modulate DCAL function. The process comprises administering a candidate agent to a genetically modified non-human animal as broadly described above and to a corresponding wild-type non-human animal, which is preferably a species or strain of animal from which the genetically modified animal was derived. The individual immune responses of the genetically modified animal and of the wild-type animal are then compared. Any suitable immune response might be evaluated for the purposes of this comparison. For example, the wild-type and genetically modified animals can each be afflicted with an allergy (e.g., asthma, allergic rhinitis, systemic anaphylaxis) or an autoimmune disease (e.g., diabetes mellitus, rheumatoid arthritis) and that allergy or autoimmune disease monitored following administration of the candidate agent. Alternatively, the animals can receive an allogeneic graft and any host versus graft disease monitored following administration of the candidate agent. A candidate agent tests positive as a specific modulator of DCAL function if there is a substantial modulation of immune response in the wild-type animal but there is no substantial modulation of immune response in the genetically modified animal.

Candidate agents encompassed by the present invention includes agents that enhance or otherwise augment the immune response or that reduce, suppress or otherwise impair the immune response to one or more antigens, which may be self antigens (i.e., autologous) or foreign antigens (i.e., allogeneic or xenogeneic). Thus in one embodiment, an agent may test positive in the above method if the immune response in the wild-type animal is enhanced and there is no such enhancement in the genetically modified animal. In a preferred embodiment, however, the candidate agent reduces, suppresses or otherwise impairs the immune response in the wild-type animal but exhibits no such change in immune response in the genetically modified animal. To suppress an immune response (“immunosuppression ”), as is well-known in the art, means to decrease an animal's capacity to respond to foreign or disease-specific antigens (e.g., self antigens) i.e., those cells primed to attack such antigens are decreased in number, activity, and ability to detect and destroy the those antigens. Strength of immune response is measured by standard tests including: direct measurement of peripheral blood lymphocytes by means known to the art; natural killer cell cytotoxicity assays (see, e.g., Provinciali M. et al (1992, J. Immunol. Meth. 155: 19-24), cell proliferation assays (see, e.g., Vollenweider, I. and Groseurth, P. J. (1992, J. Immunol. Meth. 149: 133-135), immunoassays of immune cells and subsets (see, e.g., Loeffler, D. A., et al. (1992, Cytom. 13: 169-174); Rivoltini, L., et al. (1992, Can. Immunol. Immunother. 34: 241-251); or skin tests for cell-mediated immunity (see, e.g., Chang, A. E. et al (1993, Cancer Res. 53: 1043-1050). Any statistically significant reduction in strength of immune response as measured by the foregoing tests is considered “reduced or suppressed immune response” or “immunosuppression” as used herein. Suppressed immune response is also indicated by physical manifestations such as a reduction in, or absence of, fever and inflammation, and alleviation of symptoms of a disease or condition including, but not restricted to, host versus graft disease, autoimmune disease, allergies etc, and the like. Such physical manifestations also define “reduced or suppressed immune response” or “immunosuppression” as used herein.

Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Such agents include, but are not limited to, peptides such as, for example, soluble peptides, including, but not restricted to, members of random peptide libraries (see, e.g., Lam et al., 1991, Nature 354: 82-84; Houghten et al., 1991, Nature 354: 84-86), and combinatorial chemistry-derived molecular library peptides made of D- and/or L-configuration amino acids, phosphopeptides (including, but not limited, to members of random or partially degenerate, directed phosphopeptide libraries; see, e.g., Songyang et al., 1993, Cell 72: 767-778); antibodies (including, but not limited to, polyclonal, monoclonal, humanized, anti-idiotypic, chimeric or single chain antibodies, and Fab, F(ab)₂ and Fab expression library fragments, and epitope-binding fragments thereof).

Other compounds which can be screened in accordance with the invention include but are not limited to small organic molecules that are able to gain entry into an appropriate cell and affect the expression of a gene (e.g., by interacting with the regulatory region or transcription factors involved in gene expression); or such compounds that affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

A wide variety of assays may be used for monitoring the immune response of an animal, including in vitro methods of assessing immunity (assessing the ability of DC to elicit a cell-mediated or humoral response; e.g., to elicit a cytotoxic T cell response), determination of the localization of drugs after administration, labeled in vitro protein-protein binding assays, protein-DNA binding assays, electrophoretic mobility shift assays, immunoassays for protein binding, and the like. Depending on the particular assay, whole animals may be used, or cell derived therefrom. Cells may be freshly isolated from an animal, or may be immortalized in culture. Cells of particular interest include neural and brain tissue.

Generally, a plurality of assay mixtures are run in parallel with different agent concentrations to obtain a differential response to the various concentrations. Typically, one of these concentrations serves as a negative control, i.e. at zero concentration or below the level of detection.

In a preferred embodiment, the screening assay comprises an assay for assessing cell-mediated or humoral immunity. For example, suitable assays include, but are not limited to, assays that measure the number or activity of cells primed to attack an antigen of interest, assays that measure the number or activity of peripheral blood lymphocytes, natural killer cell cytotoxicity assays (see, e.g., Provinciali M. et al., 1992, J. Immunol. Meth. 155: 19-24), cell proliferation assays (see, e.g., Vollenweider, I. and Groseurth, P. J. 1992, J. Immunol. Meth. 149: 133-135), immunoassays of immune cells and subsets (see, e.g., Loeffler, D. A., et al., 1992, Cytom. 13: 169-174; Rivoltini, L., et al., 1992, Can. Immunol. Immunother. 34: 241-251); or skin tests for cell-mediated immunity (see, e.g., Chang, A. E. et al., 1993, Cancer Res. 53: 1043-1050). The cytotoxic activity of T lymphocytes, and in particular the ability of cytotoxic T lymphocytes to be induced by antigen-presenting DC, may be assessed by any suitable technique known to those of skill in the art. For example, a sample comprising T lymphocytes to be assayed for cytotoxic activity is obtained and the T lymphocytes are then exposed to DC, which have been caused to present antigen. After an appropriate period of time, which may be determined by assessing the cytotoxic activity of a control population of T lymphocytes which are known to be capable of being induced to become cytotoxic cells, the T lymphocytes to be assessed are tested for cytotoxic activity in a standard cytotoxic assay. Such assays may include, but are not limited to, the chromium release CTL assay known in the art. The method of assessing CTL activity is particularly useful for evaluating an individual's capacity to generate a cytotoxic response against cells expressing self antigens. Accordingly, this method is useful inter alia for evaluating an individual's ability to mount an immune response against a tissue transplant.

Where the screening assay is a binding assay, one or more of the molecules may have a reporter molecule associated therewith, where the reporter molecule can directly or indirectly provide a detectable signal. Various reporter molecules include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labelled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

Antigen-binding molecules that are specifically immuno-interactive with DCAL polypeptides may be used in screening immunoassays, particularly to detect the interaction of agents with DCAL, or to confirm the absence or presence of a DCAL polypeptide in a cell or sample. Samples, as used herein, include biological fluids such as tracheal lavage, blood, cerebrospinal fluid, tears, saliva, lymph, dialysis fluid and the like; organ or tissue culture derived fluids; and fluids extracted from physiological tissues. Also included in the term are derivatives and fractions of such fluids. The number of cells in a sample will generally be at least about 10³, usually at least 10⁴ more usually at least about 10⁵. The cells may be dissociated, in the case of solid tissues, or tissue sections may be analyzed. Alternatively a lysate of the cells may be prepared.

For example, detection may utilise staining of cells or histological sections, performed in accordance with conventional methods. The antigen-binding molecule of interest is added to the cell sample, and incubated for a period of time sufficient to allow binding to the epitope, usually at least about 10 minutes. The antigen-binding molecule may have a reporter molecule associated therewith (e.g., radioisotopes, enzymes, fluorescers, chemiluminescers, or other labels) for direct detection. Alternatively, a second stage antigen-binding molecule or reagent is used to amplify the signal. Such reagents are well known in the art. For example, the primary antigen-binding molecule may be conjugated to biotin, with horseradish peroxidase-conjugated avidin added as a second stage reagent. Final detection uses a substrate that undergoes a color change in the presence of the peroxidase. The absence or presence of antigen-binding molecule binding may be determined by various methods, including flow cytometry of dissociated cells, microscopy, radiography, scintillation counting, etc.

An alternative assay depends on the in vitro detection of binding between antigen-binding molecules and DCAL in a lysate. Measuring the concentration of binding in a sample or fraction thereof may be accomplished by a variety of specific assays. A conventional sandwich type assay may be used. For example, a sandwich assay may first attach specific antigen-binding molecules to an insoluble surface or support. The particular manner of binding is not crucial so long as it is compatible with the reagents and overall methods of the invention. They may be bound to the plates covalently or non-covalently, preferably non-covalently. Procedures for binding of antigen-binding molecules to insoluble supports are described, for example, in U.S. Pat. Nos. 3,551,555, 3,553,310, 4,048,298 and RE-29,474. Binding of antibodies to polystyrene by adsorption has been described, for example, in U.S. Pat. Nos. 3,646,346 and 4,092,408. Binding of protein-containing antigens to a variety of insoluble supports has been described, for example, in U.S. Pat. No. 3,720,760.

The insoluble supports may be any compositions to which polypeptides can be bound, which is readily separated from soluble material, and which is otherwise compatible with the overall method. The surface of such supports may be solid or porous and of any convenient shape. Examples of suitable insoluble supports to which the receptor is bound include beads, e.g. magnetic beads, membranes and microtiter plates. These are typically made of glass, plastic (e.g. polystyrene), polysaccharides, nylon or nitrocellulose. Microtiter plates are especially convenient because a large number of assays can be carried out simultaneously, using small amounts of reagents and samples.

Cells derived from a genetically modified animal of the present invention can also be used to identify ligands or substrates that bind to, modulate, antagonize or agonize DCAL. Thus, in another embodiment, the invention contemplates a process for screening a candidate agent for the ability to specifically modulate DCAL function by (a) exposing first DC from a genetically modified non-human animal as broadly described above and second DC from a corresponding wild-type non-human animal to a candidate agent; and (b) comparing the individual immune responses of the first DC and of the second DC, wherein a candidate agent tests positive as a specific modulator of DCAL function if there is substantial modulation in the immune response of the second DC but there is no substantial modulation in the immune response of the first DC.

Changes in DC immune response can be evaluated by assessing the ability of DC to elicit a cell-mediated or humoral immune response to a target antigen. The target antigen includes, but is not restricted to, a viral antigen, a bacterial antigen, a protozoan antigen, a microbial antigen, a tumour antigen or a self- or auto-antigen. Exemplary viral antigens are derived from human immunodeficiency virus (HIV), papilloma virus poliovirus, and influenza virus, Rous sarcoma virus or a virus causing encephalitis such as Japanese encephalitis virus, a herpesvirus including, but not limited to, herpes simplex virus and Epstein-Barr virus, cytomegalovirus, a parvovirus, or a hepatitis virus including, but not limited to, hepatitis strains A, B and C. Examples of bacterial antigens include, but are not limited to, those derived from Neisseria species, Meningococcal species, Haemophilus species Salmonella species, Streptococcal species, Legionella species and Mycobacterium species. Examples of protozoan antigens include, but are not restricted to, those derived from Plasmodium species, Schistosoma species, Leishmania species, Trypanosoma species, Toxoplasma species and Giardia species. Any cancer or tumour antigen is contemplated by the present invention. For example, such antigen may be derived from, melanoma, lung cancer, breast cancer, cervical cancer, prostate cancer, colon cancer, pancreatic cancer, stomach cancer, bladder cancer, kidney cancer, post transplant lymphoproliferative disease (PTLD), Hodgkin's Lymphoma and the like. Allergenic antigens such as but not limited to house-dust-mite allergenic proteins as for example described by Thomas and Smith (1998, Allergy, 53(9): 821-832) are also contemplated by the present invention.

The amount of antigen to be placed in contact with DC can be determined empirically by persons of skill in the art. For example, DC can be incubated with an antigen, which is preferably in particulate form, for 1-2 hr at 37° C. to permit internalization and processing of the antigen. For most antigens, 10 μg/mL to 1-10 million dendritic cells is suitable for priming the dendritic cells. In a preferred embodiment, immature dendritic cells are utilized for the antigen internalization.

DC can be isolated by methods known to those of skill in the art. Suitably, DC are used from an appropriate tissue source, which is suitably blood or bone marrow. For instance, DC precursors are present in blood as peripheral blood mononuclear cells (PBMCs). Although most easily obtainable from blood, the precursor cells may also be obtained from any tissue in which they reside, including bone marrow and spleen tissue. Peripheral blood precursors may be purified using monoclonal antibodies, density gradients or centrifugation or any combination of these. Circulating frequency may be increased in vivo using flt-3 ligand. When cultured in the presence of cytokines such as a combination of GM-CSF and IL-4 or IL-13 as described below, the non-proliferating precursor cells give rise to immature DC. An exemplary method for culturing pluripotential PBMCs to produce immature DC is described by Albert et al. (International Publication WO 99/42564). In this respect, cultures of immature DC, i.e., antigen-capturing phagocytic dendritic cells, may be obtained by culturing non-proliferating precursor cells (PBMCs) in the presence of cytokines which promote their differentiation. A combination of GM-CSF and IL-4 produces significant quantities of the immature dendritic cells, i.e., antigen-capturing phagocytic or internalization-competent dendritic cells. Other cytokines that promote differentiation of precursor cells into immature DC include, but are not limited to, IL-13.

Maturation of DC requires the addition to the cell environment, preferably the culture medium, of a DC one or more maturation factors which may be selected from monocyte conditioned medium and/or factors including TNF-, IL-6, IFN- and IL-1. Alternatively, a mixture of necrotic cells or necrotic cell lysate may be added to induce maturation. Maturation can be induced in vitro using plastic adherence, cytokines, LPS, bacteria, DNA containing CpG repeats, RNA or polyIC, CD40-ligation, necrotic cells. In this regard, reference may be made to Steinman et al. (International Publication WO 97/29182) who describe methods and compositions for isolation and maturation of DC. Other methods for isolation, expansion and/or maturation of DC for the purpose of the present invention are described for example by Takamizawa et al. (1997, J Immunol, 158(5): 2134-2142), Thomas and Lipsky (1994, J Immunol, 153(9): 4016-4028), O'Doherty et al. (1994, Immunology, 82(3): 487-93), Fearnley et al. (1997, Blood, 89(10): 3708-3716), Weissman et al. (1995, Proc Natl Acad Sci USA, 92(3): 826-830), Freudenthal and Steinman (1990, Proc Natl Acad Sci USA, 87(19): 7698-7702), Romani et al. (1996, J Immunol Methods, 196(2): 137-151), Reddy et al. (1997, Blood, 90(9): 3640-3646), Thurnher et al. (1997, Exp Hematol, 25(3): 232-237), Caux et al. (1996, J Exp Med, 184(2): 695-706; 1996, Blood, 87(6): 2376-85), Luft et al. (1998, Exp Hematol, 26(6): 489-500; 1998, J Immunol, 161(4): 1947-1953), Cella et al. (1999, J Exp Med, 189(5): 821-829; 1997, Nature, 388(644): 782-787; 1996, J Exp Med, 184(2): 747-572), Ahonen et al. (1999, Cell Immunol, 197(1): 62-72) and Piemonti et al. (1999, J Immunol, 162(11): 6473-6481).

DC immune response to an antigen may be assessed using any suitable technique. For example, cytotoxic T lymphocyte (CTL) lysis assays may be employed using stimulated splenocytes or peripheral blood mononuclear cells (PBMC) on peptide coated or recombinant virus infected cells using ⁵¹Cr labeled target cells. Such assays can be performed using for example primate, mouse or human cells (Allen et al., 2000, J. Immunol. 164(9): 4968-4978). Alternatively, the DC immune response may be monitored using one or more techniques including, but not limited to, HLA class I Tetramer staining—of both fresh and stimulated PBMCs (see for example Allen et al., supra), proliferation assays (Allen et al., supra), Elispot™ Assays and intracellular INF-gamma staining (Allen et al., supra), ELISA Assays—for linear B cell responses.

G. Methods of Treatment/Prevention and Compositions Relating Thereto

The invention also encompasses a method for treatment and/or prophylaxis of a condition associated with expression or activation of DCAL, the composition comprising a modulatory agent, together with a pharmaceutically acceptable carrier. The condition is preferably selected from an allergic condition, an autoimmune disease and transplant rejection. In an especially preferred embodiment of this type, the condition is graft versus host disease or host versus graft disease. In a preferred embodiment of this type, the modulatory agent reduces the expression of DCAL or the level and/or functional activity of DCAL.

The modulatory agent can be administered to a patient either by itself, or in pharmaceutical compositions where it is mixed with suitable pharmaceutically acceptable carrier.

Depending on the specific conditions being treated, modulatory agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the agents of the invention may be formulated in aqueous solutions, preferably in physiologically compatible buffers such as Hanks′ solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The agents can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to a patient should be sufficient to effect a beneficial response in the patient over time such as a reduction in the symptoms associated with the condition (e.g., graft versus host disease or host versus graft disease). The quantity of the agent(s) to be administered may depend on the subject to be treated inclusive of the age, sex, weight and general health condition thereof. In this regard, precise amounts of the agent(s) for administration will depend on the judgement of the practitioner. In determining the effective amount of the agent to be administered in the treatment or prophylaxis of the condition, the physician may evaluate the cellular level or functional activity of DCAL and/or progression of the condition. In any event, those of skill in the art may readily determine suitable dosages of the agents of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more agents as described above with the carrier which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the modulatory agents of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes and/or microspheres.

Modulating agents of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any modulatory agent, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal inhibition of DCAL activity). Such information can be used to more accurately determine useful doses in humans.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilised. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain DCAL-modulatory effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is preferably a heart muscle tissue or a liver tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

In order that the invention may be readily understood and put into practical effect, particular preferred embodiments will now be described by way of the following non-limiting examples.

### EXAMPLES

### Example 1

### Isolation of 4b5.3 cDNA by Differential Display

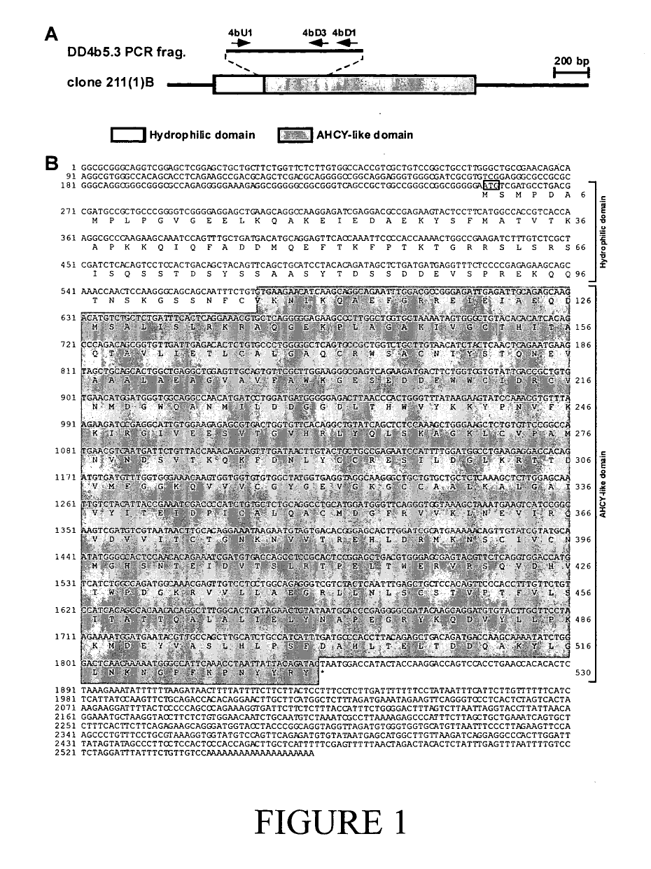

Because L428 cells have many phenotypic similarities to differentiated/activated blood DC (McKenzie et al. 1992), RAP-PCR was performed to isolate Hodgkin's disease-derived cell L428-specific transcripts. The monocytic cell line U937 activated with calcium ionophore was used as a myeloid-lineage background control. A PCR product (DD4b5.3), reproducibly identified in L428 but not in activated U937 cells, was reamplified and cloned for sequencing. DNA sequencing revealed a 416 bp insert, excluding the arbitrary primer sequences. Internal primers 4bU1 and 4bD1 (FIG. 1A) were prepared, and a preliminary RT-PCR analysis performed using RNA obtained from individual populations of normal leukocytes. This indicated that DD4b5.3 expression was restricted to DC. The 370 bp PCR fragment was then used to isolate a full length cDNA clone from an L428 cDNA library.

Methods

Cell Culture

The Hodgkin's disease-derived cell line L428 was kindly provided by Dr V. Diehl (Kinik für Innere Medizin, Cologne, Germany). The cell line U937 was obtained from the American Type Culture Collection (Rockville, Md.). Cell lines were maintained in RPMI 1640, 10% foetal calf serum (FCS), 100 U/mL penicillin and 100 μg/mL streptomycin.

RNA Preparation and Differential Display

Total RNA was extracted using Trizol (Invitorgen, Auckland, New Zealand) from L428 and U937 cells activated with A23817 calcium ionophore (500 ng/mL for 24 h; Sigma, Castle Hill, NSW, Australia), and first strand cDNA synthesised from 4 μg total RNA in a total reaction volume of 50 μL using Superscript II (Invitrogen) and 2.5 μM of an arbitrary oligonucleotide primer Zfc005 (5′-CA(C/T)TT(G/A)TA(A/G/C/T)GG(T/C)TC(A/C/G/T)CC-3′) at 42° C. for 1 h.

RAP-PCR was performed based on the methods of Welsh et al. (Welsh et al. 1992). Briefly, 4 μL aliquots of first strand cDNA from L428 and U937 cells were used as template in a PCR reaction using PCR Master (Roche Molecular Biochemicals, Auckland, New Zealand), 2 μCi [³²P]dCTP (DuPont NEN, Boston, Mass.) and 1 μM Zfc005 in a total volume of 20 μL. Samples were placed in a thermocycler (PTC-200, M J Research, Waltham, Mass.) and a two stage PCR thermocycle used: Samples were first denatured at 94° C. for 2 min, followed by five cycles of a “low stringency” PCR profile of 94° C. for 1 min, 40° C. for 1 min and 72° C. for 1 min, followed by 30 cycles of a “high stringency” PCR profile of 94° C. for 1 min, 58° C. for 1 min and 72° C. for 1 min. Lastly, samples were incubated at 72° C. for a further 5 min, and stored at 4° C. until analysis. RAP-PCR products were fractionated on 6% polyacrylamide non-denaturing gels. The gels were vacuum dried on 3MM paper without fixation and exposed to X-ray film. RAP-PCR was repeated to ensure reproducibility of bands. The autoradiograms were then used as guides to excise candidate bands specific for L428 but not for U937 from the dried sequencing gels. Excised bands were rehydrated in sterile water and used as template in PCR reaction to reamplify the products with the arbitrary primer. The resultant PCR products were gel-purified and ligated into the pGEM-T cloning vector (Promega, Madison, Wis.) for sequencing. An L428 specific clone (DD4b5.3) was selected and studied further.

### Example 2

### DD4b5.3 cDNA Encodes an S-adenosylhomocysteine Hydrolase-Like Molecule

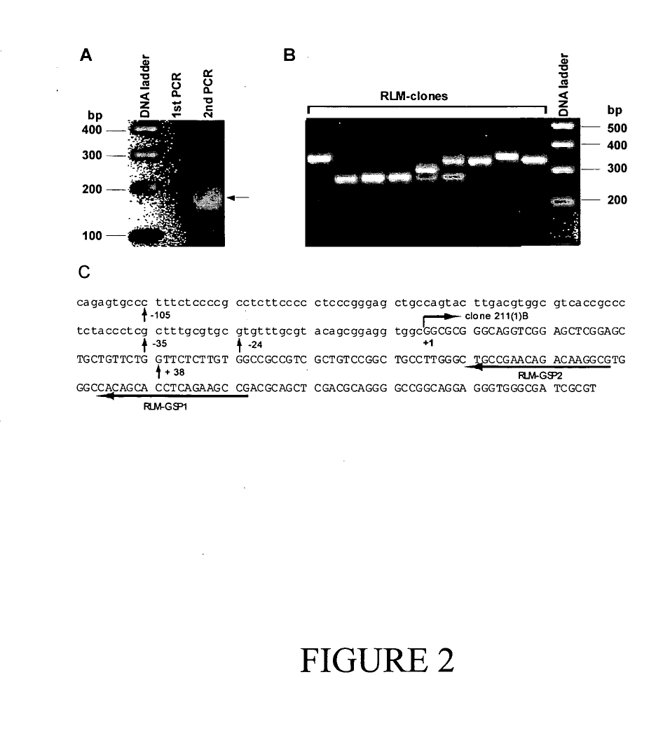

Probing the L428 cDNA library with the DD4b5.3 probe isolated a number of overlapping clones. The longest DD4b5.3 clone (clone 211(1)B) contained a 2563 bp insert (FIG. 1), and the longest open reading frame obtained by assigning a stop codon (TAA) at nucleotides 1845-1847. 5′-Rapid Amplification of cDNA end (RACE) was performed to characterize the 5′-untranslated region of the DD4b5.3 cDNA. Two consecutive PCR amplifications resulted in an approximately 160 bp band (FIG. 2A). Subsequent cloning and sequencing of the PCR product indicated multiple transcription start sites (+38, −24, −35 and −105, relative to the 5′ end of clone 211(1)B) (FIGS. 2B and C). As no in-frame stop codons were found at the 5′-end, it appeared that one of a cluster of three in-frame ATGs (at nucleotides 255-275 in FIG. 1B) encoded the initiation codon (FIG. 2A). Strikingly, the putative mouse DCAL protein was 100% identical to the human homologue (data not shown). The putative amino acid sequence of DD4b5.3 contained 530 amino acids (aa) with two distinct domains, a hydrophilic domain (1-106 aa) and adenosine-homocysteine hydrolase (AHCY) (Coulter-Karis, Hershfield 1989)-like domain (107-530 aa). As DD4b5.3 was predominantly expressed in DC (described below), we named DD4b5.3 as DC-expressed AHCY-like molecule (DCAL). Northern blot analysis detected a single ˜4.4 kb band for DCAL in L428 (data not shown). The DCAL gene was localised to chromosome 1 by PCR screening genomic DNA from a panel of monochromosomal somatic cell hybrid cell lines (data not shown) and further to chromosome band 1p21.1 using the Human Genome database.

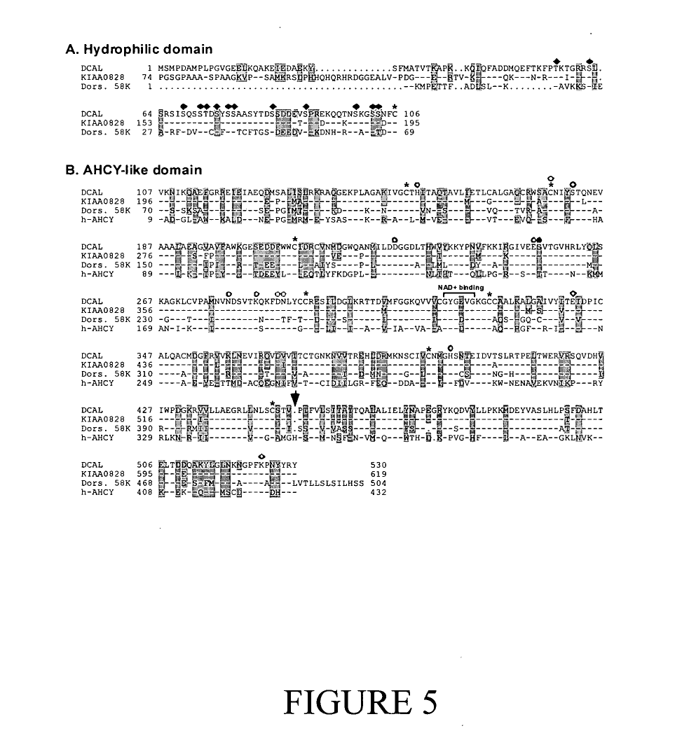

NCBI BLAST searching of the non-redundant GenBank database revealed the significant similarity of DCAL to two AHCY-like sequences with unknown function, human KIAA0828 (GenBank accession number AB020635) isolated from a brain cDNA library (Nagase et al. 1998) and Drosophila bithorax complex derived 58K hypothetical protein (SwissProt accession number S01302) (DeLorenzi et al. 1988) (FIG. 5). The two AHCY-like molecules also contained a hydrophilic domain attached to the N-terminus of an AHCY-like domain. The hydrophilic domain of DCAL contained 76 polar or charged residues, including the region from amino acid position 62 to 106 (45 aa) that contained 16 serine residues and only six non-polar residues. The DCAL hydrophilic domain shared 66.7% and 40.3% amino acid identity to KIAA0828 and Drosophila 58K protein, respectively. Ten serines in the DCAL hydrophilic domain were conserved in both KIAA0828 and Drosophila 58K protein, suggesting that this domain may mediate an important regulatory function such as serine phosphorylation by serine kinases (FIG. 5A).

The amino acid identity of the AHCY-like domain of the putative DCAL, KIAA0828 and Drosophila 58K proteins to human AHCY was 51.5%, 51.1% and 51.5%, respectively, whereas the amino acid identity of the AHCY-like domain of DCAL to that of KIAA0828 and Drosophila 58K protein is 93.6% and 73.9%, respectively, suggesting that DCAL is more closely related to KIAA0828 and Drosophila 58K protein than AHCY (FIG. 5B). Importantly, amino acids believed to be important for AHCY function, including the seven of eight cysteines and residues involved in NAD⁺ binding and ribose moiety binding, are conserved between human AHCY and DCAL, and are also conserved in the other AHCY-like molecules (FIG. 5B).

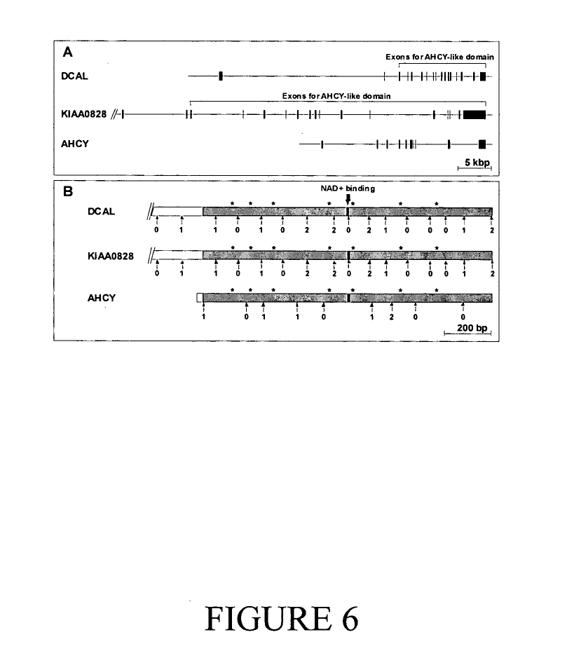

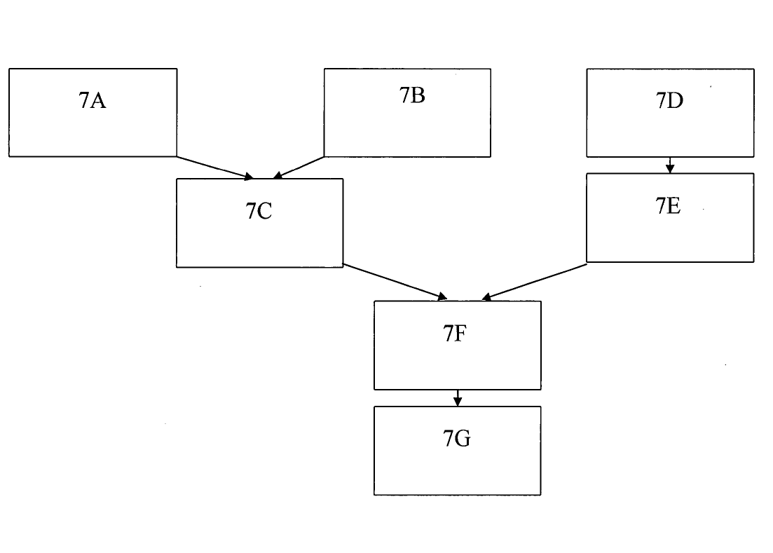

The inventors identified bacterial artificial chromosome (BAC) clones containing genes for DCAL (accession number AL355817, chromosome 1 clone), KIAA0828 (accession number AC009244, chromosome 7 clone) and AHCY (accession number AL137253, chromosome 20 clone) by NCBI BLAST searching of the HTGS database (FIG. 6). Whilst the AHCY gene (˜23 kb) was encoded by 10 exons, both DCAL (>37 kb) and KIAA0828 genes (>51 kb) were encoded by more than 17 exons in which the downstream 15 exons contained the AHCY-like domains. This indicates that the exon-intron junction organization of the DCAL and KIAA0828 AHCY-like domains differ from that of AHCY (FIG. 5A). AHCY gene structure was similar to that of the rat homologue in both number and size of exons and exon organization (Merta et al. 1995). The difference between AHCY and the AHCY-like genes became more apparent when the positions and phases of exon-intron junctions were assessed by cDNA analysis (FIG. 6B). Strikingly, both DCAL and KIAA0828 shared identical positions of exon-intron junctions and phases throughout the molecules, including that the most 3′ exon contained only the last amino acid (tyrosine) and that the NAD⁺ binding site was encoded by two exons. In contrast, the AHCY NAD⁺ binding site was encoded by a single exon (FIG. 6B). The gene structure of mouse DCAL is identical to that of human homologue. These data suggest that both DCAL and KIAA0828 are evolutionally distinct from AHCY, and belong to a family of AHCY-like molecules.

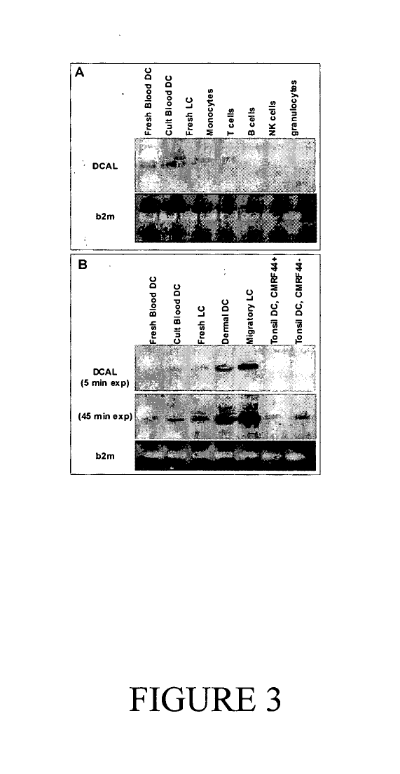

AHCY is involved in methylation, an important cellular regulatory mechanism. Protein methylation can lead to the activation or deactivation of target proteins, DNA/RNA methylation affects transcription and translation, while lipid methylation may modulate lipid membrane function. The cellular methylation pathway exclusively uses S-adenosylmethionine (SAM) as a methyl donor, resulting in adenosine-homocysteine (SAH), which is inhibitory to the trans-methylation reaction (Chiang et al. 1996). AHCY (EC 3.3.1.1) catalyses the reversible hydrolysis of SAH to adenosine and homocysteine. The homozygous AHCY-deficient mouse embryo dies around time of implantation, indicating the importance of this enzyme during embryonic development (Miller et al. 1994). The AHCY molecule is described as a homotetramer having a tight globular structure maintained by intramolecular bonds. Seven of eight Cys residues, which mediate disulfide bonds, were conserved between human AHCY and DCAL (FIG. 3). This included Cys¹⁹⁵ (Cys²⁹³ in DCAL) shown to be vital for enzymatic function (Yuan et al. 1996).

Crystallographic studies have shown that the human AHCY appears to be an asymmetric dimer, with the monomer structure reminiscent of other NAD-dependant dehydrogenases such that the NAD⁺ binding and catalytic sites are located in the cleft between two globular domains (Turner et al. 1998). However, in contrast to the NAD⁺-dependent dehydrogenases, both subunits are involved in a single NAD⁺ binding site, with the Lys⁴²⁶ and Tyr⁴³⁰ residues of one subunit contributing to the NAD⁺ binding site of the other, a configuration thought to be unique to AHCY. These amino acids are conserved in DCAL (Lys⁵²⁴ and Tyr⁵²⁸), as is the NAD⁺ binding motif (GlyXGlyXXGly) at amino acid positions 318 to 323 and the acidic amino acid 18 position C-terminal to the motif (Glu³⁴¹).

Adenosine binding to AHCY is mediated by 15 amino acids (Turner et al. 1998). Ten residues that interact primarily with the ribose moiety of adenosine (His⁵⁵, Cys⁷⁹, Ser⁸³, Asp¹³¹, Glu¹⁵⁶, Thr¹⁵⁷, Asn¹⁸¹, Lys¹⁸⁶, Asp¹⁹⁰, Asn¹⁹¹, His³⁰¹ in AHCY) were all conserved in DCAL except for AHCY Thr¹⁵⁷, which is conservatively replaced with Ser in DCAL. Interestingly, the four amino acids in ACHY (Thr⁵⁷, Leu⁵⁴, Glu⁵⁹ and His³⁵³) involved in the binding of the adenine ring were not well conserved in DCAL. Likewise, amino acids in AHCY (Phe³⁰² and Asp³⁰³) predicted to interact with the homocysteine portion of SAH were also poorly conserved. Site directed mutagenesis studies have shown that Lys⁴²⁶ in AHCY is critical for enzymatic function (Ault-Riche et al. 1994) and was conserved in DCAL (Lys⁵¹⁸).

Analysis of the predicted functional domains in AHCY suggested that DCAL, as well as KIAA0828 and Drosophila 58K protein, may retain some AHCY functions such as NAD⁺ binding. Whilst the residues involved in the ribose moiety binding appeared intact, those binding to the adenine and homocysteine portions of SAH did not, suggesting these AHCY-like molecules may bind other nucleotide or nucleotide-like molecules.

Methods

cDNA Library Screening

An oligo dT-primed L428 cDNA library was prepared using the ZAP Express cDNA Gigapack Cloning kit (Stratagene, La Jolla, Calif.) according to the manufacturer's instructions. The library was screened by plaque hybridization with DD4b5.3-specific 370 bp PCR fragment amplified with primers 4bU1 (5′-CAACTCCAAGGGCAGCAGCAA-3′ at nucleotide 545-565) and 4bD1 (5′-CCCATCCATGTTCACACAGC-3′ at nucleotide 895-914) (see FIG. 1) labeled with [α-³²P]dCTP using the Multiprime™ DNA labeling system (Amersham Pharmacia, Auckland, New Zealand). The final washes were performed in 0.5×SSC, 0.1% SDS at 65° C. Positive plaques were plaque purified twice and converted to the pBK-CMV phagemid (Stratagene) according to the manufacturer's instructions.

DNA Sequencing and Computer Analysis

DNA sequencing was performed by the dideoxy chain termination method using either the Thermo Sequenase™ Primer Cycle Sequencing kit (Amersham Pharmacia) with IRD₄₀-labelled primers (MWG,-Biotech, Ebersberg, Germany) separated on a Li-COR automated sequencer (Lincon, Nebr.) in house or a BigDye Terminator kit on a ABI Prism 377 automated sequencer (PE Applied Biosystems, Scoresby, VIC, Australia) by Australian Genome Research Facility (University of Queensland, St. Lucia, QLD, Australia). The National Center of Biotechnology Information (NCBI) Center BLAST was used to search either for homologous sequences (non-redundant data base) or for genes (HTGS database). Sequence alignments and motif searches were performed using the Bestfit and Motifs programs, respectively, from the GCG computer package (Madison, Wis.) or from Australian National Genomic Information Service (Sydney, NSW, Australia). To clone mouse DCAL cDNA, GenBank mouse non-redundant and expressed sequence tagged database were searched with the putative coding sequence of human DCAL using BLASTN algorithm. A contig containing an open reading frame coding for mouse DCAL was assembled from 6 distinct ESTs (accession numbers AW229870, AW318709, BF133923, BF167887, BF783516, and BF789392) and the sequence further confirmed using additional 19 ESTs. Potential phosphorylation sites were predicted using NetPhos 2.0 program (www.cbs.dtu.dk/services/NetPhos/) (Blom et al. 1999).

5′-Rapid Amplification of cDNA End (RACE)

5′-RACE was performed using a FirstChoice™ RLM-RACE kit (Ambion, Austin, Tex.). Briefly, total RNA from L428 was treated sequentially with calf intestinal alkaline phosphatase and tobacco acid pyrophosphatase to select and to remove cap structure of full length mRNA. The RNA adaptor was ligated to the RNA using T4 RNA ligase, and the RNA was subjected to cDNA synthesis with random decamer and Thermoscript reverse transcriptase (Invitrogen). Resultant cDNA was subjected to two rounds of PCR using DCAL specific primer RLM-GSP1 (5′-CGGCTTCTGAGGTGCTGT-3′ at the nucleotide 92-119) and RLM-GSP2 (5′-CGCCTTGTCTGTTCGGCA-3′ at the nucleotide 77-94) in combination with the 5′RACE outer primer and inner primer, respectively. The PCR product was cloned into pGEM-T easy vector and sequenced.

### Example 3

### RT-PCR Analysis

DCAL expression in leukocytes was assessed by semi-quantitative RT-PCR on FACS-sorted leukocyte populations (purity >95%), followed by Southern blot analysis using a DCAL-specific internal oligonucleotide probe (FIG. 3). Peripheral blood DC, cultured overnight to give rise to the differentiated/activated CMRF-44⁺ phenotype, expressed abundant DCAL mRNA, whereas fresh blood DC (lin⁻ HLA-DR⁺) and freshly isolated LC expressed considerably less DCAL mRNA (FIG. 3A). Expression of DCAL mRNA was weak or undetectable in the other normal leukocyte populations tested; CD14⁺ monocytes, CD3⁺ T lymphocytes, CD19⁺ B lymphocytes, CD16⁺ NK cells and CD57⁺ granulocytes.

Given the apparent differences in DCAL mRNA levels according to blood DC differentiation/activation state, the inventors undertook a more extensive RT-PCR analysis of different DC populations (FIG. 3B). Again, comparatively low levels of DCAL mRNA were detected in fresh peripheral blood DC, whereas moderate to high levels of DCAL mRNA were detected in activated blood DC and dermal DC. Migrating LC appeared to express higher levels of DCAL mRNA compared with freshly isolated LC. Interestingly, both CMRF-44⁺ and CMRF-44⁻ tonsil DC populations, reflecting later stages of DC differentiation/activation, expressed only low levels of DCAL mRNA.

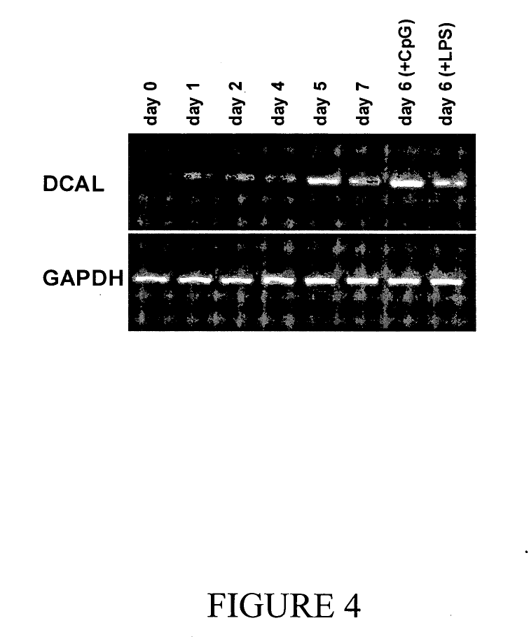

DCAL expression was further investigated in CD14⁺ monocytes differentiated into monocyte-derived DC (Mo-DC) by culture in cytokines GM-CSF, IL-4 and TNF-α (FIG. 4). DCAL mRNA expression was undetectable in freshly isolated monocytes, and increased only slightly after 24 hour culture. Low levels of DCAL expression were maintained up to day 4. DCAL mRNA increased markedly when monocyte became differentiated to Mo-DC on day 5-7. Further activation of immature MoDC using CpG-containing oligonucleotides or LPS did not appreciably change the DCAL mRNA levels.

In summary, DCAL was expressed by fresh and cultured peripheral blood DC and freshly isolated skin LC, but showed little or no expression in other PBMC, including monocytes, T and B lymphocytes, NK cells and granulocytes. Examination of a more extensive DC lineage panel revealed that DCAL expression was regulated during DC differentiation/activation. DCAL expression in dermal DC was much stronger than in activated peripheral blood DC, while migratory LC (McLellan et al. 1998) showed the strongest DCAL expression. Interestingly, DCAL mRNA expression was diminished in terminally differentiated tonsil DC (lin⁻, DR⁺). Increased DCAL mRNA expression during DC differentiation/activation could be demonstrated during monocyte differentiation to Mo-DC using cytokines. While no consistent decrease in DCAL mRNA was observed in Mo-DC matured in the presence of TNF-α, this may possibly reflect the fact that the in vitro-derived cells did not receive negative regulation.

Methods

Purification of Normal Leucocyte Cell Populations

Freshly isolated “resting” blood DC and differentiated/activated blood DC were obtained as previously described (Fearnley et al. 1997). Briefly, fresh blood DC were purified from T lymphocyte-depleted peripheral blood mononuclear cell (PBMC) preparations by negatively selecting against CD3, CD11b, CD14, CD15, CD16 and CD19 and positively selecting for HLA-DR by fluorescence activated cell sorter (FACS). Differentiated/activated blood DC, represented by the CMRF-44⁺, CD14⁻, CD19⁻ population, were obtained after FACS purification from the Nycodenz™ low density fraction of T lymphocyte-depleted PBMC cultured overnight in RPMI 1640 and 10% FCS.

Monocyte, B lymphocyte and NK cell populations were FACS sorted from T lymphocyte depleted PBMC preparations on the basis of positivity for CD14, CD19 and CD16, respectively. T lymphocytes were purified by sheep red blood cell rosetting, the red blood cells lysed in H₂O and the remaining cells sorted for CD3⁺ by FACS. Granulocytes were purified from PBMC depleted red blood cell fraction by centrifugation in 5% dextran in PBS. After the dextran centrifugation, the cell pellets were collected and granulocytes obtained by FACS on the basis of forward and side scatter characteristics and CD57 positivity. Granulocyte preparations were processed for RNA as quickly as possible to avoid autocatalysis of the cell contents.

LC were isolated from human breast skin as described previously (McLellan et al. 1998). Briefly, epidermal sheets were prepared by digesting split thickness breast skin with dispase II (Roche Molecular Biochemicals, Auckland, New Zealand). Epidermal cell suspension was obtained by mechanical and enzymatic (trypsin and DNaseI for 20 min at 37° C.) dissociation of the epithelial sheets, and LC were enriched over Lymphoprep gradient (Amersham Pharmacia Biotech). LC were sorted on the basis of positivity for HLA-DR.

Migratory LC were obtained by culturing small pieces of epidermal sheets (1-2 mm³) in petri dishes containing RPMI 1640 with 10% FCS and 25 mM HEPES (pH 7.4) as described previously (McLellan et al. 1998). After 2 days culture, the cells in media were collected by filtration, enriched by Nycodenz™ gradient and HLA-DR bright cells sorted by FACS. Dermal DC were obtained from human dermal sheets, minced by scalpel, treated with 1.0 mg/mL collagenase (Type D, Roche Molecular Biochemicals) and DNase I (Sigma, Castle Hill, NSW, Australia) in media as described previously. Cells were collected by filtration, washed and dermal DC purified by Lymphoprep™ gradient and HLA-DR strong positive cells sorted by FACS.

Tonsil DC were obtained from human tonsil tissue minced using scissors and dissociated by repeated passes through a syringe (Hart and McKenzie 1988). Cell debris was settled out and discarded and the remaining cells passed over a Ficoll Hypaque gradient, T lymphocytes removed by sheep erythrocyte rosetting and DC isolated by FACS sorting for lin⁻, DR⁺ cells as described for peripheral blood DC isolation. Tonsil DC were further sorted into CMRF-44⁺ and CMRF-44⁻ populations (Summers et al. 2001).

Monocyte derived DC (Mo-DC) were prepared according to previously published methods (Vuckovic et al. 1998). Briefly, CD14⁺ monocytes were cultured for 6-7 days in media containing 800 U/mL GM-CSF (Sandoz-Pharma, Auckland, New Zealand) and 500 U/mL IL-4 (Sigma Chemicals, St Louis, Mo.). Further functional maturation was achieved by adding 20 ng/mL TNF-α (Hoffman LaRoche, Basel, Switzerland) or 1 μg/mL lipopolysaccharide (LPS, Sigma) in the last two days of culture. Alternatively, 5 μM CpG-containing oligonucleotide (5′-GGAATGTCGATGCCTGACGCGATGCCGCTG-3′, CpG underlined) was added on day 0, 3 and 4, and harvested on day 6. The cells sorted for CMRF-44 or unsorted were subjected to RT-PCR analysis.

The purity of the FACS sorted cell populations were greater than 95%.

RT-PCR Analysis

Total RNA was prepared from FACS-purified leukocytes (5×10³−2×10⁵ cells) using the RNeasy RNA extraction kit (Qiagen, Clifton Hill, VIC, Australia). RNA was collected in DEPC water, volumes adjusted to a final concentration of 100 cell equivalents/μL, and stored at −80° C. until use. First strand cDNA was synthesized using SuperScript II in a reaction volume of 40 μL containing an oligo d(T)₁₈ primer and 20 μL RNA at 42° C. for 1 hr.

DCAL specific RT-PCR was performed using primers 4bU1 and 4bD1 with 2.5 μL cDNA template to give 125 cell-equivalents. Amplification was performed on a thermocycler using a profile of 94° C. for 2 min, then 30 cycles of 94° C. for 1 min, 59° C. for 1 min and 72° C. for 1 min, followed by a final step at 72° C. for 5 min. In some experiments, the agarose gel fractionated DNA was transferred onto Hybond N+ membranes (Amersham Pharmacia Biotech) and probed with an digoxigenin (DIG)-labeled DCAL-specific primer 4bD3 (5′-TCAGCCAGTGCTGCAGCTAC-3′ at nucleotide 810-829) and the signal detected with CDP-Star (Roche Molecular Biochemicals) on X-ray film. To normalize the cDNA input per PCR, control PCRs were performed to amplify either a 196 bp β₂-microglobulin PCR fragment using primers β₂mU1 (5′-TTAGCTGTGCTCGCGCTACTCT-3′) and β₂mD1 (5′-TCGGATTGATGAAACCCAGACA-3′) or 574 bp glyceraldehyde-3-phosphate dehydrogenase (GAPDH) fragment as described previously (Kato et al. 2000).

### Example 4

### Vector Construction for a Mouse DCAL Knockout: Construct A

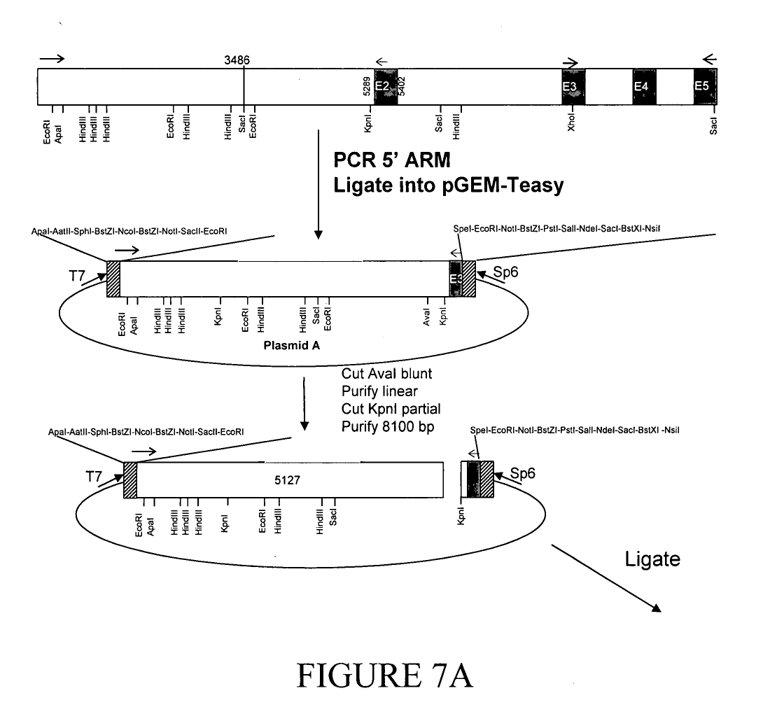

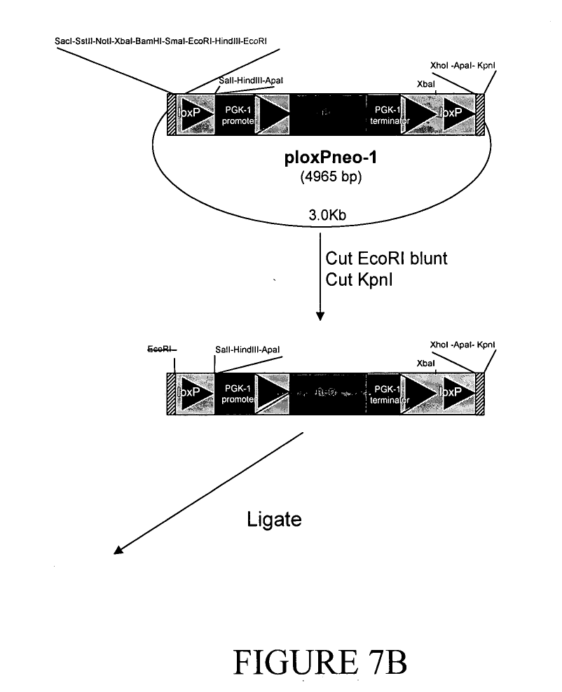

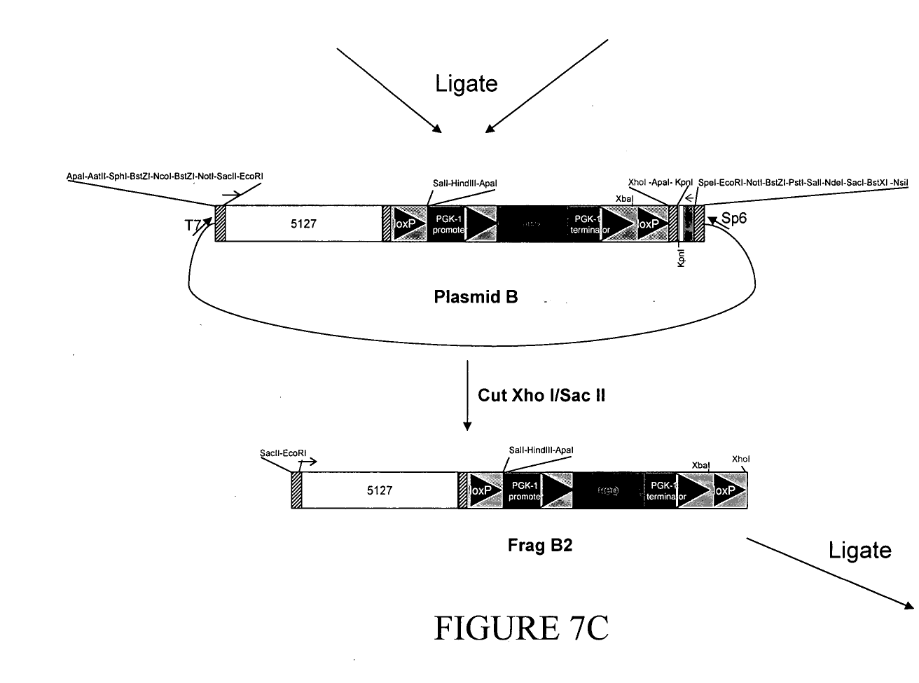

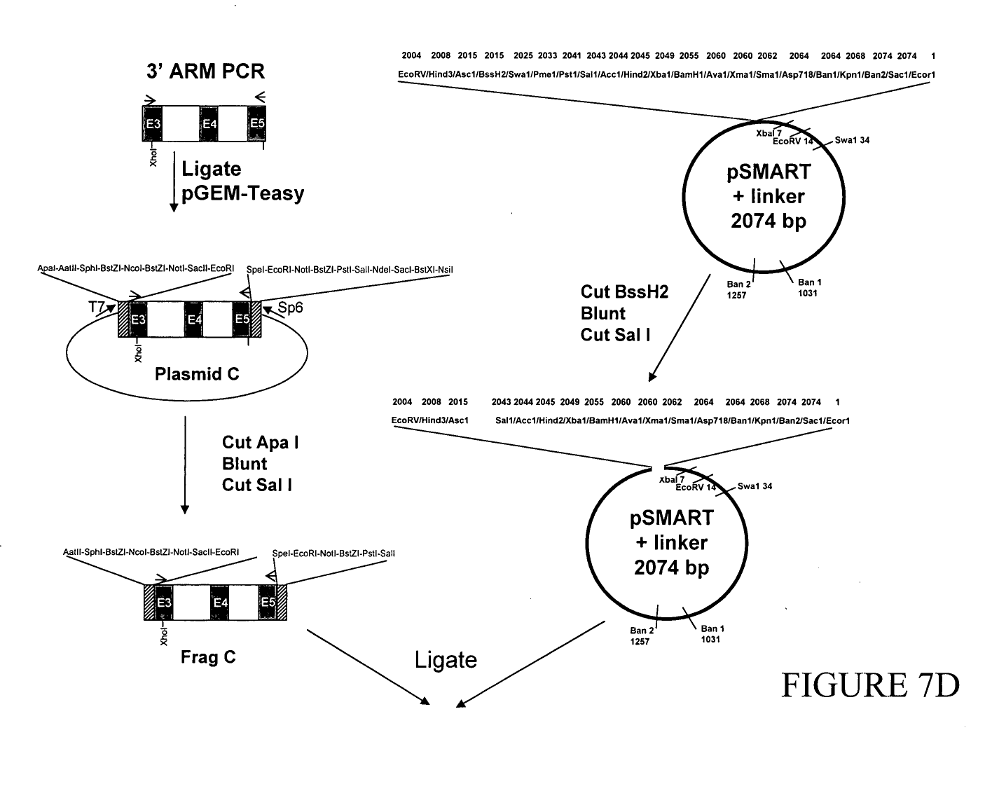

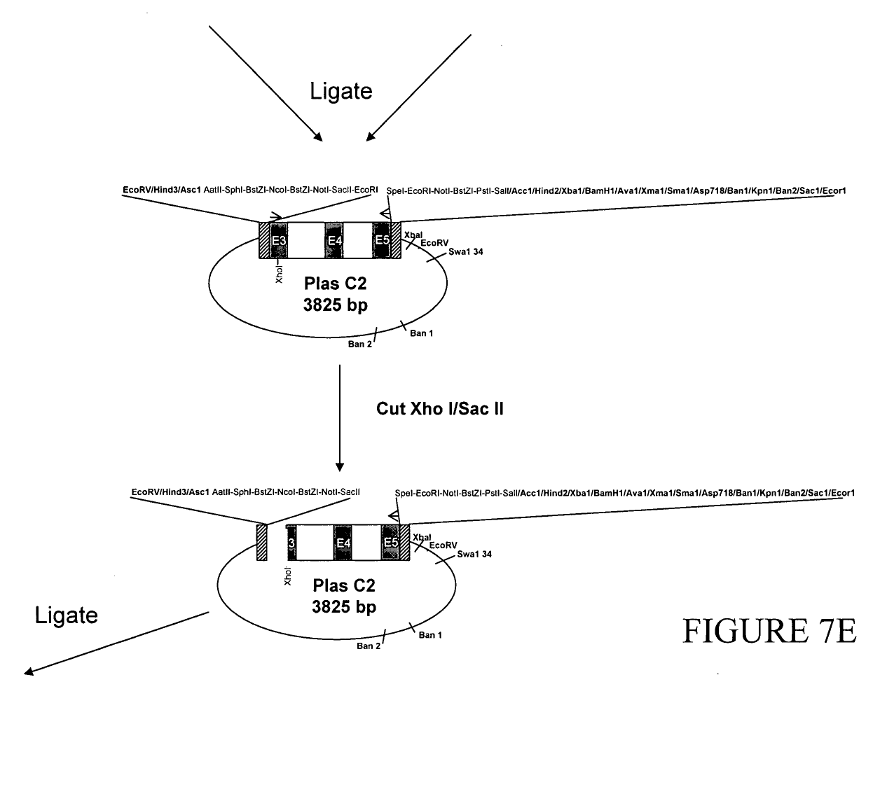

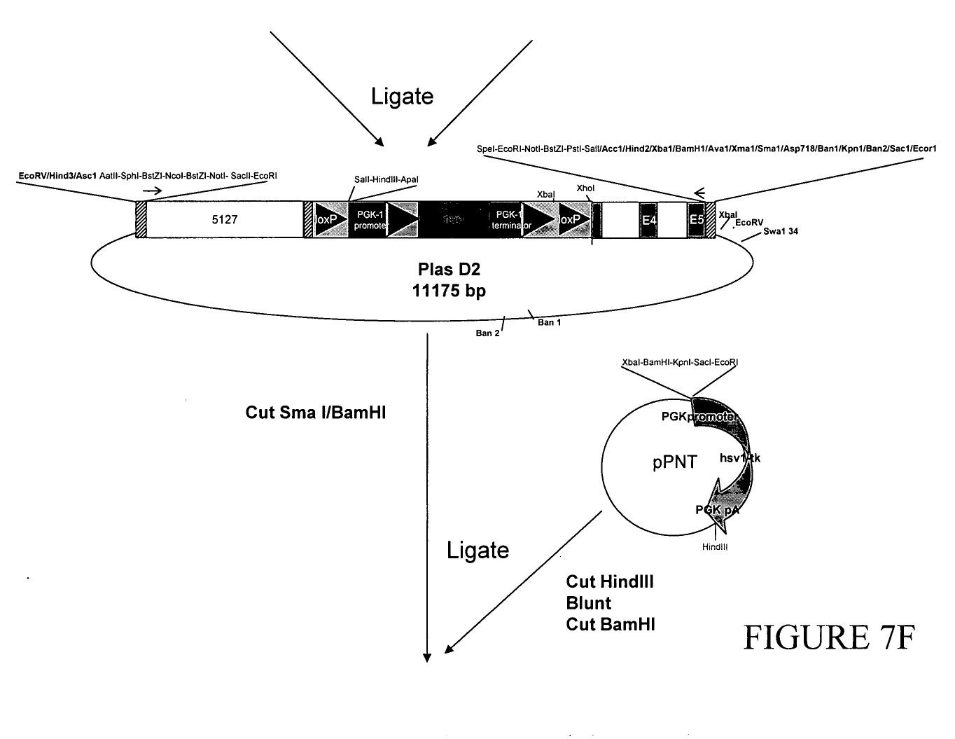

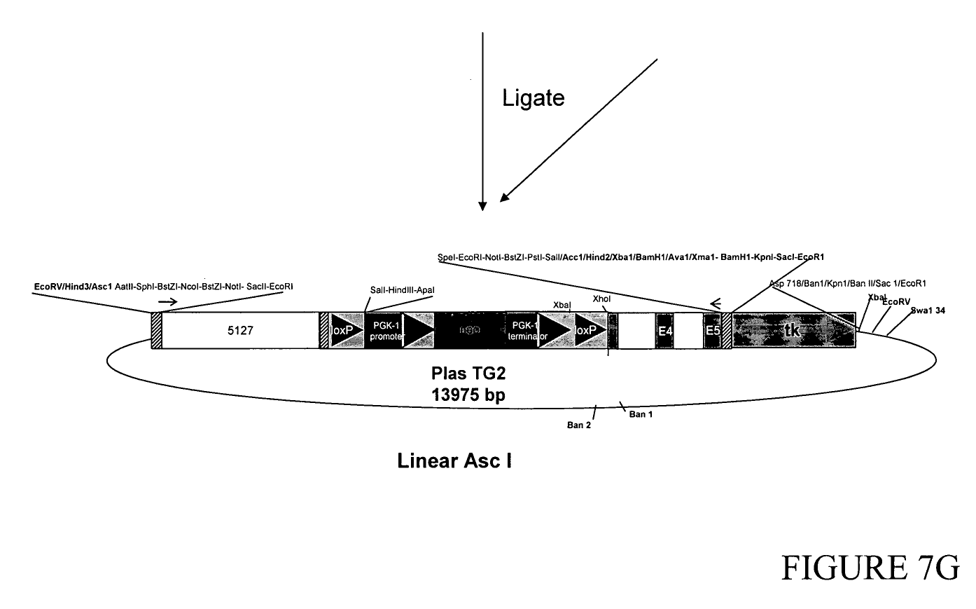

With reference to FIGS. 7A-7G, a 5.3 kb PCR fragment spanning Intron 1 and Exon 2 of the mouse DCAL (mDCAL) gene is generated using long range PCR to create a 5′ transgene (TG) arm. A 1.7 kb PCR fragment spanning Exon 3 to Exon 5 is also generated to create a 3′ TG arm. To ensure no alternate splice events reconstitute a partial protein of the transgene Exon 5 primers are designed with an engineered stop site. The 5′ TG arm is then ligated into the pGEM-Teasy vector, resulting in Plasmid A. Plasmid A is restricted with AvaI, blunted with T4 DNA polymerase I and partially restricted with Kpn I. The ploxPneo-1 plasmid is restricted with EcoI, blunted and restricted with KpnI, and the insert containing a Neo gene cassette from this plasmid is ligated into the blunted AvaI and KpnI site of Plasmid A to form plasmid B. Plasmid B is then cleaved with XhoI and SacII restriction enzymes and the plasmid insert purified. The PCR amplified 3′ TG arm is ligated into pGEM-Teasy vector. The 3′TG arm plasmid is restricted with ApaI, blunted and restricted with SalI and the insert purified from the plasmid backbone. The PSMART™ vector is restricted with BssH2, blunted and restricted with SalI. This restricted vector is ligated with the blunted ApaI-SalI fragment of the 3′TG arm to prepare Plasmid C2, which is restricted with XhoI and SacII, and ligated with the XhoI and SacII restricted insert of Plasmid B to generate Plasmid D2. The pPNT vector is restricted with HindIII, blunted and restricted with BamHI, and a fragment containing the TK gene cassette is purified. This fragment is then ligated to SmaI and BamHI arms of Plasmid D2 to generate Plasmid TG2 (pTG2-DCAL). This plasmid is a null construct, which causes deletion of exon 2 and a frame shift in the resulting coding region.

### Example 5

### Vector Construction for a Conditional Mouse DCAL Knockout: Construct B

The presence of mDCAL expression in vital organs, including the brain and kidney, may require the ability to knock out mDCAL in a temporal or tissue specific or inducible manner using Cre-lox sites flanking mDCAL sequence. To allow this, the construct may also be produced as outlined above with the neo section replaced with the gene sequence from Intron2-Exon 3, which is the section excised from Construct A outlined above.

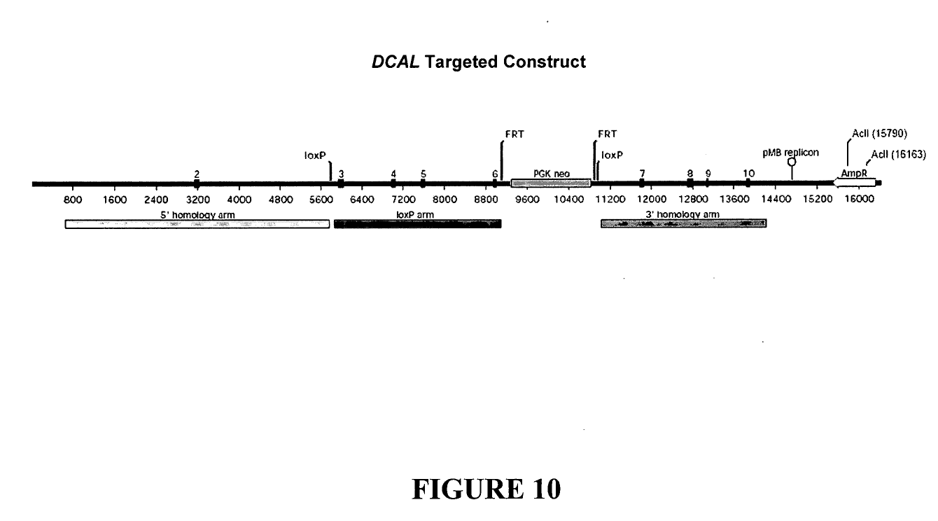

Alternatively, the construct may be designed with exons 3 to 6 of the mDCAL gene flanked with loxP sites to allow Cre-mediated deletion of these exons (see FIG. 10). The N-terminal domain (NTD) has been shown to be involved in the binding of AHCY-L1 to the phosphatidyl inositol receptor. Splicing of exon 2 to 7 should cause a frame-shift mutation with the introduction of an early stop codon. In this illustrative targeting vector, the PGK-neo selection cassette is inserted downstream of exon 6 and is flanked by FRT sites, through which the cassette can be deleted using FLPe recombinase. Optionally, the PGK-neo cassette and exons 3 to 6 could be deleted together in a single step using Cre recombinase. The targeting vector is constructed from three fragments, the 5′ homology arm, the 3′ homology arm and the loxP arm. The 5′ homology is approximately 4 kb to 5 kb in length and the 3′ homology arm is approximately 3 kb in length.

### Example 6

### Introduction of Knock-Out Construct to ES Cells

ES cells (1.5-2×10⁷ cells) from a 2-day culture are harvested, washed once in PBS and resuspended in cold PBS to a final concentration of 1.2×10⁷ cells/mL. To two Biorad™ cuvettes A and B are added 0.9 mL (10⁷ cells) of the resuspendate and 0.167 mL (2×10⁶ cells ) of the resuspendate+0.73 mL PBS, respectively. To cuvette A is added 15 μg linearized plasmid DNA, immediately prior to electroporation. To cuvette B is added no DNA. Using a Biorad Gene Pulser™ according to the method of McMahon and Bradley (1990, Cell 62: 1073-1085), each cuvette is twice subjected to an electric pulse i.e. 500 μF 230V then 500 μF 240V. Using a pasteur pipette, the cells are transferred as soon as possible: from cuvette A to a 10 mL tube containing 4 mL medium, then plated at a rate of 5×1 mL cells onto five 10 cm diameter petri dishes (labeled plates 4-8) containing NeoPMEFs and 10 mL medium; and from cuvette B to a 10 mL tube containing 1 mL medium, then plated all onto a 10 cm diameter petri dish (labeled plate 2) containing NeoPMEFs and 10 mL medium.

The following control plates are also set up: a) plate 0.167 mL (2×10⁶ cells) original cell suspension onto a 10 cm diameter petri dish (labeled plate 3) containing NeoPMEFs and 10 mL medium.; and b) dilute 0.167 mL (2×10⁶ cells) in 10 mL medium (˜2×10⁵ cells/mL) and plate 0.1 mL (2×10⁴ cells) onto Neo PMEFs in 10 cm petri dish (labeled plate 1) with 10 mL medium. All plates are incubated in a CO₂ incubator overnight.

Next day approx. 24 hrs after electroporation plates receive appropriate selection medium as specified in TABLE 2.

TABLE 2

| | No of | | Media | |
|---|---|---|---|---|
| Plates | Cells | EP Treatment | Treatment | |
| 1 | 2 × 104 | No EP | Normal | Counting & plating |
| | | | | efficiency |
| 2 | 2 × 106 | EP No DNA | Normal | % survival after EP |
| 3 | 2 × 106 | No EP | G418 | % G418 resistant clones in |
| | | | | stock |
| 4 | 2 × 106 | EP + DNA | Normal | % survival after EP + DNA |
| 5 | 2 × 106 | EP + DNA | G418 | % total integrants i.e., |
| | | | | random + H/Rs |
| 6 | 2 × 106 | EP + DNA | G418* | Same as Plate 5 |
| 7 | 2 × 106 | EP + DNA | G418* | Same as Plate 5 |
| 8 | 2 × 106 | EP + DNA | G418* | Same as Plate 5 |

The medium is changed on all plates every 48 hrs. By 3 days non G418 resistant clones are non viable, after one week there should be no clones on Plates 2 & 3. If there are clones present the experiment is invalid. After 6-7 days the colonies on plates 5-8 (plates 6-8 if PNS is used) are ready for harvesting. For efficient picking of single clones there should be no more than approx. 100 colonies/plate.

Single colonies are selected using the following procedure. Plates are treated one at a time and as quickly as possible. Medium is removed and cells washed with PBS. A small volume of PBS is then added to the plate, and the plate is held over a piece of black cardboard. Using a P-200 Gilson™ pipette single colonies are picked whilst minimizing the volume of PBS taken with each colony. Six colonies are picked in succession and placed into a 96 well plate containing 20 μL trypsin/EDTA. A microscope is used to check that there are single colonies in each well. Using a multichannel pipette colonies are dispersed by pipetting up and down 40× in a circular motion, being careful not to cause bubble formation as this causes cell death, and then checking that the colonies have dispersed. Cells are then transferred to a 96 well tray, containing Neo PMEFs. This process is repeated until all colonies are picked. After 3-4 days, clones are be ready for splitting and freezing down.

The following procedure is employed for splitting and crypreserving clones. Medium is removed one row at a time using an aspirator and a sterile pasteur pipette. Each well is washed with 0.1 mL PBS, and subsequently with 0.1 mL PBS/EGTA. Forty microlitres of trypsin/EDTA is then added to each well and cells dispersed using a multichannel pipette disperse cells as before. Twenty-five microlitres of the resuspended cells are then transferred into a 96 well plate containing 10% DMSO in medium. A Gilson™ pipette is used with a sterile tip to transfer the remaining cells into a 24-well plate with 1 mL medium+LIF (no feeders) per well, wherein each well has been pre-treated with 0.5 mL gelatin/PBS. These cultures are set aside for DNA isolation and Southern blot analysis.

Once entire tray is complete, the 96 well tray is sealed using tape, occupied wells are recorded on record sheet and tray is then placed into a pre-chilled foam box at −70° C. Once cultures are frozen (>2 hrs) the trays are transferred to a thermoinsulated container at −80° C. and stored until Southern analysis is complete.

### Example 7

### Isolation of DNA from 24 Well Plates

Once cultures are almost confluent, they are ready for harvesting. The medium is removed using an aspirator and a pasteur pipette and each well is washed twice with 0.5 mL PBS. Two-hundred microlitres of 6M Guanidine HCl/NaAc solution is then added to each well and cells lyse almost immediately. Using a pasteur pipette and a rubber teat, the viscous solution is slowly drawn up the pipette. The solution is then transferred into a labeled Eppendorf™ tube. The tubes are then taped into an Eppendorf rack and taped to a revolving wheel to mix overnight. The following day, in lots of 12 or 20 depending on size of rack used, 500 μL of absolute EtOH is added to each tube using a pipette aid and a 5 mL pipette. Each tube is mixed by inversion until DNA precipitates. The DNA is then washed with 250 μL 70% EtOH. The DNA is then transferred into a tube containing 200 μL of TE buffer. To dissolve the DNA, tubes are shaken overnight at 50° C.

### Example 8

### Screening of Resulting Clones for Successful mDCAL Gene Deletion

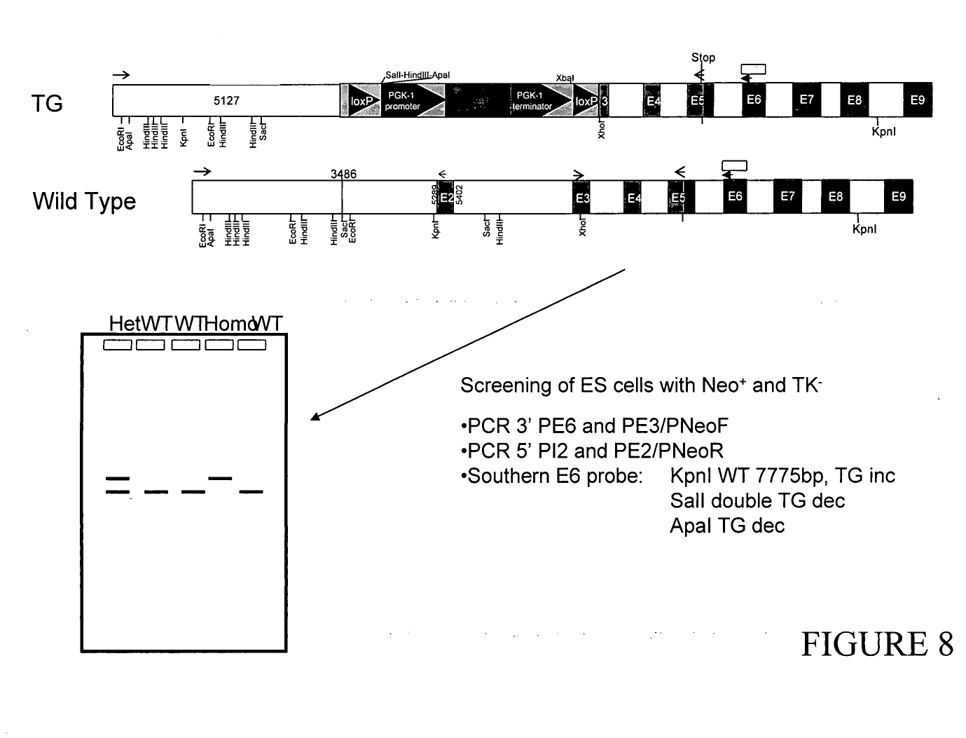

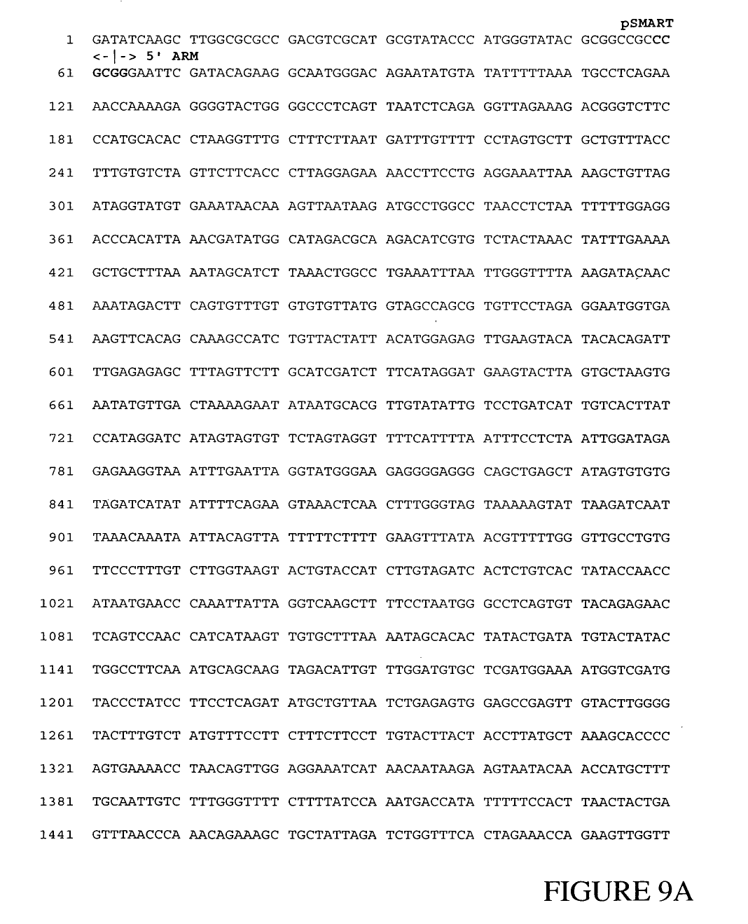

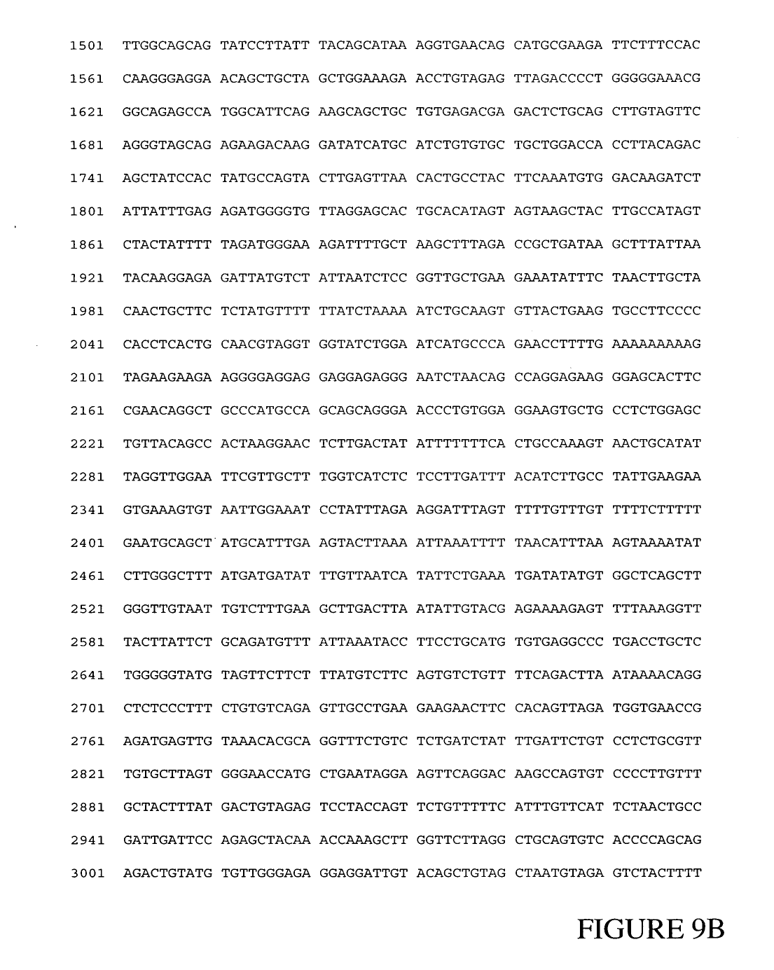

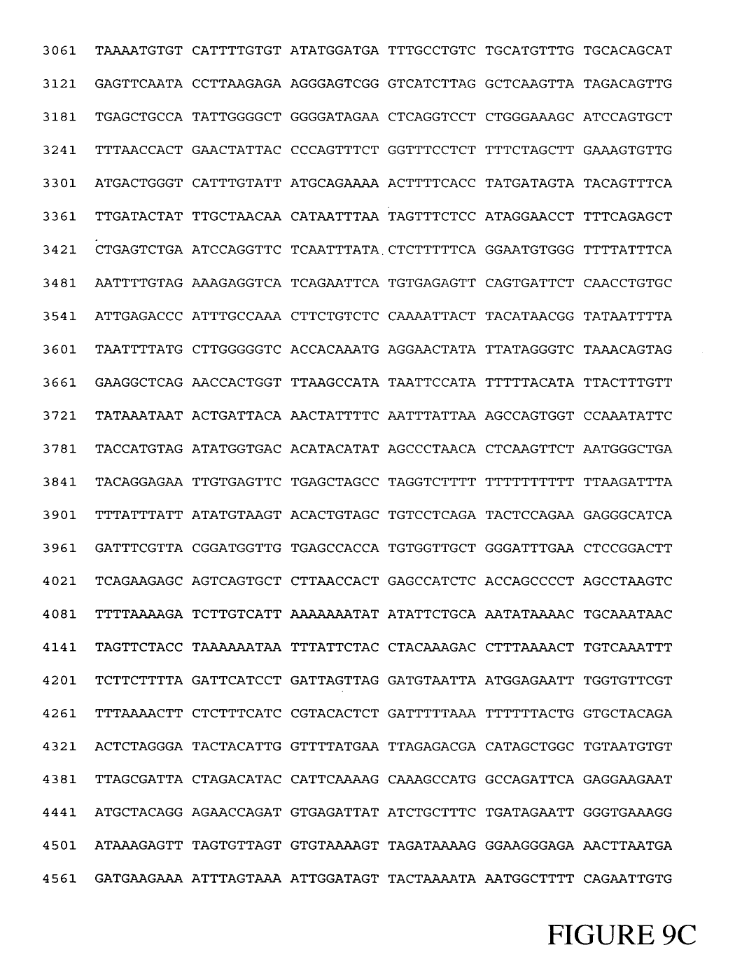

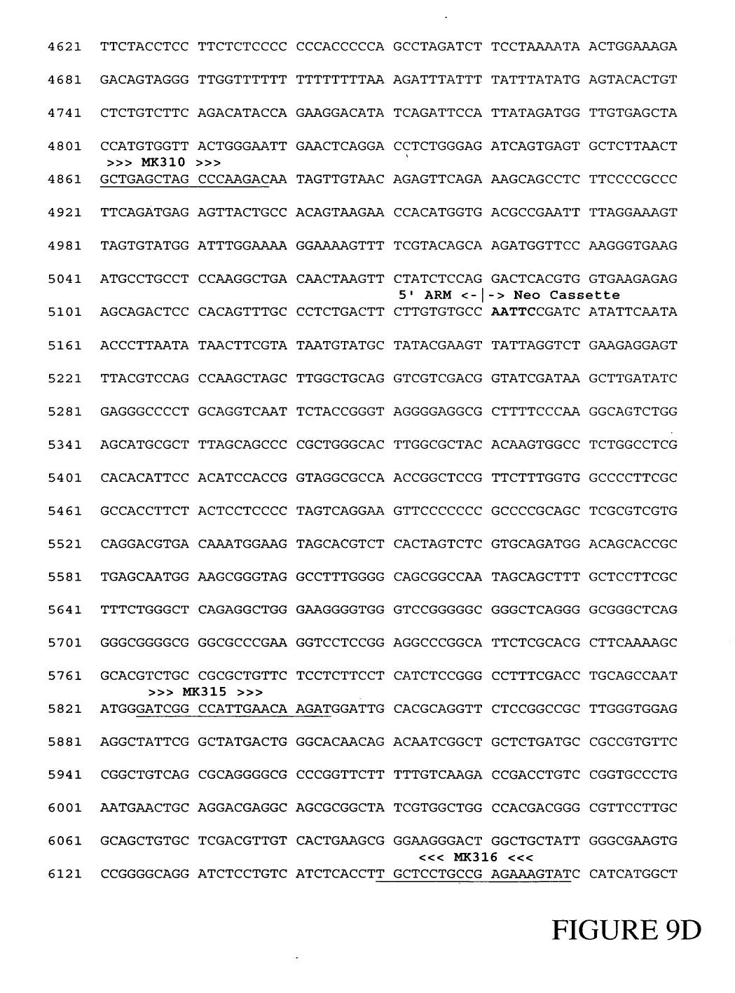

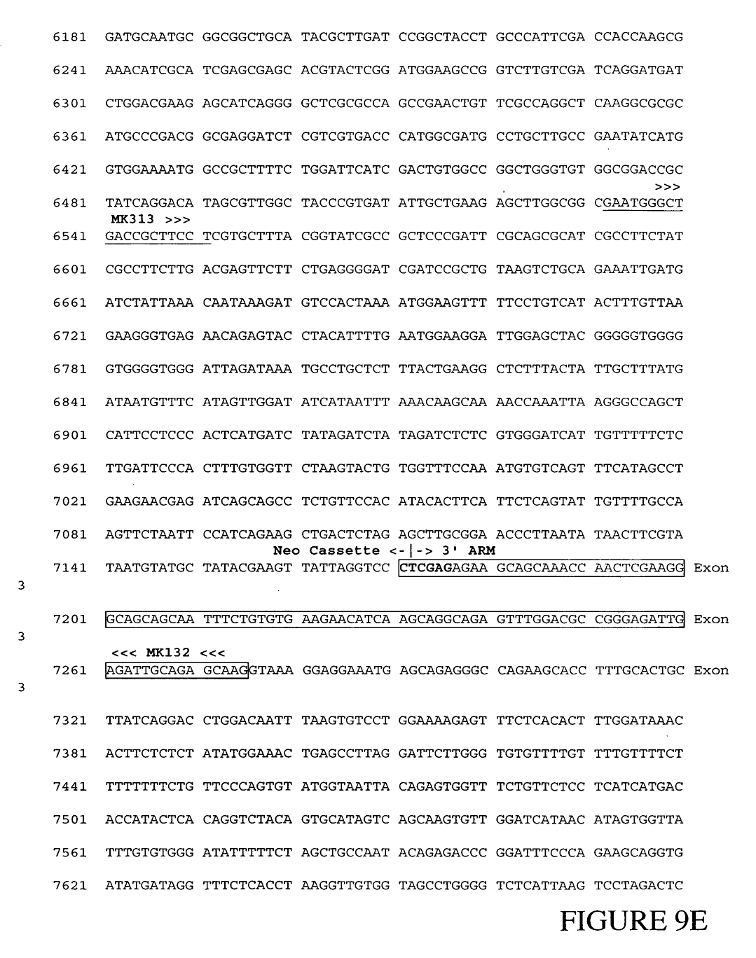

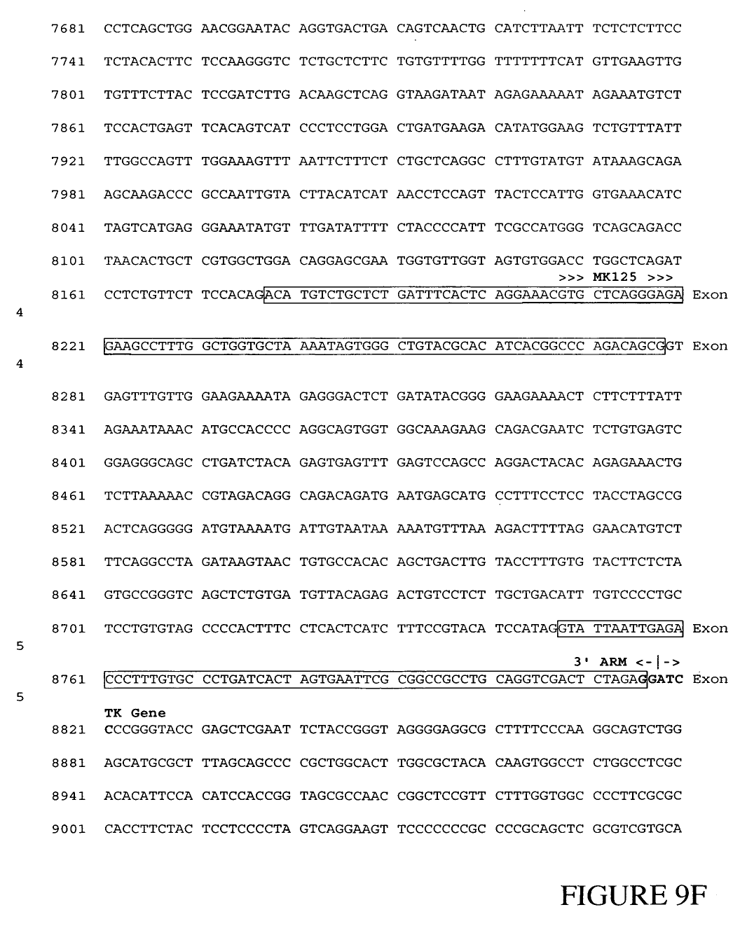

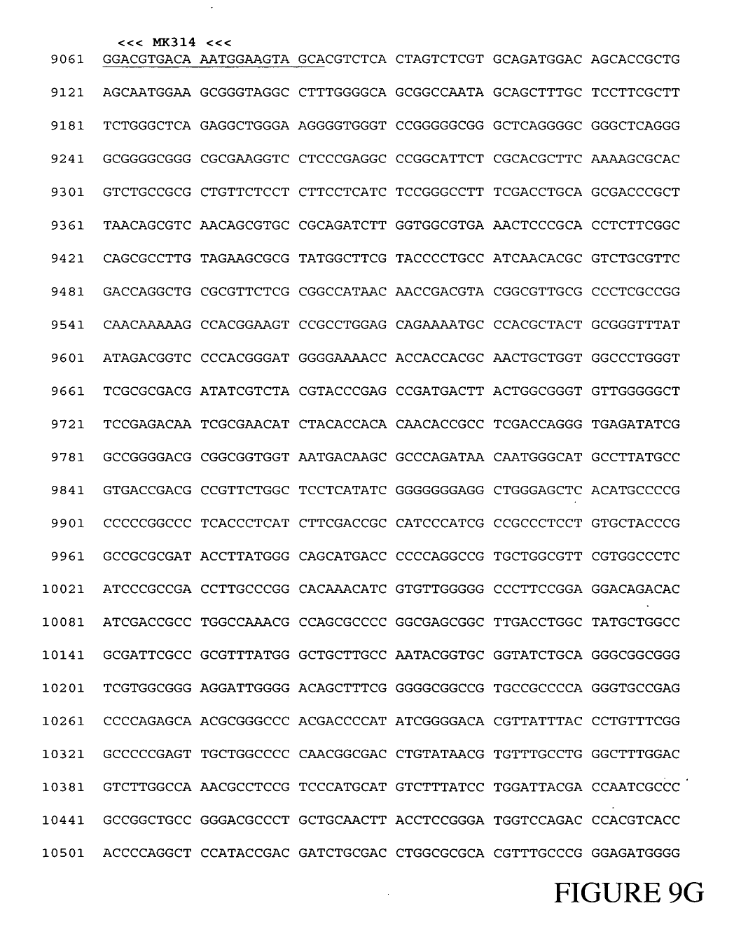

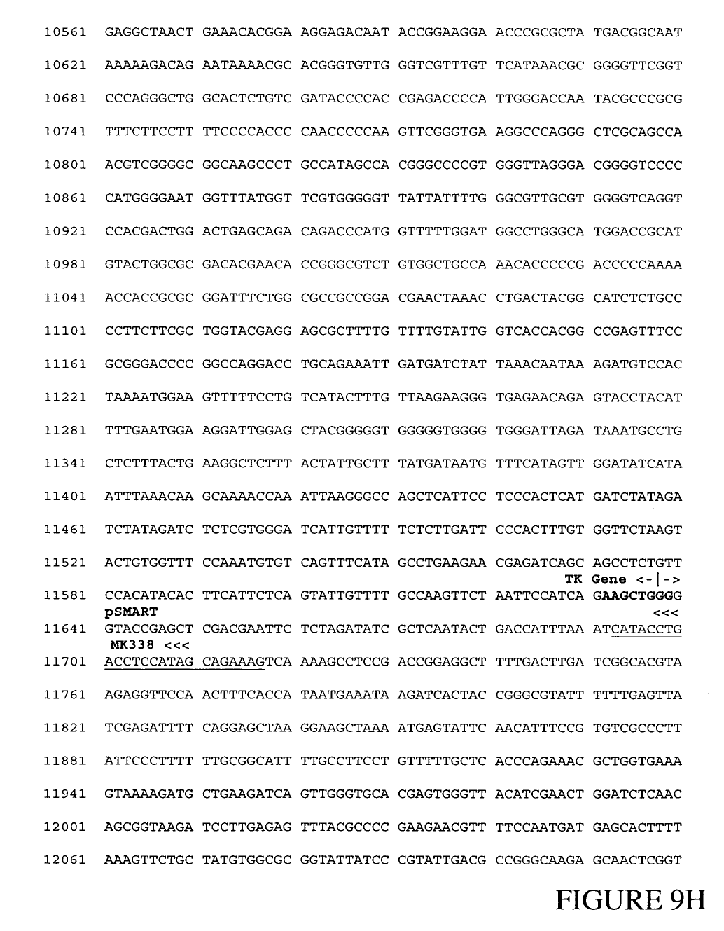

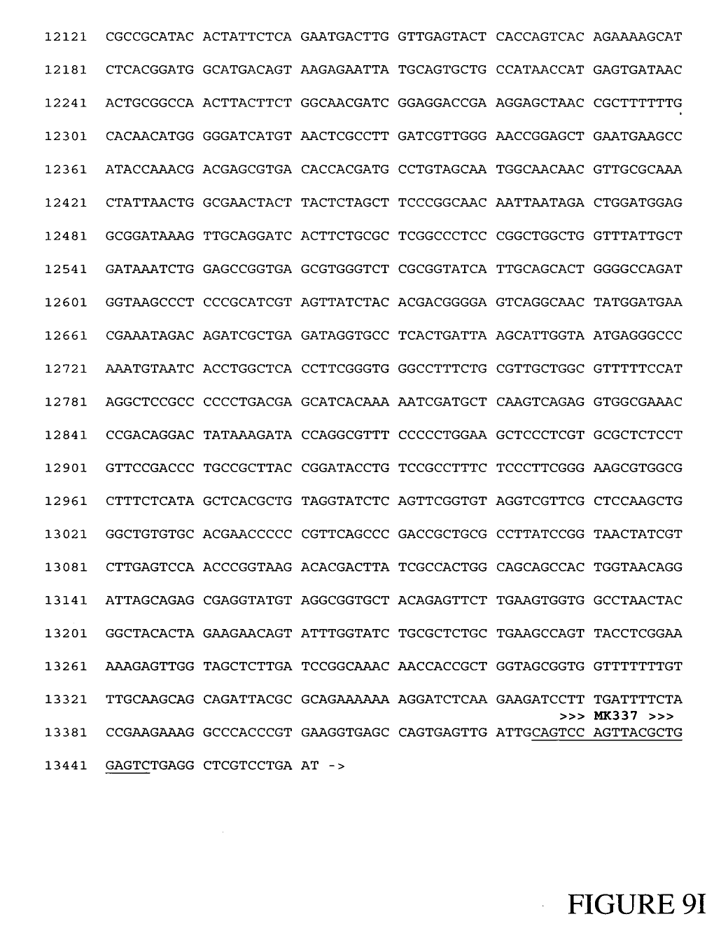

Resulting G418 resistant clones are digested with relevant diagnostic restriction enzymes and the digested DNA is then subjected to Southern hybridization as outlined in Sambrook et al. (supra). The probes utilized for this hybridization are a 3′ TG arm specific probe and a 5′ TG arm specific probe which have a differential size after digestion if the transgene has successfully inserted. (see FIG. 8) An additional Neo specific probe may also be used as confirmation. These results can be further confirmed with PCR screening using diagnostic primers across the proposed transgene insertion site.

Problems with ES cell viability/morphology due to effects on proximally located genes may require repeating the experiment again and using co-transfection of the transgene with a Cre-lox plasmid to remove the active Neo promoter. The resulting transfected cells will be cloned by limiting dilution and screened in the manner outlined above to determine successful TG cell lines.

### Example 9

### Harvesting of Cells for Blastocyst Injection

Cell cultures in logarithmic growth phase are employed. Two small (25-cm²) flasks are required, one is used for harvesting cells at T=0 and the other approx. 2 hours later, if there are sufficient blastocysts. Medium is removed from the flasks and cells are washed successively in 5 mL PBS and in 5 mL PBS/EGTA. PBS is then removed at 37° C. until cell boundaries can be seen. One mL of 0.25% trypsin/EDTA (1.6 mL of 2.5% Trypsin in 20 mL 0.05% Trypsin/EGTA) is then added, and the plates rocked back and forth for 1 min (by this time all the ES Cell colonies and feeders should be detached). Using a 1 mL pipette cells are dispersed until single cells can be seen (no longer than 1 min). The trypsin/EDTA is neutralized with 1 mL medium, cells are centrifuged for 3 min at 1000 rpm and resuspended at a concentration of 10⁶/mL in DMEM with Hepes. Fifty microlitres of cells are added to 1 mL DMEM with Hepes (see Media for embryo manipulation and culture) with 3000U DNase I.

### Example 10

### Blastocyst Transfer

Blastocyst transfer is usually performed 24 hours after aggregation when the morulae have become expanded blastocysts and on the same day as injection. A little time is given between injection and transfer to allow blastocysts to re-expand.

### Example 11

### Recipient

Two strains of mice are used: RB Swiss and (CBA*C57BL6/J)f1. RB Swiss are quiet and make excellent mothers but they become overweight quickly and do exhibit bad planes of anesthesia. CBA*C57 mice are hardy and display hybrid vigor. They do not carry excess weight and go under anesthetic well. This strain can be very nervous when housed separately which could be dangerous to their young. They are most suitable if a young RB Swiss is placed into the cage as a companion which can be removed as soon as the pups are seven days old. By this age destruction of the litter is very unlikely. If the CBA mother is to be housed alone she must not be disturbed for ten days.

Prior to surgery, sterilize the following:
3 pairs of curved forceps
2 pairs of iris scissors
1 pair suture clamps
1 serafin clip
sterile suture with needle attached (small—for mouse surgery)
Michelle clips (small size)
Michelle clip applicators
1 mouth pipette and flame-polished transfer pipette

An anaesthetic such as Rompun/Ketavet is employed to anaesthetize the animals. To make up 10 mL of anesthetic:
0.5 mL 2% Rompun (20 mg/mL Xylazine)
0.5 mL 100 mg/mL Ketavet 100—Delta Veterinary Lab, 8 Rosemead Rd Hornsby NSW 2077
Makeup to 10 mL with PBS

The dosage is 0.02 mL/g body weight Store wrapped in tin foil at 4° C. Shake well before use.

### Example 12

### Transfer

A mouse that is 2.5 days pseudo pregnant and weighs 25-30 g is selected for blastocyst transfer and anesthetized with Rompun/Ketavet, administered intraperitoneally. The lower back of the mouse is shaved and the shaven area is swabbed with hibitane or 70% ethanol. A small cut (about 1 cm long) is made along the dorsal midline of the lower back using a pair of scissors. The skin is separated from the body wall using forceps. If the cut has been made in the right place, the ovarian fat pad is easily visible. A serafin clip is attached to the fat pad, and the ovary, oviduct and part of the uterus are gently eased out through the incision in the body wall. When the tip of the uterus is visible, the serafin clip is rested across the mouse's back to hold the uterus in place.

A minute amount of DMEM with Hepes is loaded into the tip of the transfer pipette followed by a small bubble of air. This procedure is repeated before taking up a series of blastocysts in the smallest possible volume of medium. Once the pipette is loaded and the uterus positioned, the top of the uterine horn is gently grasped with a pair of forceps and a 26 gauge hypodermic needle gently inserted through the uterine wall (close to the oviduct) and into the lumen. The needle is removed carefully and the loaded transfer pipette gently inserted about 3 mm into the uterine lumen. The blastocysts are then gently delivered into the uterus, using the air bubbles in the transfer pipette to monitor the transfer. With the transfer complete, the serafin clip is removed and the uterus gently eased back into the body. This procedure is then repeated on the other uterine horn. The skin is closed with Michelle clips—two per incision is usually sufficient.

Once surgery is complete, the mouse is placed in a box of clean autoclaved sawdust. If pregnancy results, the pups are born sixteen days after transfer.

If this knockout mouse is determined to be embryonic lethal, this process will be repeated with construct B which contains the entire mDCAL gene with a portion of Exon 3 flanked by loxP sites. The mDCAL gene can then be inactivated in a cell specific manner/developmental time frame/inducible manner, by crossing the TG with Lox expressing transgenic mice.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### Bibliography

Ault-Riche, D. B., Yuan, C. S., and Borchardt, R. T.: A single mutation at lysine 426 of human placental S-adenosylhomocysteine hydrolase inactivates the enzyme. J. Biol. Chem. 269: 31472-8, 1994
Blom, N., Gammeltoft, S., and Brunak, S.: Sequence- and Structure-Based Prediction of Eukaryotic Protein Phosphorylation Sites. J. Mol. Biol. 294: 1351-1362, 1999
Burkly, L., Hession, C., Ogata, L., Reilly, C., Marconi, L. A., Olson, D., Tizard, R., Cate, R., and Lo, D.: Expression of relB is required for the development of thymic medulla and dendritic cells. Nature 373: 531-6, 1995
Chiang, P. K., Gordon, R. K., Tal, J., Zeng, G. C., Doctor, B. P., Pardhasaradhi, K., and McCann, P. P.: S-Adenosylmethionine and methylation. FASEB J. 10: 471-80, 1996
Clark, G. J., Gunningham, S., Troy, A., Vuckovic, S., and Hart, D. N.: Expression of the RelB transcription factor correlates with the activation of human dendritic cells. Immunol. 98: 189-96, 1999
Coulter-Karis, D. E. and Hershfield, M. S.: Sequence of full length cDNA for human S-adenosylhomocysteine hydrolase. Ann. Hum. Genet. 53: 169-75, 1989
Cumberbatch, M. and Kimber, I.: Dermal tumour necrosis factor-alpha induces dendritic cell migration to draining lymph nodes, and possibly provides one stimulus for Langerhans' cell migration. Immunol. 75: 257-63, 1992
de Saint-Vis, B., Vincent, J., Vandenabeele, S., Vanbervliet, B., Pin, J. J., Ait-Yahia, S., Patel, S., Mattei, M. G., Banchereau, J., Zurawski, S., Davoust, J., Caux, C., and Lebecque, S.: A novel lysosome-associated membrane glycoprotein, DC-LAMP, induced upon DC maturation, is transiently expressed in MHC class II compartment. Immunity 9: 325-36, 1998
DeLorenzi, M., Ali, N., Saari, G., Henry, C., Wilcox, M., and Bienz, M.: Evidence that the Abdominal-B r element function is conferred by a trans-regulatory homeoprotein. EMBO J. 7: 3223-31, 1988
Egner, W., McKenzie, J. L., Smith, S. M., Beard, M. E., and Hart, D. N.: Identification of potent mixed leukocyte reaction-stimulatory cells in human bone marrow. Putative differentiation stage of human blood dendritic cells. J. Immunol. 150: 3043-53, 1993
Fayette, J., Dubois, B., Vandenabeele, S., Bridon, J. M., Vanbervliet, B., Durand, I., Banchereau, J., Caux, C., and Briere, F.: Human dendritic cells skew isotype switching of CD40-activated naive B cells towards IgA1 and IgA2. J. Exp. Med. 185: 1909-18, 1997
Fearnley, D. B., McLellan, A. D., Mannering, S. I., Hock, B. D., and Hart, D. N.: Isolation of human blood dendritic cells using the CMRF-44 monoclonal antibody: implications for studies on antigen-presenting cell function and immunotherapy. Blood 89: 3708-16, 1997
Hart, D. N.: Dendritic cells: unique leukocyte populations which control the primary immune response. Blood 90: 3245-87, 1997
Hart, D. N. and McKenzie, J. L.: Isolation and characterization of human tonsil dendritic cells. J. Exp. Med. 168. 157-70, 1988
Hock, B. D., Fearnley, D. B., Boyce, A., McLellan, A. D., Sorg, R. V., Summers, K. L., and Hart, D. N.: Human dendritic cells express a 95 kDa activation/differentiation antigen defined by CMRF-56. Tissue Antigens 53: 320-34, 1999
Hock, B. D., Starling, G. C., Daniel, P. B., and Hart, D. N.: Characterization of CMRF-44, a novel monoclonal antibody to an activation antigen expressed by the allostimulatory cells within peripheral blood, including dendritic cells. Immunol. 83: 573-81, 1994
Ingulli, E., Mondino, A., Khoruts, A., and Jenkins, M. K.: In vivo detection of dendritic cell antigen presentation to CD4(+) T cells. J. Exp. Med. 185: 2133-41, 1997
Kato, M., Neil, T. K., Fearnley, D. B., McLellan, A. D., Vuckovic, S., and Hart, D. N.:

Expression of multilectin receptors and comparative FITC-dextran uptake by human dendritic cells. Int. Immunol. 12: 1511-1519, 2000
Mannering, S. I., McKenzie, J. L., and Hart, D. N.: Optimisation of the conditions for generating human DC initiated antigen specific T lymphocyte lines in vitro. J. Immunol. Methods 219: 69-83, 1998
McKenzie, J. L., Egner, W., Calder, V. L., and Hart, D. N.: Hodgkin's disease cell lines: a model for interleukin-1-independent accessory cell function [published erratum appears in Immunology 1992 December ; 77(4):628]. Immunol. 77: 345-53, 1992
McLellan, A. D., Heiser, A., and Hart, D. N.: Induction of dendritic cell costimulator molecule expression is suppressed by T cells in the absence of antigen-specific signalling: role of cluster formation, CD40 and HLA-class II for dendritic cell activation. Immunol. 98: 171-80, 1999
McLellan, A. D., Heiser, A., Sorg, R. V., Fearnley, D. B., and Hart, D. N.: Dermal dendritic cells associated with T lymphocytes in normal human skin display an activated phenotype. J. Invest. Dermatol. 111: 841-9, 1998
McLellan, A. D., Sorg, R. V., Williams, L. A., and Hart, D. N.: Human dendritic cells activate T lymphocytes via a CD40: CD40 ligand-dependent pathway. Eur. J. Immunol. 26: 1204-10, 1996
Merta, A., Aksamit, R. R., Kasir, J., and Cantoni, G. L.: The gene and pseudogenes of rat S-adenosyl-L-homocysteine hydrolase. Eur. J. Biochem. 229: 575-82, 1995
Miller, M. W., Duhl, D. M., Winkes, B. M., Arredondo-Vega, F., Saxon, P. J., Wolff, G. L., Epstein, C. J., Hershfield, M. S., and Barsh, G. S.: The mouse lethal nonagouti (a(x)) mutation deletes the S-adenosylhomocysteine hydrolase (Ahcy) gene. EMBO J. 13: 1806-16, 1994
Nagase, T., Ishikawa, K., Suyama, M., Kikuno, R., Hirosawa, M., Miyajima, N., Tanaka, A., Kotani, H., Nomura, N., and Ohara, O.: Prediction of the coding sequences of unidentified human genes. XII. The complete sequences of 100 new cDNA clones from brain which code for large proteins in vitro. DNA. Res. 5: 355-64, 1998
Romani, N., Koide, S., Crowley, M., Witmer-Pack, M., Livingstone, A. M., Fathman, C. G., Inaba, K., and Steinman, R. M.: Presentation of exogenous protein antigens by dendritic cells to T cell clones. Intact protein is presented best by immature, epidermal Langerhans cells. J. Exp. Med. 169: 1169-78, 1989
Sozzani, S., Sallusto, F., Luini, W., Zhou, D., Piemonti, L., Allavena, P., Van Damme, J., Valitutti, S., Lanzavecchia, A., and Mantovani, A.: Migration of dendritic cells in response to formyl peptides, C5a, and a distinct set of chemokines. J. Immunol. 155: 3292-5, 1995
Steinman, R. M.: The dendritic cell system and its role in immunogenicity. Annu. Rev. Immunol. 9: 271-96, 1991
Summers, K. L., Hock, B. D., McKenzie, J. L., and Hart, D. N.: Phenotypic characterization of five dendritic cell subsets in human tonsils. Am J Pathol 159: 285-95., 2001
Szabolcs, P., Avigan, D., Gezelter, S., Ciocon, D. H., Moore, M. A., Steinman, R. M., and Young, J. W.: Dendritic cells and macrophages can mature independently from a human bone marrow-derived, post-colony-forming unit intermediate. Blood 87: 4520-30, 1996
Turner, M. A., Yuan, C. S., Borchardt, R. T., Hershfield, M. S., Smith, G. D., and Howell, P. L.: Structure determination of selenomethionyl S-adenosylhomocysteine hydrolase using data at a single wavelength. Nat. Struct. Biol. 5: 369-76, 1998
Ueland, P. M.: Pharmacological and biochemical aspects of S-adenosylhomocysteine and S-adenosylhomocysteine hydrolase. Pharmacol. Rev. 34: 223-53, 1982
Vuckovic, S., Fearnley, D. B., Mannering, S. I., Dekker, J., Whyte, L. F., and Hart, D. N.: Generation of CMRF-44+ monocyte-derived dendritic cells: insights into phenotype and function. Exp. Hematol. 26: 1255-64, 1998
Welsh, J., Chada, K., Dalal, S. S., Cheng, R., Ralph, D., and McClelland, M.: Arbitrarily primed PCR fingerprinting of RNA. Nucleic Acids Res. 20: 4965-70, 1992
Yuan, C. S., Ault-Riche, D. B., and Borchardt, R. T.: Chemical modification and site-directed mutagenesis of cysteine residues in human placental S-adenosylhomocysteine hydrolase. J. Biol. Chem. 271: 28009-16, 1996
Zhou, L. J. and Tedder, T. F.: Human blood dendritic cells selectively express CD83, a member of the immunoglobulin superfamily. J. Immunol. 154: 3821-35, 1995

1. An isolated polynucleotide comprising a nucleotide sequence which corresponds or is complementary to at least a portion of the sequence set forth in SEQ ID NO: 1 or 5, wherein the portion is at least 18 nucleotides in length and comprises at least a fragment of an intron.

2. The polynucleotide of claim 1, wherein the nucleotide sequence has at least 60% sequence identity to the sequence set forth in SEQ ID NO: 1 or 3.

3. The polynucleotide of claim 1, wherein the nucleotide sequence is capable of hybridizing to at least a portion of the sequence set forth in SEQ ID NO: 1 or 5 under at least medium stringency conditions.

4. The polynucleotide of claim 3, wherein the nucleotide sequence further comprises at least one exon or a portion thereof.

5. A vector comprising the polynucleotide of claim 1.

6. The vector of claim 5, which is a DNA targeting vector.

7. A host cell containing the vector of claim 6.

8. A non-human genetically modified animal having altered DCAL function.

9. The genetically modified animal of claim 8, which has a partial or complete loss of function in one or both alleles of the endogenous DCAL gene.

10. The genetically modified animal of claim 8, which has an inducible or conditional partial or complete loss of function in one or both alleles of the endogenous DCAL gene.

11. The genetically modified animal of claim 10, which has a conditional DCAL knockout.

12. The genetically modified animal of claim 8, which comprises a disruption or which has an inducible or conditional disruption in at least one allele of the endogenous DCAL gene.

13. The genetically modified animal of claim 12, wherein the disruption or the inducible or conditional disruption has been introduced into its genome by homologous recombination with a DNA targeting construct in an embryonic stem cell such that the targeting construct is stably integrated in the genome of the animal, wherein the disruption or the inducible or conditional disruption of the DCAL gene results in an inability of the animal to produce a functional DCAL or detectable levels of DCAL.

14. A method for disrupting a DCAL gene in a cell, comprising:
providing a first polynucleotide having a sequence comprising: i) at least a portion of a DCAL gene; and ii) a second polynucleotide capable of disrupting the DCAL gene; and
introducing the first polynucleotide into a non-human cell under conditions such that the first polynucleotide is homologously recombined into at least one of the naturally occurring alleles of the DCAL gene in the genome of the cell to produce a cell containing at least one disrupted DCAL allele or at least one DCAL gene that is inducibly or conditionally disrupted.

15. The method of claim 14, which is a method for producing a non-human genetically modified animal containing at least one disrupted DCAL allele or at least one DCAL gene that is inducibly or conditionally disrupted.

16. The method of claim 15, wherein the non-human genetically modified animal containing the homologously recombined polynucleotide expresses or is capable of inducibly or conditionally expressing reduced or undetectable levels of DCAL.

17. The method of claim 15, wherein the non-human genetically modified animal lacks the ability to produce functional DCAL.

18. The method of claim 15, wherein the cell contains at least one DCAL gene that comprises target sites that are recognized by a recombinase enzyme and that are located within or adjacent to the DCAL gene, whereby excision of a nucleotide sequence between the target sites by the recombinase enzyme results in a reduction in the level or functional activity of a DCAL gene expression product.

19. The method of claim 15, wherein the DCAL gene is conditionally expressed in hematopoietic cells.

20. The method of claim 15, wherein the DCAL gene is conditionally expressed in dendritic cells.

21. A process for screening a candidate agent for the ability to specifically modulate DCAL function, comprising:
a. administering a candidate agent to the genetically modified non-human animal of claim 9 and to a corresponding wild-type non-human animal; and
b. comparing the immune response of the genetically modified animal to the immune response of the wild-type animal,
wherein a substantial absence in modulation of immune response in the genetically modified animal and a substantial modulation of immune response in the wild-type animal indicates that the candidate agent specifically modulates DCAL function.

22. A process for screening a candidate agent for the ability to specifically modulate DCAL function, comprising:
a. exposing first DC from the genetically modified non-human animal of claim 9 and second DC from a corresponding wild-type non-human animal to a candidate agent; and
b. comparing the individual immune responses of the first DC and of the second DC,
wherein a substantial absence of modulation in the immune response of the first DC and a substantial modulation in the immune response of the second DC indicates that the candidate agent specifically modulates DCAL function.

23. A method for modulating an immune response, the method comprising administering to a patient in need of such treatment a modulatory agent for a time and under conditions sufficient to modulate the immune response, wherein the modulatory agent is identifiable by a process comprising:
a. administering a candidate agent to the genetically modified non-human animal of claim 9 and to a corresponding wild-type non-human animal; and
b. comparing the immune response of the genetically modified animal to the immune response of the wild-type animal;
wherein a substantial absence in modulation of immune response in the genetically modified animal and a substantial modulation of immune response in the wild-type animal indicates that the candidate agent specifically modulates DCAL function.

24. The method of claim 23, wherein the agent reduces or suppresses the immune response.

25. A method for modulating an immune response, the method comprising administering to a patient in need of such treatment a modulatory agent for a time and under conditions sufficient to modulate the immune response, wherein the modulatory agent is identifiable by a process comprising:
a. exposing first DC from the genetically modified non-human animal of claim 10 and second DC from a corresponding wild-type non-human animal to a candidate agent; and
b. comparing the individual immune responses of the first DC and of the second DC,
wherein a substantial absence in modulation in the immune response of the first DC and a substantial modulation in the immune response of the second DC indicates that the candidate agent specifically modulates DCAL function.

26. The method of claim 25, wherein the agent reduces or suppresses the immune response.

27. A method of at least one for treatment and prophylaxis of a condition associated with expression or activation of DCAL, the method comprising administering to a patient in need of such treatment a therapeutically effective amount of an agent for a time and under conditions sufficient to reduce at least one of a level and functional activity of DCAL, wherein the agent is identifiable by a process comprising:
a. administering a candidate agent to the genetically modified non-human animal of claim 9 and to a corresponding wild-type non-human animal; and
b. comparing the immune response of the genetically modified animal to the immune response of the wild-type animal,
wherein a substantial absence in reduction of immune response in the genetically modified animal and a substantial reduction of immune response in the wild-type animal indicates that the candidate agent specifically reduces DCAL function.

28. The method of claim 27, wherein the condition is selected from the group consisting of an allergy, autoimmune disease and transplant rejection.

29. A method of at least one for treatment and prophylaxis of a condition associated with expression or activation of DCAL, the method comprising administering to a patient in need of such treatment a therapeutically effective amount of an agent for a time and under conditions sufficient to reduce at least one of a level and functional activity of DCAL, wherein the agent is identifiable by a process comprising:
a. exposing first DC from the genetically modified non-human animal of claim 10 and second DC from a corresponding wild-type non-human animal to a candidate agent; and
b. comparing the individual immune responses of the first DC and of the second DC,
wherein a substantial absence in reduction in the immune response of the first DC and a substantial reduction in the immune response of the second DC indicates that the candidate agent specifically reduces DCAL function.

30. The method of claim 29, wherein the condition is selected from the group consisting of an allergy, autoimmune disease and transplant rejection.
